(19) United States
(12) Patent Application Publication
(10) Pub. No.: US 2023/0193243 A1
(43) Pub. Date: Jun. 22, 2023

(54) COMPOSITIONS COMPRISING A CAS12I2 POLYPEPTIDE AND USES THEREOF

(71) Applicant: Arbor Biotechnologies, Inc., Cambridge, MA (US)

(72) Inventors: David A. SCOTT, San Francisco, CA (US); Kerri-Lynn SHEAHAN, Newton, MA (US); Tia Marie DITOMMASO, Waltham, MA (US); Quinton Norman WESSELLS, Cambridge, MA (US); Noah Michael JAKIMO, Cambridge, MA (US)

(21) Appl. No.: 18/000,218

(22) PCT Filed: May 28, 2021
(86) PCT No.: PCT/US21/34950

§ 371 Date: Nov. 29, 2022

Related U.S. Application Data
(60) Provisional application No. 63/032,315, filed on May 29, 2020.
(60) Provisional application No. 63/107,172, filed on Oct. 29, 2020.

(51) Int. Cl. C12N 15/10; C12N 15/11

(52) U.S. Cl. CPC C12N 15/102; C12N 15/111; C12N 2310/20

(57) ABSTRACT

The present invention relates to compositions comprising a Cas12i2 polypeptide and an RNA guide, processes for characterizing the compositions, cells comprising the compositions, and methods of using the compositions.

### SEQUENCE LISTING

The instant application contains a Sequence Listing which has been submitted electronically in ASCII format and is hereby incorporated by reference in its entirety. Said ASCII copy, created on May 28, 2021, is named 51451-010WO3_Sequence_Listing_5_25_21_ST25, and is 91,751 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art. Although this invention disclosed herein is not limited to specific advantages or functionalities, the invention provides a composition comprising (a) an RNA guide or a nucleic acid encoding the RNA guide, wherein the RNA guide comprises a direct repeat sequence and a spacer sequence; and (b) a Cas12i2 polypeptide or a nucleic acid encoding the Cas12i2 polypeptide, wherein the RNA guide forms a complex with the Cas12i2 polypeptide, and wherein the spacer sequence binds to a target sequence adjacent to a protospacer adjacent motif (PAM) sequence comprising 4 nucleotides.

In another aspect of the composition, the PAM sequence comprises the sequence 5′-NTTN-3′, wherein N is any nucleotide.

In another aspect of the composition, the PAM sequence comprises the sequence 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein N is any nucleotide, Y is C or T, B is any nucleotide except for A, D is any nucleotide except for C, and R is A or G.

In another aspect of the composition, the PAM sequence comprises the sequence 5′-CTTT-3′, 5′-CTTC-3′, 5′-GTTT-3′, 5′-GTTC-3′, 5′-TTTC-3′, 5′-GTTA-3′, or 5′-GTTG-3′.

In another aspect of the composition, the spacer sequence comprises between 10 and 50 nucleotides in length.

In another aspect of the composition, the spacer sequence comprises between 15 and 35 nucleotides in length.

In another aspect of the composition, a. nucleotide 1 through nucleotide 5 of the spacer sequence comprise have 100% complementarity to the target nucleic acid; b. nucleotide 6 through nucleotide 10 of the spacer sequence comprise at least 80% complementarity to the target nucleic acid; c. nucleotide 11 through nucleotide 15 of the spacer sequence comprise at least 80% complementarity to the target nucleic acid; d. nucleotide 16 through nucleotide 20 of the spacer sequence comprise at least 60% complementarity to the target nucleic acid; e. nucleotide 1 through nucleotide 10 of the spacer sequence comprise at least 80% complementarity to the target nucleic acid; f. nucleotide 1 through nucleotide 15 of the spacer sequence comprise at least 80% complementarity to the target nucleic acid; g. nucleotide 1 through nucleotide 20 of the spacer sequence comprise at least 80% complementarity to the target nucleic acid; h. nucleotide 5 through nucleotide 15 of the spacer sequence comprise at least 80% complementarity to the target nucleic acid; i. nucleotide 5 through nucleotide 20 of the spacer sequence comprise at least 80% complementarity to the target nucleic acid; and/or j. nucleotide 10 through nucleotide 20 of the spacer sequence comprise at least 60% complementarity to the target nucleic acid.

In another aspect of the composition, the direct repeat sequence comprises at least 90% identity o SEQ ID NO: 18 or SEQ ID NO: 19

In another aspect of the composition, the direct repeat sequence comprises at least 95% identity to SEQ ID NO: 18 or SEQ ID NO: 19.

In another aspect of the composition, the direct repeat sequence comprises SEQ ID NO: 18 or SEQ ID NO: 19.

In another aspect of the composition, the direct repeat sequence comprises: a. nucleotide 1 through nucleotide 36 of a sequence that is at least 90% identical to a sequence of any one of SEQ ID NOs: 8-15; b. nucleotide 2 through nucleotide 36 of a sequence that is at least 90% identical to a sequence of any one of SEQ ID NOs: 8-15; c. nucleotide 3 through nucleotide 36 of a sequence that is at least 90% identical to a sequence of any one of SEQ ID NOs: 8-15; d. nucleotide 4 through nucleotide 36 of a sequence that is at least 90% identical to a sequence of any one of SEQ ID NOs: 8-15; e. nucleotide 5 through nucleotide 36 of a sequence that is at least 90% identical to a sequence of any one of SEQ ID NOs: 8-15; f. nucleotide 6 through nucleotide 36 of a sequence that is at least 90% identical to a sequence of any one of SEQ ID NOs: 8-15; g. nucleotide 7 through nucleotide 36 of a sequence that is at least 90% identical to a sequence of any one of SEQ ID NOs: 8-15; h. nucleotide 8 through nucleotide 36 of a sequence that is at least 90% identical to a sequence of any one of SEQ ID NOs: 8-15; i. nucleotide 9 through nucleotide 36 of a sequence that is at least 90% identical to a sequence of any one of SEQ ID NOs: 8-15; j. nucleotide 10 through nucleotide 36 of a sequence that is at least 90% identical to a sequence of any one of SEQ ID NOs: 8-15; k. nucleotide 11 through nucleotide 36 of a sequence that is at least 90% identical to a sequence of any one of SEQ ID NOs: 8-15; l. nucleotide 12 through nucleotide 36 of a sequence that is at least 90% identical to a sequence of any one of SEQ ID NOs: 8-15; m. nucleotide 13 through nucleotide 36 of a sequence that is at least 90% identical to a sequence of any one of SEQ ID NOs: 8-15; n. nucleotide 14 through nucleotide 36 of a sequence that is at least 90% identical to a sequence of any one of SEQ ID NOs: 8-15; o. nucleotide 1 through nucleotide 34 of a sequence that is at least 90% identical to a sequence of SEQ ID NO: 16; p. nucleotide 2 through nucleotide 34 of a sequence that is at least 90% identical to a sequence of SEQ ID NO: 16; q. nucleotide 3 through nucleotide 34 of a sequence that is at least 90% identical to a sequence of SEQ ID NO: 16; r. nucleotide 4 through nucleotide 34 of a sequence that is at least 90% identical to a sequence of SEQ ID NO: 16; s. nucleotide 5 through nucleotide 34 of a sequence that is at least 90% identical to a sequence of SEQ ID NO: 16; t. nucleotide 6 through nucleotide 34 of a sequence that is at least 90% identical to a sequence of SEQ ID NO: 16; u. nucleotide 7 through nucleotide 34 of a sequence that is at least 90% identical to a sequence of SEQ ID NO: 16; v. nucleotide 8 through nucleotide 34 of a sequence that is at least 90% identical to a sequence of SEQ ID NO: 16; w. nucleotide 9 through nucleotide 34 of a sequence that is at least 90% identical to a sequence of SEQ ID NO: 16; x. nucleotide 10 through nucleotide 34 of a sequence that is at least 90% identical to a sequence of SEQ ID NO: 16; y. nucleotide 11 through nucleotide 34 of a sequence that is at least 90% identical to a sequence of SEQ ID NO: 16; z. nucleotide 12 through nucleotide 34 of a sequence that is at least 90% identical to a sequence of SEQ ID NO: 16; or aa. a sequence that is at least 90% identical to a sequence of SEQ ID NO: 17 or a portion thereof.

In another aspect of the composition, the direct repeat sequence comprises: a. nucleotide 1 through nucleotide 36 of any one of SEQ ID NOs: 8-15; b. nucleotide 2 through nucleotide 36 of any one of SEQ ID NOs: 8-15; c. nucleotide 3 through nucleotide 36 of any one of SEQ ID NOs: 8-15; d. nucleotide 4 through nucleotide 36 of any one of SEQ ID NOs: 8-15; e. nucleotide 5 through nucleotide 36 of any one of SEQ ID NOs: 8-15; f. nucleotide 6 through nucleotide 36 of any one of SEQ ID NOs: 8-15; g. nucleotide 7 through nucleotide 36 of any one of SEQ ID NOs: 8-15; h. nucleotide 8 through nucleotide 36 of any one of SEQ ID NOs: 8-15; i. nucleotide 9 through nucleotide 36 of any one of SEQ ID NOs: 8-15; j. nucleotide 10 through nucleotide 36 of any one of SEQ ID NOs: 8-15; k. nucleotide 11 through nucleotide 36 of any one of SEQ ID NOs: 8-15; l. nucleotide 12 through nucleotide 36 of any one of SEQ ID NOs: 8-15; m. nucleotide 13 through nucleotide 36 of any one of SEQ ID NOs: 8-15; n. nucleotide 14 through nucleotide 36 of any one of SEQ ID NOs: 8-15; o. nucleotide 1 through nucleotide 34 of SEQ ID NO: 16; p. nucleotide 2 through nucleotide 34 of SEQ ID NO: 16; q. nucleotide 3 through nucleotide 34 of SEQ ID NO: 16; r. nucleotide 4 through nucleotide 34 of SEQ ID NO: 16; s. nucleotide 5 through nucleotide 34 of SEQ ID NO: 16; t. nucleotide 6 through nucleotide 34 of SEQ ID NO: 16; u. nucleotide 7 through nucleotide 34 of SEQ ID NO: 16; v. nucleotide 8 through nucleotide 34 of SEQ ID NO: 16; w. nucleotide 9 through nucleotide 34 of SEQ ID NO: 16; x. nucleotide 10 through nucleotide 34 of SEQ ID NO: 16; y. nucleotide 11 through nucleotide 34 of SEQ ID NO: 16; z. nucleotide 12 through nucleotide 34 of SEQ ID NO: 16; or aa. SEQ ID NO: 17 or a portion thereof.

In another aspect of the composition, the direct repeat sequence comprises: a. a first portion having at least 90% identity to nucleotides 1-13 of SEQ ID NO: 17 and a second portion having at least 90% identity to nucleotides 14-23; b. a first portion having at least 90% identity to nucleotides 1-14 of SEQ ID NO: 17 and a second portion having at least 90% identity to nucleotides 15-23; c. a first portion having at least 90% identity to nucleotides 1-15 of SEQ ID NO: 17 and a second portion having at least 90% identity to nucleotides 16-23; or d. a first portion having at least 90% identity to nucleotides 1-16 of SEQ ID NO: 17 and a second portion having at least 90% identity to nucleotides 17-23; and a heterologous sequence between the first portion and the second portion.

In another aspect of the composition, the direct repeat sequence comprises: a. a first portion comprising nucleotides 1-13 of SEQ ID NO: 17 and a second portion comprising nucleotides 14-23; b. a first portion comprising nucleotides 1-14 of SEQ ID NO: 17 and a second portion comprising nucleotides 15-23; c. a first portion comprising nucleotides 1-15 of SEQ ID NO: 17 and a second portion comprising nucleotides 16-23; or d. a first portion comprising nucleotides 1-16 of SEQ ID NO: 17 and a second portion comprising nucleotides 17-23; and a heterologous sequence between the first portion and the second portion.

In another aspect of the composition, the heterologous sequence comprises a DNA sequence, an RNA sequence, or a DNA/RNA hybrid sequence. In another aspect of the composition, the heterologous sequence is an aptamer.

In another aspect of the composition, the direct repeat sequence comprises a stem-loop structure proximal to a 3′ end of the direct repeat sequence, wherein the stem-loop structure comprises a first stem nucleotide strand, a second stem nucleotide strand, and a loop nucleotide strand between the first stem nucleotide strand and the second stem nucleotide strand.

In another aspect of the composition, the first stem nucleotide strand comprises 3 to 5 nucleotides, the second stem nucleotide strand comprises 3 to 5 nucleotides, and the loop nucleotide strand comprises 7 to 11 nucleotides.

In another aspect of the composition, at least 3 nucleotides of the first stem nucleotide strand are complementary to at least 3 nucleotides of the second nucleotide stem strand.

In another aspect of the composition, at least 4 nucleotides of the first stem nucleotide strand are complementary to at least 4 nucleotides of the second nucleotide stem strand.

In another aspect of the composition, the first stem nucleotide strand is substantially complementary to the second nucleotide stem strand.

In another aspect of the composition, the Cas12i2 polypeptide comprises an amino acid sequence with at least 90% identity to SEQ ID NO: 2.

In another aspect of the composition, the Cas12i2 polypeptide comprises an amino acid sequence with at least 95% identity to SEQ ID NO: 2.

In another aspect of the composition, the Cas12i2 polypeptide comprises an amino acid sequence set forth in any one of SEQ ID NOs: 3-7.

In another aspect of the composition, the Cas12i2 polypeptide comprises at least one of an epitope peptide, a nuclear localization signal, and a nuclear export signal.

In another aspect of the composition, the target sequence is present in a cell.

In another aspect of the composition, the composition is formulated for delivery to a cell.

In another aspect of the composition, the Cas12i2 polypeptide and the RNA guide are encoded in a vector, e.g., one or more expression vectors.

In another aspect of the composition, the composition demonstrates increased binding to the target sequence adjacent to the PAM sequence comprising 4 nucleotides, as compared to a sequence that is not adjacent to a PAM sequence of the disclosure.

In another aspect of the composition, the composition demonstrates increased binding affinity to the target sequence, as compared to a sequence that is not adjacent to a PAM sequence of the disclosure.

In another aspect of the composition, the composition demonstrates increased RNA-DNA interactions with the target sequence, as compared to a sequence that is not adjacent to a PAM sequence of the disclosure.

In another aspect of the composition, the composition demonstrates decreased dissociation from the target sequence, as compared to a sequence that is not adjacent to a PAM sequence of the disclosure.

In another aspect of the composition, the composition demonstrates increased enzymatic activity at the target sequence, as compared to a sequence that is not adjacent to a PAM sequence of the disclosure.

In another aspect of the composition, the composition demonstrates decreased binding or binding affinity to a non-target sequence, as compared to a composition that binds a sequence that is not adjacent to a PAM sequence of the disclosure.

In another aspect of the composition, the PAM sequence of the disclosure does not comprise the sequence 5′-NVVN-3′, wherein V is any nucleotide except for T.

The invention also provides a vector comprising a sequence encoding the Cas12i2 polypeptide and RNA guide of the composition as disclosed herein.

The invention also provides a cell comprising the composition or vector as disclosed herein.

The invention also provides a method of expressing the vector as disclosed herein.

The invention also provides a method of producing composition disclosed herein.

The invention also provides a method of delivering the composition disclosed herein.

The invention also provides a method of binding the composition disclosed herein with the target sequence.

The invention also provides a method of targeting a sequence adjacent to a PAM sequence comprising four nucleotides, the method comprising contacting the sequence with a complex of as disclosed herein.

In one aspect of the method of targeting, the PAM sequence comprises the sequence 5′-NTTN-3′, wherein N is any nucleotide.

The invention also provides a method of designing an RNA guide for targeting a target sequence, the method comprising identifying a PAM sequence comprising the sequence 5′-NTTN-3′ adjacent to the target sequence, wherein N is any nucleotide, and designing or preparing a DNA-targeting sequence to be substantially complementary to a sequence comprising the sequence of the target sequence.

In one aspect of the methods of targeting or designing, the PAM sequence comprises the sequence 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein N is any nucleotide, Y is C or T, B is any nucleotide except for A, D is any nucleotide except for C, and R is A or G.

In another aspect of the methods of targeting or designing, the PAM sequence comprises the sequence 5′-CTTT-3′, 5′-CTTC-3′, 5′-GTTT-3′, 5′-GTTC-3′, 5′-TTTC-3′, 5′-GTTA-3′, or 5′-GTTG-3′.

The invention also provides a kit or system comprising a complex, composition, cell, or a vector as disclosed herein.

### Definitions

The present invention will be described with respect to particular embodiments and with reference to certain Figures, but the invention is not limited thereto but only by the claims. Terms as set forth hereinafter are generally to be understood in their common sense unless indicated otherwise.

As used herein, the term “activity” refers to a biological activity. In some embodiments, the activity refers to effector activity. In some embodiments, activity includes enzymatic activity, e.g., catalytic ability of an effector. For example, activity can include nuclease activity. In some embodiments, activity includes binding activity, e.g., binding activity of an effector to an RNA guide and/or target nucleic acid.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences. In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, the term “adjacent to” is used to refer a protein residue that interacts with another protein residue. In some embodiments, the term “adjacent to” is used to refer a protein residue that interacts with a nucleotide or nucleic acid. In some embodiments, the term “adjacent to” is used to refer to a protein domain or motif that interacts with another protein domain or motif. In some embodiments, the term “adjacent to” is used to refer to a protein domain or motif that interacts with a nucleotide or nucleic acid sequence. As used herein, the term “adjacent to” is used to refer to the positioning of an indel (insertion/deletion) in a modified cell of the disclosure.

As used herein, the term “complex” refers to a grouping of two or more molecules. In some embodiments, the complex comprises a polypeptide and a nucleic acid molecule interacting with (e.g., binding to, coming into contact with, adhering to) one another. As used herein, the term “binary complex” refers to a grouping of two molecules (e.g., a polypeptide and a nucleic acid molecule). In some embodiments, a binary complex refers to a grouping of a polypeptide and an RNA guide. In some embodiments, a binary complex refers to a ribonucleoprotein (RNP). As used herein, the term “ternary complex” refers to a grouping of three molecules (e.g., a polypeptide and two nucleic acid molecules). In some embodiments, a “ternary complex” refers to a grouping of a polypeptide, an RNA molecule, and a DNA molecule. In some embodiments, a ternary complex refers to a grouping of a polypeptide, an RNA guide, and a target nucleic acid (e.g., a target DNA molecule). In some embodiments, a “ternary complex” refers to a grouping of a binary complex (e.g., a ribonucleoprotein) and a third molecule (e.g., a target nucleic acid).

As used herein, the term “protospacer adjacent motif” or “PAM” refers to a DNA sequence adjacent to a target sequence to which a binary complex comprising a polypeptide (e.g., an enzyme such as Cas12i2 or a variant thereof) and an RNA guide binds. In some embodiments, a PAM sequence is required for enzyme activity. In the case of a double-stranded target, the RNA guide binds to a first strand of the target, and a PAM sequence as described herein is present in the second, complementary strand. For example, in some embodiments, the RNA guide binds to the target strand (e.g., the spacer-complementary strand), and the PAM sequence as described herein is present in the non-target strand (i.e., the non-spacer-complementary strand).

As used herein, a “PAM sequence of the disclosure” is a PAM sequence comprising four nucleotides and, in some embodiments, a PAM sequence of the disclosure comprises the sequence 5′-NTTN-3′ wherein N is any nucleotide (e.g., A, G, T, or C). A PAM sequence of the disclosure can also consist of 2 or 3 nucleotides. In particular, a PAM sequence of the disclosure can comprise the sequence 5′-TTY-3′ or 5′-TTB-3′, wherein Y is C or T, and B is G, T, or C. In other embodiments, a PAM sequence of the disclosure consists of two nucleotides: 5′-NN-3′, wherein N is any nucleotide (e.g., A, T, C, or G), for example, 5′-TT-3′. A PAM sequence of the disclosure can optionally include one or more (e.g., 1, 2, 3, 4, 5, 6, 7, 8, 9, or 10) additional nucleotides 5′ of the sequence 5′-NTTN-3′, 5′-TTY-3′, or 5′TTB-3′. These one or more additional nucleotides can be N's and thus can each be any nucleotide (e.g., A, G, T, or C) or a subset thereof (e.g., R (G or A), Y (C or T), K (G or T), M (A or C), S (G or C), W (A or T), B (G, T, or C), D (G, A, or T), H (A, C, or T), or V (G, C, or A)). Examples of PAM sequences of the disclosure are set forth in Table 3, below.

As used herein, a “different PAM sequence” refers to a PAM sequence that is not a “PAM sequence of the disclosure” as defined herein. A “different PAM sequence” thus includes sequences comprising 5′-NXXN-3′ wherein X is not T.

As used herein, the term “sequence that is not adjacent to a PAM sequence of the disclosure” refers to a sequence lacking a PAM-adjacent sequence or a sequence that is adjacent to a different PAM sequence. The term is used in comparisons between a binary complex binding to a target sequence adjacent to a PAM sequence of the disclosure and a binary complex binding to a target sequence that is not adjacent to a PAM sequence of the disclosure. In these comparisons, the target sequence can be the same sequence. In the case of a double-stranded target, the RNA guide of the binary complex binds to a first strand of the target (i.e., the target strand or the spacer-complementary strand), and a PAM sequence as described herein or a different PAM sequence is present (or not) in the second, complementary strand (i.e., the non-target strand or the non-spacer-complementary strand).

As used herein, the terms “parent,” “parent polypeptide,” and “parent sequence” refer to an original polypeptide (e.g., starting polypeptide) to which an alteration is made to produce a variant Cas12i2 polypeptide of the present invention. In some embodiments, the parent is a polypeptide having an identical amino acid sequence of the variant with one or more variations at one or more specified positions. In exemplary embodiments, variations refer to amino acid changes within the polypeptide sequence. The parent may be a naturally occurring (wild-type) polypeptide. In a particular embodiment, the parent is a polypeptide with at least 60%, at least 61%, at least 62%, at least 63%, at least 64%, at least 65%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% identity to a polypeptide of SEQ ID NO: 2. In some embodiments, variations may include structural changes suck as linkages, fusions, or other changes that do not alter the original amino acid sequence of the parent. In some embodiments, the parent polypeptide sequence includes amino acid variations and structural changes.

As used herein, the terms “reference composition,” “reference molecule,” “reference sequence,” “reference,” and “reference complex” refer to a control, such as a negative control or a parent (e.g., a parent sequence, a parent protein, a wild-type protein, or a complex comprising a parent sequence). For example, a reference molecule refers to a Cas12i2 polypeptide to which a variant Cas12i2 polypeptide is compared. Likewise, a reference RNA guide refers to an RNA guide to which a modified RNA guide is compared. The variant or modified molecule may be compared to the reference molecule on the basis of sequence (e.g., the variant or modified molecule may have X % sequence identity or homology with the reference molecule), thermostability, or activity (e.g., the variant or modified molecule may have X % of the activity of the reference molecule).

As used herein, the terms “RNA guide” or “RNA guide sequence” refer to any RNA molecule that facilitates the targeting of a polypeptide described herein to a target nucleic acid. For example, an RNA guide can be a molecule that recognizes (e.g., binds to) a target nucleic acid. An RNA guide may be designed to be complementary to a specific nucleic acid sequence. An RNA guide comprises a DNA-targeting sequence and a direct repeat (DR) sequence. The terms CRISPR RNA (crRNA), pre-crRNA and mature crRNA are also used herein to refer to an RNA guide.

As used herein, the term “substantially complementary” refers to a polynucleotide (e.g., a spacer sequence of an RNA guide) that has a certain level of complementarity to a target sequence. In some embodiments, the level of complementarity is such that the polynucleotide can hybridize to the target sequence with sufficient affinity to permit an effector polypeptide (e.g., Cas12i2 or a variant thereof) that is complexed with the polynucleotide to act on (e.g., cleave) the target sequence.

As used herein, the terms “target sequence,” “target nucleic acid,” “target locus,” “target substrate,” and “on-target sequence” refer to a nucleic acid sequence to which an RNA guide specifically binds. In some embodiments, the DNA-targeting sequence of an RNA guide binds to a target sequence. Binding of a binary complex to a target sequence is referred to herein as “on-target binding.” In the case of a double-stranded target, the RNA guide binds to a first strand of the target (i.e., the target strand or the spacer-complementary strand), and a PAM sequence as described herein is present in the second, complementary strand (i.e., the non-target strand or the non-spacer-complementary strand). As used herein, the terms “non-target” and “off-target” refer to a nucleic acid sequence other than the sequence to which an RNA guide specifically binds or is intended to specifically bind. A non-target sequence is an unintended target of an RNA guide. Binding of a binary complex to a non-target sequence is referred to herein as “off-target binding.” In some embodiments, a non-target sequence is a non-target locus of a target sequence. In some embodiments, a non-target sequence is a locus on a nucleic acid other than the target sequence (e.g., a non-target sequence).

As used herein, the terms “variant Cas12i2 polypeptide” and “variant effector polypeptide” refer to a polypeptide comprising an alteration, e.g., a substitution, insertion, deletion and/or fusion, at one or more residue positions, compared to a parent polypeptide. As used herein, the terms “variant Cas12i2 polypeptide” and “variant effector polypeptide” refer to a polypeptide comprising an alteration as compared to the polypeptide of SEQ ID NO: 2.

### BRIEF DESCRIPTION OF THE DRAWINGS

FIG. 1A is a schematic showing the design of system for screening for Cas12i2 PAM sequences in E. coli. CRISPR array libraries were designed to include non-repetitive spacers uniformly sampled from both strands of pACYC184 or E. coli essential genes, flanked by two DRs, and expressed by a J23119 promoter.

FIG. 1B is a schematic showing the workflow of the in vivo E. coli screen. CRISPR array libraries were cloned into the effector plasmid. The effector plasmid and, when present, the non-coding plasmid, were transformed into E. coli followed by outgrowth for negative selection of CRISPR arrays conferring interference against DNA or RNA transcripts from pACYC184 or E. coli essential genes. Targeted sequencing of the effector plasmid was used to identify depleted CRISPR arrays.

FIG. 2A is a schematic showing the self-targeting design of a PAM library in a lentiviral vector. Each sequence flanking the target sequence) is SEQ ID NO: 75.

FIG. 2B is a schematic showing the process of library amplification, lentiviral packaging, and determination of indels.

FIG. 3 is an alignment of 5′ PAM motifs identified from the lentiviral screen. Enrichment of a base is indicated by larger font size, and the position of the target sequence is indicated.

FIG. 4A is a graph showing the percent indels for a variant Cas12i2 (SEQ ID NO: 3) using the CTTT PAM (circles) and the TTN PAM (triangles). Open circles/triangles represent AAVS1 targets, closed circles/triangles represent EMX1 targets, and half shaded circles/triangles represent VEGFA targets. The bars represented median indels across all targets tested

FIG. 4B is a graph showing the percent indels measured for wild-type Cas12i2 and a variant Cas12i2 (SEQ ID NO: 3) using the CTTT PAM (circles) and DTTR PAM (triangles). Open circles/triangles represent AAVS1 targets, closed circles/triangles represent EMX1 targets, and half shaded circles/triangles represent VEGFA targets. The bars represented median indels across all targets tested.

FIG. 5A shows percent indels measured for a variant Cas12i2 (SEQ ID NO: 4) at an AAVS1 target; each RNA guide was designed to have a single mismatch between the spacer sequence and the AAVS1 target sequence. FIG. 5B shows percent indels measured for a variant Cas12i2 (SEQ ID NO: 4) at an EMX1 target; each RNA guide was designed to have a single mismatch between the spacer sequence and the EMX1 target sequence. FIG. 5C shows percent indels measured for a variant Cas12i2 (SEQ ID NO: 4) at an AAVS1 target; each RNA guide was designed to have double, consecutive mismatches between the spacer sequence and the AAVS1 target sequence. FIG. 5D shows percent indels measured for a variant Cas12i2 (SEQ ID NO: 4) at an EMX1 target; each RNA guide was designed to have double, consecutive mismatches between the spacer sequence and the EMX1 target sequence.

FIG. 6A shows an alignment of the direct repeat sequences of SEQ ID NOs: 8-17.

FIG. 6B shows structures of RNA guides described herein. The direct repeat sequences of SEQ ID NOs: 8-17 form stem-loop structures. Spacers are represented as N's.

### DETAILED DESCRIPTION

The present disclosure relates to a novel Cas12i2 nuclease complex and methods of use thereof. In some aspects, a composition comprising a complex having one or more characteristics is described herein. In some aspects, a method of producing the complex is described. In some aspects, a method of delivering a composition comprising the complex is described.

### Compositions

In some aspects, the invention described herein comprises compositions comprising a complex. In some embodiments, the invention comprises a binary complex comprising a Cas12i2 polypeptide and an RNA guide. In some embodiments, the invention comprises a ternary complex comprising a Cas12i2 polypeptide, an RNA guide, and a target sequence. The RNA guide of the complexes of the disclosure comprises a sequence that is substantially complementary to a target sequence, wherein the target sequence is adjacent to a protospacer adjacent moiety (PAM) sequence of the disclosure.

In some aspects, a binary complex of the invention comprises a Cas12i2 polypeptide and an RNA guide, wherein the RNA guide comprises a sequence that is substantially complementary to a target sequence and the target sequence is adjacent to a PAM sequence of the disclosure, which comprises 4 nucleotides. In some aspects, a ternary complex of the invention comprises such a binary complex bound to the target sequence.

The target sequence of the above aspects is adjacent to a PAM sequence of the disclosure as described herein. The PAM sequence may be immediately adjacent to the target sequence or, for example, within a small number (e.g., 1, 2, 3, 4, or 5) of nucleotides of the target sequence. Further, in the case of a double-stranded target, the RNA guide binds to a first strand of the target (i.e., the target strand or the spacer-complementary strand) and a PAM sequence as described herein is present in the second, complementary strand (i.e., the non-target strand or the non-spacer-complementary strand). In such a case, the PAM sequence is immediately adjacent to (or within a small number, e.g., 1, 2, 3, 4, or 5 nucleotides of) a sequence in the second strand that is complementary to the sequence in the first strand to which the binding moiety binds.

In some embodiments, a PAM sequence of the disclosure comprises the sequence 5′-NTTN-3′, wherein N is any nucleotide. In some embodiments, a PAM sequence of the disclosure comprises the sequence 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein N is any nucleotide, Y is C or T, B is any nucleotide except for A, D is any nucleotide except for C, and R is A or G. In some embodiments, a PAM sequence of the disclosure comprises the sequence 5′-CTTT-3′, 5′-CTTC-3′, 5′-GTTT-3′, 5′-GTTC-3′, 5′-TTTC-3′, 5′-GTTA-3′, or 5′-GTTG-3′.

In some embodiments, a PAM sequence of the disclosure comprises the sequence 5′-TTY-3′ or 5′-TTB-3′, wherein Y is C or T, and B is G, T, or C. In other embodiments, a PAM sequence of the disclosure consists of two nucleotides: 5′-NN-3′, wherein N is any nucleotide (e.g., A, T, C, or G), for example, 5′-TT-3′.

In some embodiments, a PAM sequence of the disclosure comprises the sequence 5′-NTTN-3′, 5′-TTY-3′, or 5′-TTB-3′ (as noted above) and, optionally, one or more (e.g., 1-10) additional N's on the 5′ end. Accordingly, a PAM sequence of the disclosure may comprise the sequence 5′-NTTN-3′, 5′-TTY-3′, or 5′-TTB-3′ with 1, 2, 3, 4, 5, 6, 7, 8, 9, or 10 additional N's on the 5′ end. In some embodiments, each N is independently selected from A, T, C, or G. In some embodiments, each N is independently selected from Y (C or T), B (any nucleotide except for A), and D (any nucleotide except for C). In some embodiments, a PAM sequence of the disclosure does not comprise the sequence, 5′-NXXN-3′, wherein X is any nucleotide except for T, e.g., 5′-NVVN-3′, wherein V is A, G, or C.

Cas12i2 Polypeptide

In some embodiments, the composition of the present invention includes a Cas12i2 polypeptide described herein. In some embodiments, the Cas12i2 polypeptide is encoded by a polynucleotide that comprises a nucleotide sequence such as SEQ ID NO: 1 or comprises an amino acid sequence such as SEQ ID NO: 2. In some embodiments, the polypeptide of the present invention is a variant of a parent polypeptide, wherein the parent is encoded by a polynucleotide that comprises a nucleotide sequence such as SEQ ID NO: 1 or comprises an amino acid sequence such as SEQ ID NO: 2. See Table 1.

TABLE 1
Cas12i2 sequences.

| SEQ | | |
|---|---|---|
| ID NO | Sequence | Description |
| 1 | ATGAGCAGCGCGATCAAAAGCTACAAGAGCGTTCTGCGTCCGAACGAGCGTAAGAA | Nucleotide |
| | CCAACTGCTGAAAAGCACCATTCAGTGCCTGGAAGACGGTAGCGCGTTCTTTTTCA | sequence |
| | AGATGCTGCAAGGCCTGTTTGGTGGCATCACCCCGGAGATTGTTCGTTTCAGCACC | encoding |
| | GAACAGGAGAAACAGCAACAGGATATCGCGCTGTGGTGCGCGGTTAACTGGTTCCG | parent |
| | TCCGGTGAGCCAAGACAGCCTGACCCACACCATTGCGAGCGATAACCTGGTGGAGA | Cas12i2 |
| | AGTTTGAGGAATACTATGGTGGCACCGCGAGCGACGCGATCAAACAGTACTTCAGC | |
| | GCGAGCATTGGCGAAAGCTACTATTGGAACGACTGCCGTCAACAGTACTATGATCT | |
| | GTGCCGTGAGCTGGGTGTTGAGGTGAGCGACCTGACCCATGATCTGGAGATCCTGT | |
| | GCCGTGAAAAGTGCCTGGCGGTTGCGACCGAGAGCAACCAGAACAACAGCATCATT | |
| | AGCGTTCTGTTTGGCACCGGCGAAAAAGAGGACCGTAGCGTGAAACTGCGTATCAC | |
| | CAAGAAAATTCTGGAGGCGATCAGCAACCTGAAAGAAATCCCGAAGAACGTTGCGC | |
| | CGATTCAAGAGATCATTCTGAACGTGGCGAAAGCGACCAAGGAAACCTTCCGTCAG | |
| | GTGTATGCGGGTAACCTGGGTGCGCCGAGCACCCTGGAGAAATTTATCGCGAAGGA | |
| | CGGCCAAAAAGAGTTCGATCTGAAGAAACTGCAGACCGACCTGAAGAAAGTTATTC | |
| | GTGGTAAAAGCAAGGAGCGTGATTGGTGCTGCCAGGAAGAGCTGCGTAGCTACGTG | |
| | GAGCAAAACACCATCCAGTATGACCTGTGGGCGTGGGGCGAAATGTTCAACAAAGC | |
| | GCACACCGCGCTGAAAATCAAGAGCACCCGTAACTACAACTTTGCGAAGCAACGTC | |
| | TGGAACAGTTCAAAGAGATTCAGAGCCTGAACAACCTGOTGGTTGTGAAGAAGCTG | |
| | AACGACTTTTTCGATAGCGAATTTTTCAGCGGCGAGGAAACCTACACCATCTGCGT | |
| | TCACCATCTGGGTGGCAAGGACCTGAGCAAACTGTATAAGGCGTGGGAGGATGATC | |
| | CGGCGGACCCGGAAAACGCGATTGTGGTTCTGTGCGACGATCTGAAAAACAACTTT | |
| | AAGAAAGAGCCGATCCGTAACATTCTGCGTTAGATCTTCACCATTCGTCAAGAATG | |
| | CAGCGCGCAGGACATCCTGGCGGCGGCGAAGTACAACCAACAGCTGGATCGTTATA | |
| | AAAGCCAAAAGGCGAACCCGAGCGTTCTGGGTAACCAGGGCTTTACCTGGACCAAC | |
| | GCGGTGATCCTGCCGGAGAAGGCGCAGCGTAACGACCGTCCGAACAGCCTGGATCT | |
| | GCGTATTTGGCTGTACCTGAAACTGCGTCACCCGGACGGTCGTTGGAAGAAACACC | |
| | ATATCCCGTTCTACGATACCCGTTTCTTCCAAGAAATTTATGCGGCGGGCAACAGC | |
| | CCGGTTGACACCTGCCAGTTTCGTACCCCGCGTTTCGGTTATCACCTGCCGAAACT | |
| | GACCGATCAGACCGCGATCCGTGTTAACAAGAAACATGTGAAAGCGGCGAAGACCG | |
| | AGGCGCGTATTCGTCTGGCGATCCAACAGGGCACCCTGCCGGTGAGCAACCTGAAG | |
| | ATCACCGAAATTAGCGCGACCATCAACAGCAAAGGTCAAGTGCGTATTCCGGTTAA | |
| | GTTTGACGTGGGTCGTCAAAAAGGCACCCTGCAGATCGGTGACCGTTTCTGCGGCT | |
| | ACGATCAAAACCAGACCGCGAGCCACGCGTATAGCCTGTGGGAAGTGGTTAAAGAG | |
| | GGTCAATACCATAAAGAGCTGGGCTGCTTTGTTCGTTTCATCAGCAGCGGTGACAT | |
| | CGTGAGCATTACCGAGAACCGTGGCAACCAATTTGATCAGCTGAGCTATGAAGGTC | |
| | TGGCGTACCCGCAATATGCGGACTGGCGTAAGAAAGCGAGCAAGTTCGTGAGCCTG | |
| | TGGCAGATCACCAAGAAAAACAAGAAAAAGGAAATCGTGACCGTTGAAGCGAAAGA | |
| | GAAGTTTGACGCGATCTGCAAGTACCAGCCGCGTCTGTATAAATTCAACAAGGAGT | |
| | ACGCGTATCTGCTGCGTGATATTGTTCGTGGCAAAAGCCTGGTGGAACTGCAACAG | |
| | ATTCGTCAAGAGATCTTTCGTTTCATTGAACAGGACTGCGGTGTTACCCGTCTGGG | |
| | CAGCCTGAGCCTGAGCACCCTGGAAACCGTGAAAGCGGTTAAGGGTATCATTTACA | |
| | GCTATTTTAGCACCGCGCTGAACGCGAGCAAGAACAACCCGATCAGCGACGAACAG | |
| | CGTAAAGAGTTTGATCCGGAACTGTTCGCGCTGCTGGAAAAGCTGGAGCTGATTCG | |
| | TACCCGTAAAAAGAAACAAAAAGTGGAACGTATCGCGAACAGCCTGATTCAGACCT | |
| | GCCTGGAGAACAACATCAAGTTCATTCGTGGTGAAGGCGACCTGAGCACCACCAAC | |
| | AACGCGACCAAGAAAAAGGCGAACAGCCGTAGCATGGATTGGTTGGCGCGTGGTGT | |
| | TTTTAACAAAATCCGTCAACTGGCGCCGATGCACAACATTACCCTGTTCGGTTGCG | |
| | GCAGCCTGTACACCAGCCACCAGGACCCGCTGGTGCATCGTAACCCGGATAAAGCG | |
| | ATGAAGTGCCGTTGGGCGGCGATCCCGGTTAAGGACATTGGCGATTGGGTGCTGCG | |
| | TAAGCTGAGCCAAAACCTGCGTGCGAAAAACATCGGCACCGGCGAGTACTATCACC | |
| | AAGGTGTTAAAGAGTTCCTGAGCCATTATGAACTGCAGGACCTGGAGGAAGAGCTG | |
| | CTGAAGTGGCGTAGCGATCGTAAAAGCAACATTCCGTGCTGGGTGCTGCAGAACCG | |
| | TCTGGCGGAGAAGCTGGGCAACAAAGAAGCGGTGGTTTACATCCCGGTTCGTGGTG | |
| | GCCGTATTTATTTTGCGACCCACAAGGTGGCGACCGGTGCGGTGAGCATCGTTTTC | |
| | GACCAAAAACAAGTGTGGGTTTGCAACGCGGATCATGTTGCGGCGGCGAACATCGC | |
| | GCTGACCGTGAAGGGTATTGGCGAACAAAGCAGCGACGAAGAGAACCCGGATGGTA | |
| | GCCGTATCAAACTGCAGCTGACCAGC | |
| 2 | MSSAIKSYKSVLRPNERKNQLLKSTIQCLEDGSAFFFKMLQGLFGGITPEIVRFST | Parent |
| | EQEKQQQDIALWCAVNWFRPVSQDSLTHTIASDNLVEKFEEYYGGTASDAIKQYFS | Cas12i2 |
| | ASIGESYYWNDCRQQYYDLCRELGVEVSDLTHDLEILCREKCLAVATESNQNNSII | amino acid |
| | SVLFGTGEKEDRSVKLRITKKILEAISNLKEIPKNVAPIQEIILNVAKATKETFRQ | sequence |
| | VYAGNLGAPSTLEKFIAKDGQKEFDLKKLQTDLKKVIRGKSKERDWCCQEELRSYV | |
| | EQNTIQYDLWAWGEMFNKAHTALKIKSTRNYNFAKQRLEQFKEIQSLNNLLVVKKL | |
| | NDFFDSEFFSGEETYTICVHHLGGKDLSKLYKAWEDDPADPENAIVVLCDDLKNNF | |
| | KKEPIRNILRYIFTIRQECSAQDILAAAKYNQQLDRYKSQKANPSVLGNQGFTWTN | |
| | AVILPEKAQRNDRPNSLDLRIWLYLKLRHPDGRWKKHHIPFYDTRFFQEIYAAGNS | |
| | PVDTCQFRTPRFGYHLPKLTDQTAIRVNKKHVKAAKTEARIRLAIQQGTLPVSNLK | |
| | ITEISATINSKGQVRIPVKFDVGRQKGTLQIGDRFCGYDQNQTASHAYSLWEVVKE | |
| | GQYHKELGCFVRFISSGDIVSITENRGNQFDQLSYEGLAYPQYADWRKKASKFVSL | |
| | WQITKKNKKKEIVTVEAKEKFDAICKYQPRLYKFNKEYAYLLRDIVRGKSLVELQQ | |
| | IRQEIFRFIEQDCGVTRLGSLSLSTLETVKAVKGIIYSYFSTALNASKNNPISDEQ | |
| | RKEFDPELFALLEKLELIRTRKKKQKVERIANSLIQTCLENNIKFIRGEGDLSTTN | |
| | NATKKKANSRSMDWLARGVFNKIRQLAPMHNITLFGCGSLYTSHQDPLVHRNPDKA | |
| | MKCRWAAIPVKDIGDWVLRKLSQNLRAKNIGTGEYYHQGVKEFLSHYELQDLEEEL | |
| | LKWRSDRKSNIPCWVLQNRLAEKLGNKEAVVYIPVRGGRIYFATHKVATGAVSIVF | |
| | DQKQVWVCNADHVAAANIALTVKGIGEQSSDEENPDGSRIKLQLTS | |
| 3 | MSSAIKSYKS VLRPNERKNQ LLKSTIQCLE DGSAFFFKML QGLFGGITPE | Variant |
| | IVRFSTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID |
| | REKCLAVATE SNQNNSIISV LFGTGEKEDR SVKLRITKKI LEAISNLKEI | NO: 3 of |
| | PKNVAPIQEI ILNVAKATKE TFRQVYAGNL GAPSTLEKFI AKDGQKEFDL | PCT/ |
| | KKLQTDLKKV IRGKSKERDW CCQEELRSYV EQNTIQYDLW AWGEMFNKAH | US2021/ |
| | TALKIKSTRN YNFAKQRLEQ FKEIQSLNNL LVVKKLNDFF DSEFFSGEET | 025257 |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKENKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG RWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGEQSSDE ENPDGSRIKL | |
| | QLTS | |
| 4 | MSSAIKSYKS VLRPNERKNQ LLKSTIQCLE DGSAFFFKML QGLFGGITPE | Variant |
| | IVRFSTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID |
| | REKCLAVATE SNQNNSIISV LFGTGEKEDR SVKLRITKKI LEAISNLKEI | NO: 4 of |
| | PKNVAPIQEI ILNVAKATKE TFRQVYAGNL GAPSTLEKFI AKDGQKEFDL | PCT/ |
| | KKLQTDLKKV IRGKSKERDW CCQEELRSYV EQNTIQYDLW AWGEMFNKAH | US2021/ |
| | TALKIKSTRN YNFAKQRLEQ FKEIQSLNNL LVVKKLNDFF DSEFFSGEET | 025257 |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGEQSSDE ENPDGSRIKL | |
| | QLTS | |
| 5 | MSSAIKSYKS VLRPNERKNQ LLKSTIQCLE DGSAFFFKML QGLFGGITPE | Variant |
| | IVRFSTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID |
| | REKCLAVATE SNQNNSIISV LFGTGEKEDR SVKLRITKKI LEAISNLKEI | NO: 5 of |
| | PKNVAPIQEI ILNVAKATKE TFRQVYAGNL GAPSTLEKFI AKDGQKEFDL | PCT/ |
| | KKLQTDLKKV IRGKSKERDW CCQEELRSYV EQNTIQYDLW AWGEMFNKAH | US2021/ |
| | TALKIKSTRN YNFAKQRLEQ FKEIQSLNNL LVVKKLNDFF DSEFFSGEET | 025257 |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELGCFVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGEQSSDE ENPDGGRIKL | |
| | QLTS | |
| 6 | MSSAIKSYKS VLRPNERKNQ LLKSTIQCLE DGSAFFFKML QGLFGGITPE | Variant |
| | IVRFSTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID |
| | REKCLAVATE SNQNNSIISV LFGTGEKEDR SVKLRITKKI LEAISNLKEI | NO: 495 of |
| | PKNVAPIQEI ILNVAKATKE TFRQVYAGNL GAPSTLEKFI AKDGQKEFDL | PCT/ |
| | KKLQTDLKKV IRGKSKERDW CCQEELRSYV EQNTIQYDLW AWGEMFNKAH | US2021/ |
| | TALKIKSTRN YNFAKQRLEQ FKEIQSLNNL LVVKKLNDFF DSEFFSGEET | 025257 |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELRCRVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLAPMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGRQSSDE ENPDGGRIKL | |
| | QLTS | |
| 7 | MSSAIKSYKS VLRPNERKNQ LLKSTIQCLE DGSAFFFKML QGLFGGITPE | Variant |
| | IVRFSTEQEK QQQDIALWCA VNWFRPVSQD SLTHTIASDN LVEKFEEYYG | Cas12i2 of |
| | GTASDAIKQY FSASIGESYY WNDCRQQYYD LCRELGVEVS DLTHDLEILC | SEQ ID |
| | REKCLAVATE SNQNNSIISV LFGTGEKEDR SVKLRITKKI LEAISNLKEI | NO: 496 of |
| | PKNVAPIQEI ILNVAKATKE TFRQVYAGNL GAPSTLEKFI AKDGQKEFDL | PCT/ |
| | KKLQTDLKKV IRGKSKERDW CCQEELRSYV EQNTIQYDLW AWGEMFNKAH | US2021/ |
| | TALKIKSTRN YNFAKQRLEQ FKEIQSLNNL LVVKKLNDFF DSEFFSGEET | 025257 |
| | YTICVHHLGG KDLSKLYKAW EDDPADPENA IVVLCDDLKN NFKKEPIRNI | |
| | LRYIFTIRQE CSAQDILAAA KYNQQLDRYK SQKANPSVLG NQGFTWTNAV | |
| | ILPEKAQRND RPNSLDLRIW LYLKLRHPDG RWKKHHIPFY DTRFFQEIYA | |
| | AGNSPVDTCQ FRTPRFGYHL PKLTDQTAIR VNKKHVKAAK TEARIRLAIQ | |
| | QGTLPVSNLK ITEISATINS KGQVRIPVKF RVGRQKGTLQ IGDRFCGYDQ | |
| | NQTASHAYSL WEVVKEGQYH KELRCRVRFI SSGDIVSITE NRGNQFDQLS | |
| | YEGLAYPQYA DWRKKASKFV SLWQITKKNK KKEIVTVEAK EKFDAICKYQ | |
| | PRLYKFNKEY AYLLRDIVRG KSLVELQQIR QEIFRFIEQD CGVTRLGSLS | |
| | LSTLETVKAV KGIIYSYFST ALNASKNNPI SDEQRKEFDP ELFALLEKLE | |
| | LIRTRKKKQK VERIANSLIQ TCLENNIKFI RGEGDLSTTN NATKKKANSR | |
| | SMDWLARGVF NKIRQLATMH NITLFGCGSL YTSHQDPLVH RNPDKAMKCR | |
| | WAAIPVKDIG DWVLRKLSQN LRAKNRGTGE YYHQGVKEFL SHYELQDLEE | |
| | ELLKWRSDRK SNIPCWVLQN RLAEKLGNKE AVVYIPVRGG RIYFATHKVA | |
| | TGAVSIVFDQ KQVWVCNADH VAAANIALTG KGIGRQSSDE ENPDGGRIKL | |
| | QLTS | |

A nucleic acid sequence encoding the parent polypeptide described herein may be substantially identical to a reference nucleic acid sequence, e.g., SEQ ID NO: 1. In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., nucleic acid sequence encoding the parent polypeptide, e.g., SEQ ID NO: 1. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions (e.g., within a range of medium to high stringency).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., nucleic acid sequence encoding the parent polypeptide, e.g., SEQ ID NO: 1.

In some embodiments, the Cas12i2 polypeptide of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to SEQ ID NO: 2. In some embodiments, the Cas12i2 polypeptide of the present invention comprises a sequence having greater than 50%, 60%, 65%, 70%, 75%, 80%, 85%, 90%, 91%, 92%, 93%, 94%, 95%, 96%, 97%, 98%, or 99%, but not 100%, identity to SEQ ID NO: 2.

In some embodiments, the present invention describes a Cas12i2 polypeptide having a specified degree of amino acid sequence identity to one or more reference polypeptides, e.g., a parent polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2. Homology or identity can be determined by amino acid sequence alignment, e.g., using a program such as BLAST, ALIGN, or CLUSTAL, as described herein.

In some embodiments, the Cas12i2 polypeptide comprises an alteration at one or more (e.g., several) amino acids of a parent polypeptide, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more are altered.

In some embodiments, the Cas12i2 polypeptide is a variant Cas12i2 polypeptide described in PCT/US2021/025257, which is incorporated by reference in its entirety. In some embodiments, the variant Cas12i2 polypeptide comprises one or more of the amino acid substitutions listed in Table 2 of PCT/US2021/025257. In some embodiments, the Cas12i2 polypeptide is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3 of PCT/US2021/025257. In some embodiments, the Cas12i2 polypeptide is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 4 of PCT/US2021/025257. In some embodiments, the Cas12i2 polypeptide is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 5 of PCT/US2021/025257. In some embodiments, the Cas12i2 polypeptide is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 495 of PCT/US2021/025257. In some embodiments, the Cas12i2 polypeptide is a variant Cas12i2 polypeptide having at least 90%, at least 91%, at least 92%, at least 93%, at least 94%, at least 95%, at least 96%, at least 97%, at least 98%, at least 99%, or 100% sequence identity to the amino acid sequence of SEQ ID NO: 496 of PCT/US2021/025257. In some embodiments, the Cas12i2 polypeptide is a variant Cas12i2 polypeptide having at least 90%, at least 91%, at least 92%, at least 93%, at least 94%, at least 95%, at least 96%, at least 97%, at least 98%, at least 99%, or 100% sequence identity to any one of SEQ ID NOs: 3-146 and 495-512 of PCT/US2021/025257.

In some embodiments, the compositions described herein comprise one or more individual (e.g., 2, 3, 4, 5, 6, 7, 8, 9, or more) variant Cas12i2 polypeptides.

Although the changes described herein may be one or more amino acid changes, changes to the Cas12i2 polypeptide may also be of a structural or substantive nature, such as fusion of polypeptides as amino- and/or carboxyl-terminal extensions. For example, Cas12i2 polypeptide may contain additional peptides, e.g., one or more peptides. Examples of additional peptides may include epitope peptides for labelling, such as a polyhistidine tag (His-tag), Myc, and FLAG. In some embodiments, the Cas12i2 polypeptide described herein can be fused to a detectable moiety such as a fluorescent protein (e.g., green fluorescent protein (GFP) or yellow fluorescent protein (YFP)).

In some embodiments, the Cas12i2 polypeptide comprises at least one (e.g., two, three, four, five, six, or more) nuclear localization signal (NLS). In some embodiments, the Cas12i2 polypeptide comprises at least one (e.g., two, three, four, five, six, or more) nuclear export signal (NES). In some embodiments, the Cas12i2 polypeptide comprises at least one (e.g., two, three, four, five, six, or more) NLS and at least one (e.g., two, three, four, five, six, or more) NES.

In some embodiments, the Cas12i2 polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i2 polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/ and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

RNA Guide

In some embodiments, a composition or complex as described herein comprises a targeting moiety (e.g., an RNA guide) that binds the target nucleic acid and the Cas12i2 polypeptide. The RNA guide can bind a target nucleic acid with specific binding affinity to the target nucleic acid. In some embodiments, a composition described herein comprises two or more targeting moieties, e.g., two or more RNA guides (e.g., 2, 3, 4, 5, 6, 7, 8, 9, or more).

In some embodiments, the RNA guide directs the Cas12i2 polypeptide described herein to a particular nucleic acid sequence. Those skilled in the art reading the below examples of particular kinds of RNA guides will understand that, in some embodiments, an RNA guide is site-specific. That is, in some embodiments, an RNA guide associates specifically with one or more target nucleic acid sequences (e.g., specific DNA or genomic DNA sequences) and not to non-targeted nucleic acid sequences (e.g., non-specific DNA or random sequences). In the case of two or more guides within a composition, the two or more guides may target two or more separate Cas12i2 polypeptides (e.g., Cas12i2 polypeptides having the same or different sequence) as described herein to two or more (e.g., 2, 3, 4, 5, 6, 7, 8, 9, or more) target nucleic acids or two or more (e.g., 2, 3, 4, 5, 6, 7, 8, 9, or more) target loci of a target nucleic acid.

The RNA guide may target (e.g., associate with, be directed to, contact, or bind) one or more nucleotides of a target sequence, e.g., a site-specific sequence or a site-specific target. In some embodiments, the effector nucleoprotein (e.g., Cas12i2 polypeptide plus an RNA guide) is activated upon binding to a target nucleic acid that is complementary to a DNA-targeting sequence in the RNA guide (e.g., a sequence-specific substrate or target nucleic acid).

In some embodiments, an RNA guide comprises a DNA-targeting segment (e.g., spacer) having a length of from about 7 nucleotides to about 100 nucleotides. For example, the spacer can have a length of from about 7 nucleotides to about 80 nucleotides, from about 7 nucleotides to about 50 nucleotides, from about 7 nucleotides to about 40 nucleotides, from about 7 nucleotides to about 30 nucleotides, from about 7 nucleotides to about 25 nucleotides, from about 7 nucleotides to about 20 nucleotides, or from about 7 nucleotides to about 19 nucleotides. For example, the spacer can have a length of from about 7 nucleotides to about 20 nucleotides, from about 7 nucleotides to about 25 nucleotides, from about 7 nucleotides to about 30 nucleotides, from about 7 nucleotides to about 35 nucleotides, from about 7 nucleotides to about 40 nucleotides, from about 7 nucleotides to about 45 nucleotides, from about 7 nucleotides to about 50 nucleotides, from about 7 nucleotides to about 60 nucleotides, from about 7 nucleotides to about 70 nucleotides, from about 7 nucleotides to about 80 nucleotides, from about 7 nucleotides to about 90 nucleotides, from about 7 nucleotides to about 100 nucleotides, from about 10 nucleotides to about 25 nucleotides, from about 10 nucleotides to about 30 nucleotides, from about 10 nucleotides to about 35 nucleotides, from about 10 nucleotides to about 40 nucleotides, from about 10 nucleotides to about 45 nucleotides, from about 10 nucleotides to about 50 nucleotides, from about 10 nucleotides to about 60 nucleotides, from about 10 nucleotides to about 70 nucleotides, from about 10 nucleotides to about 80 nucleotides, from about 10 nucleotides to about 90 nucleotides, or from about 10 nucleotides to about 100 nucleotides.

In some embodiments, the spacer of the RNA guide may be generally designed to have a length of between 7 and 50 nucleotides or between 15 and 35 nucleotides (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides) and be complementary to a specific target nucleic acid sequence. In some embodiments, the RNA guide may be designed to be complementary to a specific DNA strand, e.g., of a genomic locus. In some embodiments, the DNA-targeting sequence is designed to be complementary to a specific DNA strand, e.g., of a genomic locus.

The RNA guide may be substantially identical to a complementary strand of a reference nucleic acid sequence. In some embodiments, the RNA guide comprises a sequence (e.g., spacer) having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target nucleic acid. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide has at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a target nucleic acid.

In some embodiments, nucleotide 1 and nucleotide 2 of the spacer of an RNA guide demonstrate 100% complementarity to the target nucleic acid. In some embodiments, the remaining nucleotides of the spacer, e.g., nucleotide 3 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 3 of the spacer of an RNA guide demonstrate 100% complementarity to the target nucleic acid. In some embodiments, the remaining nucleotides of the spacer, e.g., nucleotide 4 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 4 of the spacer of an RNA guide demonstrate 100% complementarity to the target nucleic acid. In some embodiments, the remaining nucleotides of the spacer, e.g., nucleotide 5 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 5 of the spacer of an RNA guide demonstrate at least 80% complementarity to the target nucleic acid. In some embodiments, nucleotide 1 through nucleotide 5 of the spacer of an RNA guide demonstrate 100% complementarity to the target nucleic acid. In some embodiments, the remaining nucleotides of the spacer, e.g., nucleotide 6 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 6 of the spacer of an RNA guide demonstrate 100% complementarity to the target nucleic acid. In some embodiments, the remaining nucleotides of the spacer, e.g., nucleotide 7 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 7 of the spacer of an RNA guide demonstrate at least 85% complementarity to the target nucleic acid. In some embodiments, nucleotide 1 through nucleotide 7 of the spacer of an RNA guide demonstrate 100% complementarity to the target nucleic acid. In some embodiments, the remaining nucleotides of the spacer, e.g., nucleotide 8 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 8 of the spacer of an RNA guide demonstrate at least 85% (e.g., at least 87%) complementarity to the target nucleic acid. In some embodiments, nucleotide 1 through nucleotide 8 of the spacer of an RNA guide demonstrate 100% complementarity to the target nucleic acid. In some embodiments, the remaining nucleotides of the spacer, e.g., nucleotide 9 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 9 of the spacer of an RNA guide demonstrate at least 85% (e.g., at least 88%) complementarity to the target nucleic acid. In some embodiments, nucleotide 1 through nucleotide 9 of the spacer of an RNA guide demonstrate 100% complementarity to the target nucleic acid. In some embodiments, the remaining nucleotides of the spacer, e.g., nucleotide 10 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 10 of the spacer of an RNA guide demonstrate at least 85% (e.g., at least 90%) complementarity to the target nucleic acid. In some embodiments, nucleotide 1 through nucleotide 10 of the spacer of an RNA guide demonstrate 100% complementarity to the target nucleic acid. In some embodiments, the remaining nucleotides of the spacer, e.g., nucleotide 11 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 11 of the spacer of an RNA guide demonstrate at least 85% (e.g., at least 90%) complementarity to the target nucleic acid. In some embodiments, nucleotide 1 through nucleotide 11 of the spacer of an RNA guide demonstrate 100% complementarity to the target nucleic acid. In some embodiments, the remaining nucleotides of the spacer, e.g., nucleotide 12 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 12 of the spacer of an RNA guide demonstrate at least 85% (e.g., at least 91%) complementarity to the target nucleic acid. In some embodiments, nucleotide 1 through nucleotide 12 of the spacer of an RNA guide demonstrate 100% complementarity to the target nucleic acid. In some embodiments, the remaining nucleotides of the spacer, e.g., nucleotide 13 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 13 of the spacer of an RNA guide demonstrate at least 85% (e.g., at least 92%) complementarity to the target nucleic acid. In some embodiments, nucleotide 1 through nucleotide 13 of the spacer of an RNA guide demonstrate 100% complementarity to the target nucleic acid. In some embodiments, the remaining nucleotides of the spacer, e.g., nucleotide 14 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 14 of the spacer of an RNA guide demonstrate at least 85% (e.g., at least 92%) complementarity to the target nucleic acid. In some embodiments, nucleotide 1 through nucleotide 14 of the spacer of an RNA guide demonstrate 100% complementarity to the target nucleic acid. In some embodiments, the remaining nucleotides of the spacer, e.g., nucleotide 15 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 15 of the spacer of an RNA guide demonstrate at least 85% (e.g., at least 93%) complementarity to the target nucleic acid. In some embodiments, nucleotide 1 through nucleotide 15 of the spacer of an RNA guide demonstrate 100% complementarity to the target nucleic acid. In some embodiments, the remaining nucleotides of the spacer, e.g., nucleotide 16 through nucleotide 20, 21, 22, 23, 24, 25, 26, 27, 28, 29, 30, 31, 32, 33, 34, or 35, demonstrate at least 85% (e.g., at least 85%, at least 90%, at least 95%, or 100%) complementarity to the target nucleic acid.

In some embodiments, nucleotide 1 through nucleotide 16 of the spacer of an RNA guide demonstrate at least 80% (e.g., at least 85%, at least 90%, or at least 95%) complementarity to the target nucleic acid. In some embodiments, nucleotide 1 through nucleotide 16 of the spacer of an RNA guide demonstrate 100% complementarity to the target nucleic acid. In some embodiments, the remaining nucleotides of the spacer, e.g., nucleotide 17 through nucleotide 20, 21, 22, 23, 24, 25, 26, 27, 28, 29, 30, 31, 32, 33, 34, or 35, demonstrate at least 60% (e.g., at least 65%, at least 70%, at least 75%, at least 80%, at least 85%, at least 90%, at least 95%, or 100%) complementarity to the target nucleic acid.

In some embodiments, nucleotide 1 through nucleotide 17 of the spacer of an RNA guide demonstrate at least 80% (e.g., at least 85%, at least 90%, or at least 95%) complementarity to the target nucleic acid. In some embodiments, nucleotide 1 through nucleotide 17 of the spacer of an RNA guide demonstrate 100% complementarity to the target nucleic acid. In some embodiments, the remaining nucleotides of the spacer, e.g., nucleotide 18 through nucleotide 20, 21, 22, 23, 24, 25, 26, 27, 28, 29, 30, 31, 32, 33, 34, or 35, demonstrate at least 60% (e.g., at least 65%, at least 70%, at least 75%, at least 80%, at least 85%, at least 90%, at least 95%, or 100%) complementarity to the target nucleic acid.

In some embodiments, nucleotide 1 through nucleotide 18 of the spacer of an RNA guide demonstrate at least 80% (e.g., at least 85%, at least 90%, or at least 95%) complementarity to the target nucleic acid. In some embodiments, nucleotide 1 through nucleotide 18 of the spacer of an RNA guide demonstrate 100% complementarity to the target nucleic acid. In some embodiments, the remaining nucleotides of the spacer, e.g., nucleotide 19 through nucleotide 20, 21, 22, 23, 24, 25, 26, 27, 28, 29, 30, 31, 32, 33, 34, or 35, demonstrate at least 60% (e.g., at least 65%, at least 70%, at least 75%, at least 80%, at least 85%, at least 90%, at least 95%, or 100%) complementarity to the target nucleic acid.

In some embodiments, nucleotide 1 through nucleotide 19 of the spacer of an RNA guide demonstrate at least 80% (e.g., at least 85%, at least 90%, or at least 95%) complementarity to the target nucleic acid. In some embodiments, nucleotide 1 through nucleotide 19 of the spacer of an RNA guide demonstrate 100% complementarity to the target nucleic acid. In some embodiments, the remaining nucleotides of the spacer, e.g., nucleotide 20 through nucleotide 21, 22, 23, 24, 25, 26, 27, 28, 29, 30, 31, 32, 33, 34, or 35, demonstrate at least 60% (e.g., at least 65%, at least 70%, at least 75%, at least 80%, at least 85%, at least 90%, at least 95%, or 100%) complementarity to the target nucleic acid.

In some embodiments, nucleotide 1 through nucleotide 20 of the spacer of an RNA guide demonstrate at least 80% (e.g., at least 85%, at least 90%, or at least 95%) complementarity to the target nucleic acid. In some embodiments, nucleotide 1 through nucleotide 20 of the spacer of an RNA guide demonstrate 100% complementarity to the target nucleic acid. In some embodiments wherein the spacer is longer than 20 nucleotides, the remaining nucleotides of the spacer, e.g., nucleotide 21 through nucleotide 22, 23, 24, 25, 26, 27, 28, 29, 30, 31, 32, 33, 34, or 35, demonstrate at least 60% (e.g., at least 65%, at least 70%, at least 75%, at least 80%, at least 85%, at least 90%, at least 95%, or 100%) complementarity to the target nucleic acid.

In some embodiments, the nucleotides of the spacer that bind adjacent to the PAM sequence of the present invention (e.g., the 5′ portion of the spacer) is substantially complementary to the target nucleic acid. In some embodiments, the nucleotides of the spacer that contact the PAM-distal region of the target nucleic acid (e.g., the 3′ portion of the spacer) has at least about 60%, at least about 65%, at least about 70%, at least about 75%, at least about 80%, at least about 85%, at least about 90%, at least about 95%, or 100% complementarity to the target nucleic acid.

In certain embodiments, the RNA guide includes, consists essentially of, or comprises a direct repeat sequence linked to a DNA-targeting sequence. In some embodiments, the RNA guide includes a direct repeat sequence and a DNA-targeting sequence or a direct repeat-DNA-targeting sequence-direct repeat sequence. In some embodiments, the RNA guide includes a truncated direct repeat sequence and a DNA-targeting sequence, which is typical of processed or mature crRNA. In some embodiments, the Cas12i2 polypeptide described herein forms a complex with the RNA guide, and the RNA guide directs the complex to associate with site-specific target nucleic acid that is complementary to at least a portion of the RNA guide.

In some embodiments, the direct repeat sequence is a sequence of Table 2 or a portion of a sequence of Table 2. The direct repeat sequence can comprise nucleotide 1 through nucleotide 36 of any one of SEQ ID NOs: 8, 9, 10, 11, 12, 13, 14, or 15. The direct repeat sequence can comprise nucleotide 2 through nucleotide 36 of any one of SEQ ID NOs: 8, 9, 10, 11, 12, 13, 14, or 15. The direct repeat sequence can comprise nucleotide 3 through nucleotide 36 of any one of SEQ ID NOs: 8, 9, 10, 11, 12, 13, 14, or 15. The direct repeat sequence can comprise nucleotide 4 through nucleotide 36 of any one of SEQ ID NOs: 8, 9, 10, 11, 12, 13, 14, or 15. The direct repeat sequence can comprise nucleotide 5 through nucleotide 36 of any one of SEQ ID NOs: 8, 9, 10, 11, 12, 13, 14, or 15. The direct repeat sequence can comprise nucleotide 6 through nucleotide 36 of any one of SEQ ID NOs: 8, 9, 10, 11, 12, 13, 14, or 15. The direct repeat sequence can comprise nucleotide 7 through nucleotide 36 of any one of SEQ ID NOs: 8, 9, 10, 11, 12, 13, 14, or 15. The direct repeat sequence can comprise nucleotide 8 through nucleotide 36 of any one of SEQ ID NOs: 8, 9, 10, 11, 12, 13, 14, or 15. The direct repeat sequence can comprise nucleotide 9 through nucleotide 36 of any one of SEQ ID NOs: 8, 9, 10, 11, 12, 13, 14, or 15. The direct repeat sequence can comprise nucleotide 10 through nucleotide 36 of any one of SEQ ID NOs: 8, 9, 10, 11, 12, 13, 14, or 15. The direct repeat sequence can comprise nucleotide 11 through nucleotide 36 of any one of SEQ ID NOs: 8, 9, 10, 11, 12, 13, 14, or 15. The direct repeat sequence can comprise nucleotide 12 through nucleotide 36 of any one of SEQ ID NOs: 8, 9, 10, 11, 12, 13, 14, or 15. The direct repeat sequence can comprise nucleotide 13 through nucleotide 36 of any one of SEQ ID NOs: 8, 9, 10, 11, 12, 13, 14, or 15. The direct repeat sequence can comprise nucleotide 14 through nucleotide 36 of any one of SEQ ID NOs: 8, 9, 10, 11, 12, 13, 14, or 15. The direct repeat sequence can comprise nucleotide 1 through nucleotide 34 of SEQ ID NO: 16. The direct repeat sequence can comprise nucleotide 2 through nucleotide 34 of SEQ ID NO: 16. The direct repeat sequence can comprise nucleotide 3 through nucleotide 34 of SEQ ID NO: 16. The direct repeat sequence can comprise nucleotide 4 through nucleotide 34 of SEQ ID NO: 16. The direct repeat sequence can comprise nucleotide 5 through nucleotide 34 of SEQ ID NO: 16. The direct repeat sequence can comprise nucleotide 6 through nucleotide 34 of SEQ ID NO: 16. The direct repeat sequence can comprise nucleotide 7 through nucleotide 34 of SEQ ID NO: 16. The direct repeat sequence can comprise nucleotide 8 through nucleotide 34 of SEQ ID NO: 16. The direct repeat sequence can comprise nucleotide 9 through nucleotide 34 of SEQ ID NO: 16. The direct repeat sequence can comprise nucleotide 10 through nucleotide 34 of SEQ ID NO: 16. The direct repeat sequence can comprise nucleotide 11 through nucleotide 34 of SEQ ID NO: 16. The direct repeat sequence can comprise nucleotide 12 through nucleotide 34 of SEQ ID NO: 16. In some embodiments, the direct repeat sequence is set forth in SEQ ID NO: 17. In some embodiments, the direct repeat sequence comprises a portion of the sequence set forth in SEQ ID NO: 17.

In some embodiments, the direct repeat sequence has at least 90% identity (e.g., at least 90%, 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 8, 9, 10, 11, 12, 13, 14, or 15. The direct repeat sequence can have at least 90% identity to a sequence comprising 2 through nucleotide 36 of any one of SEQ ID NOs: 8, 9, 10, 11, 12, 13, 14, or 15. The direct repeat sequence can have at least 90% identity to a sequence comprising 3 through nucleotide 36 of any one of SEQ ID NOs: 8, 9, 10, 11, 12, 13, 14, or 15. The direct repeat sequence can have at least 90% identity to a sequence comprising 4 through nucleotide 36 of any one of SEQ ID NOs: 8, 9, 10, 11, 12, 13, 14, or 15. The direct repeat sequence can have at least 90% identity to a sequence comprising 5 through nucleotide 36 of any one of SEQ ID NOs: 8, 9, 10, 11, 12, 13, 14, or 15. The direct repeat sequence can have at least 90% identity to a sequence comprising 6 through nucleotide 36 of any one of SEQ ID NOs: 8, 9, 10, 11, 12, 13, 14, or 15. The direct repeat sequence can have at least 90% identity to a sequence comprising 7 through nucleotide 36 of any one of SEQ ID NOs: 8, 9, 10, 11, 12, 13, 14, or 15. The direct repeat sequence can have at least 90% identity to a sequence comprising 8 through nucleotide 36 of any one of SEQ ID NOs: 8, 9, 10, 11, 12, 13, 14, or 15. The direct repeat sequence can have at least 90% identity to a sequence comprising 9 through nucleotide 36 of any one of SEQ ID NOs: 8, 9, 10, 11, 12, 13, 14, or 15. The direct repeat sequence can have at least 90% identity to a sequence comprising 10 through nucleotide 36 of any one of SEQ ID NOs: 8, 9, 10, 11, 12, 13, 14, or 15. The direct repeat sequence can have at least 90% identity to a sequence comprising 11 through nucleotide 36 of any one of SEQ ID NOs: 8, 9, 10, 11, 12, 13, 14, or 15. The direct repeat sequence can have at least 90% identity to a sequence comprising 12 through nucleotide 36 of any one of SEQ ID NOs: 8, 9, 10, 11, 12, 13, 14, or 15. The direct repeat sequence can have at least 90% identity to a sequence comprising 13 through nucleotide 36 of any one of SEQ ID NOs: 8, 9, 10, 11, 12, 13, 14, or 15. The direct repeat sequence can have at least 90% identity to a sequence comprising 14 through nucleotide 36 of any one of SEQ ID NOs: 8, 9, 10, 11, 12, 13, 14, or 15. The direct repeat sequence can have at least 90% identity to a sequence comprising 1 through nucleotide 34 of SEQ ID NO: 16. The direct repeat sequence can have at least 90% identity to a sequence comprising 2 through nucleotide 34 of SEQ ID NO: 16. The direct repeat sequence can have at least 90% identity to a sequence comprising 3 through nucleotide 34 of SEQ ID NO: 16. The direct repeat sequence can have at least 90% identity to a sequence comprising 4 through nucleotide 34 of SEQ ID NO: 16. The direct repeat sequence can have at least 90% identity to a sequence comprising 5 through nucleotide 34 of SEQ ID NO: 16. The direct repeat sequence can have at least 90% identity to a sequence comprising 6 through nucleotide 34 of SEQ ID NO: 16. The direct repeat sequence can have at least 90% identity to a sequence comprising 7 through nucleotide 34 of SEQ ID NO: 16. The direct repeat sequence can have at least 90% identity to a sequence comprising 8 through nucleotide 34 of SEQ ID NO: 16. The direct repeat sequence can have at least 90% identity to a sequence comprising 9 through nucleotide 34 of SEQ ID NO: 16. The direct repeat sequence can have at least 90% identity to a sequence comprising 10 through nucleotide 34 of SEQ ID NO: 16. The direct repeat sequence can have at least 90% identity to a sequence comprising 11 through nucleotide 34 of SEQ ID NO: 16. The direct repeat sequence can have at least 90% identity to a sequence comprising 12 through nucleotide 34 of SEQ ID NO: 16. In some embodiments, the direct repeat sequence has at least 90% identity (e.g., at least 90%, 91%, 92%, 93%, 94%, 95%, 96%, 97%, 98%, 99%, or 100% identity) to SEQ ID NO: 17. In some embodiments, the direct repeat sequence has at least 90% identity (e.g., at least 90%, 91%, 92%, 93%, 94%, 95%, 96%, 97%, 98%, 99%, or 100% identity) to a portion of the sequence set forth in SEQ ID NO: 17.

In some embodiments, the direct repeat sequence is at least 90% identical (e.g., at least 90%, 91%, 92%, 93%, 94%, 95%, 96%, 97%, 98%, 99%, or 100% identical) to the reverse complement of any one of SEQ ID NOs: 8-17. In some embodiments, the direct repeat sequence is the reverse complement of any one of SEQ ID NOs: 8-17.

TABLE 2
Direct repeat sequences

| Sequence | |
|---|---|
| identifier | DR Sequence |
| SEQ ID NO: 8 | GUUGCAAAACCCAAGAAAUCCGUCUUUCAUUGACGG |
| SEQ ID NO: 9 | AAUAGCGGCCCUAAGAAAUCCGUCUUUCAUUGACGG |
| SEQ ID NO: 10 | AUUGGAACUGGCGAGAAAUCCGUCUUUCAUUGACGG |
| SEQ ID NO: 11 | CCAGCAACACCUAAGAAAUCCGUCUUUCAUUGACGG |
| SEQ ID NO: 12 | CGGCGCUCGAAUAGGAAAUCCGUCUUUCAUUGACGG |
| SEQ ID NO: 13 | GUGGCAACACCUAAGAAAUCCGUCUUUCAUUGACGG |
| SEQ ID NO: 14 | GUUGCAACACCUAAGAAAUCCGUCUUUCAUUGACGG |
| SEQ ID NO: 15 | GUUGCAAUGCCUAAGAAAUCCGUCUUUCAUUGACGG |
| SEQ ID NO: 16 | GCAACACCUAAGAAAUCCGUCUUUCAUUGACGGG |
| SEQ ID NO: 17 | AGAAAUCCGUCUUUCAUUGACGG |

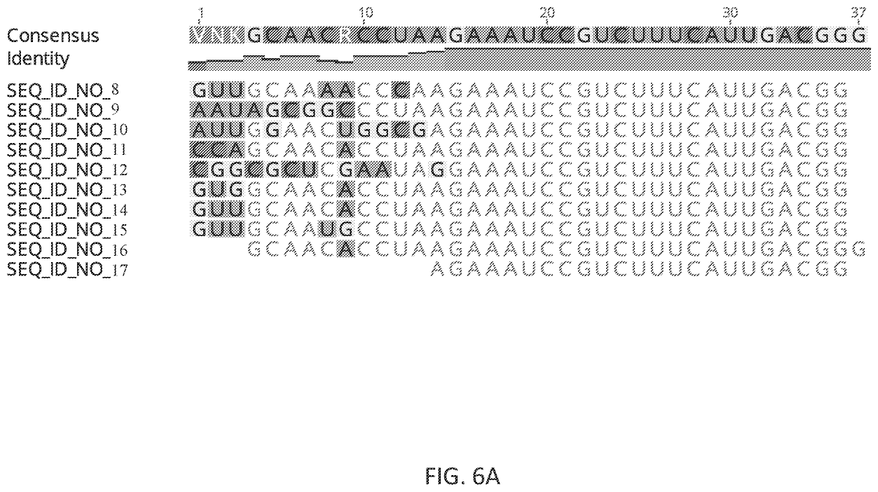

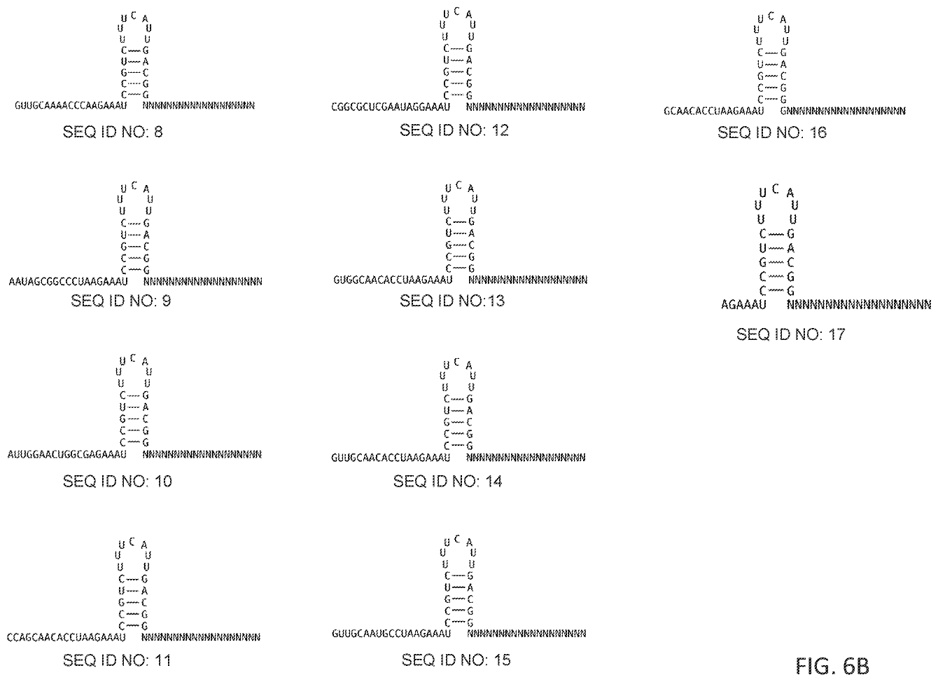

The direct repeat sequences of Table 2 were aligned, as shown in FIG. 6A. In some embodiments, the direct repeat sequence comprises a sequence having at least 90% identity (e.g., at least 90%, 91%, 92%, 93%, 94%, 95%, 96%, 97%, 98%, 99%, or 100% identity) to X₁GAAAUCCGUCUUUCAUUGACGG (SEQ ID NO: 18), wherein X₁ is A or G. In some embodiments, the direct repeat sequence comprises a sequence having at least 95% identity (e.g., at least 95%, 96%, 97%, 98%, 99%, or 100% identity) to SEQ ID NO: 18. In some embodiments, the direct repeat sequence comprises the sequence of SEQ ID NO: 18.

In some embodiments, the direct repeat sequence is at least 90% identical (e.g., at least 90%, 91%, 92%, 93%, 94%, 95%, 96%, 97%, 98%, 99%, or 100% identical) to SEQ ID NO: 18 or a portion of SEQ ID NO: 18. In some embodiments, the direct repeat sequence is at least 95% identical (e.g., at least 95%, 96%, 97%, 98%, 99%, or 100% identical) to SEQ ID NO: 18 or a portion of SEQ ID NO: 18. In some embodiments, the direct repeat sequence comprises the sequence of SEQ ID NO: 18 or a portion of SEQ ID NO: 18.

In some embodiments, the direct repeat sequence comprises a sequence having at least 90% identity (e.g., at least 90%, 91%, 92%, 93%, 94%, 95%, 96%, 97%, 98%, 99%, or 100% identity) to a sequence set forth as 5′-CCGUCUUUCAUUGACGG-3′ (SEQ ID NO: 19), a sequence having at least 95% identity (e.g., at least 95%, 96%, 97%, 98%, 99%, or 100% identity) to SEQ ID NO: 19), or a sequence set forth as SEQ ID NO: 19.

In some embodiments, the direct repeat sequence comprises a first portion comprising nucleotides 1-13 of SEQ ID NO: 17 or a sequence having at least 90% identity (e.g., at least 90%, at least 95%, or 100% identity) to nucleotides 1-13 of SEQ ID NO: 17, a second portion comprising nucleotides 14-23 of SEQ ID NO: 17 or a sequence having at least 90% identity (e.g., at least 90%, at least 95%, or 100% identity) to nucleotides 14-23, and a heterologous sequence disposed between the first portion and the second portion. In some embodiments, the heterologous sequence a nucleic acid of between 1 nucleotide and about 200 nucleotides. In some embodiments, the heterologous sequence is a DNA sequence, an RNA sequence, or a DNA/RNA hybrid sequence. In some embodiments, the heterologous sequence is an aptamer.

In some embodiments, the direct repeat sequence comprises a first portion comprising nucleotides 1-14 of SEQ ID NO: 17 or a sequence having at least 90% identity e.g., at least 90%, at least 95%, or 100% identity) to nucleotides 1-14 of SEQ ID NO: 17, a second portion comprising nucleotides 15-23 of SEQ ID NO: 17 or a sequence having at least 90% identity e.g., at least 90%, at least 95%, or 100% identity) to nucleotides 15-23, and a heterologous sequence disposed between the first portion and the second portion. In some embodiments, the heterologous sequence a nucleic acid of between 1 nucleotide and about 200 nucleotides. In some embodiments, the heterologous sequence is a DNA sequence, an RNA sequence, or a DNA/RNA hybrid sequence. In some embodiments, the heterologous sequence is an aptamer.

In some embodiments, the direct repeat sequence comprises a first portion comprising nucleotides 1-15 of SEQ ID NO: 17 or a sequence having at least 90% identity e.g., at least 90%, at least 95%, or 100% identity) to nucleotides 1-15 of SEQ ID NO: 17, a second portion comprising nucleotides 16-23 of SEQ ID NO: 17 or a sequence having at least 90% identity e.g., at least 90%, at least 95%, or 100% identity) to nucleotides 16-23, and a heterologous sequence disposed between the first portion and the second portion. In some embodiments, the heterologous sequence a nucleic acid of between 1 nucleotide and about 200 nucleotides. In some embodiments, the heterologous sequence is a DNA sequence, an RNA sequence, or a DNA/RNA hybrid sequence. In some embodiments, the heterologous sequence is an aptamer.

In some embodiments, the direct repeat sequence comprises a first portion comprising nucleotides 1-16 of SEQ ID NO: 17 or a sequence having at least 90% identity e.g., at least 90%, at least 95%, or 100% identity) to nucleotides 1-16 of SEQ ID NO: 17, a second portion comprising nucleotides 17-23 of SEQ ID NO: 17 or a sequence having at least 90% identity e.g., at least 90%, at least 95%, or 100% identity) to nucleotides 17-23, and a heterologous sequence disposed between the first portion and the second portion. In some embodiments, the heterologous sequence a nucleic acid of between 1 nucleotide and about 200 nucleotides. In some embodiments, the heterologous sequence is a DNA sequence, an RNA sequence, or a DNA/RNA hybrid sequence. In some embodiments, the heterologous sequence is an aptamer.

Structures of the direct repeat sequences of Table 2 are shown in FIG. 6B. In some embodiments, the direct repeat sequences described herein comprise a stem-loop structure proximal to a 3′ end of the direct repeat sequence. In some embodiments, the direct repeat comprises a first stem nucleotide strand 5 nucleotides in length and a second stem nucleotide strand 5 nucleotides in length. In some embodiments, the first and second stem nucleotide strands are complementary to each other. In some embodiments, the direct repeat comprises a loop between the first stem nucleotide strand and the second stem nucleotide strand. In some embodiments, the loop comprises 7 nucleotides.

In some embodiments, the direct repeat comprises a first stem nucleotide strand 4 nucleotides in length and a second stem nucleotide strand 4 nucleotides in length. In some embodiments, the first and second stem nucleotide strands are complementary to each other. In some embodiments, the direct repeat comprises a loop between the first stem nucleotide strand and the second stem nucleotide strand. In some embodiments, the loop comprises 7 to 9 nucleotides.

In some embodiments, the direct repeat comprises a first stem nucleotide strand 3 nucleotides in length and a second stem nucleotide strand 3 nucleotides in length. In some embodiments, the first and second stem nucleotide strands are complementary to each other. In some embodiments, the direct repeat comprises a loop between the first stem nucleotide strand and the second stem nucleotide strand. In some embodiments, the loop comprises 7 to 11 nucleotides.

In some embodiments, in the direct repeat sequence of SEQ ID NO: 17, nucleotide 7 is complementary to (e.g., binds to) nucleotide 23, nucleotide 8 is complementary to (e.g., binds to) nucleotide 22, nucleotide 9 is complementary to (e.g., binds to) nucleotide 21, nucleotide 10 is complementary to (e.g., binds to) nucleotide 21, nucleotide 11 is complementary to (e.g., binds to) nucleotide 20, and nucleotide 12 is complementary to (e.g., binds to) nucleotide 19.

In some embodiments, in a direct repeat sequence having at least 90% identity) e.g., at least 90%, at least 95%, or 100% identity) to SEQ ID NO: 17, at least three nucleotides of the first stem nucleotide strand are complementary to nucleotides of the second stem nucleotide strand. In some embodiments, at least 3 of the following are complementary to one another: (a) nucleotide 7 and nucleotide 23, (b) nucleotide 8 and nucleotide 22, (c) nucleotide 9 and nucleotide 21, (d) nucleotide 10 and nucleotide 20, and (e) nucleotide 11 and nucleotide 19. In some embodiments, in a direct repeat sequence having at least 95% identity (e.g., at least 95%, 96%, 97%, 98%, 99%, or 100% identity) to SEQ ID NO: 17, at least three nucleotides of the first stem nucleotide strand are complementary to nucleotides of the second stem nucleotide strand. In some embodiments, at least 3 of the following are complementary to one another: (a) nucleotide 7 and nucleotide 23, (b) nucleotide 8 and nucleotide 22, (c) nucleotide 9 and nucleotide 21, (d) nucleotide 10 and nucleotide 20, and (e) nucleotide 11 and nucleotide 19.

In some embodiments, a spacer sequence of an RNA guide described herein comprises a 3′ sequence. In some embodiments, the 3′ sequence does not bind to the target sequence. In some embodiments, a portion of the 3′ sequence forms a loop structure. The loop can comprise about 1 nucleotide to about 10 nucleotides (e.g., about 1, 2, 3, 4, 5, 6, 7, 8, 9, or 10 nucleotides). In some embodiments, a portion of the 3′ sequence binds to the spacer sequence (e.g., a portion of 3′ sequence comprises complementarity to the spacer sequence.) In some embodiments, a portion of the 3′ sequence is at least 80% complementary to a portion of the spacer sequence.

In some embodiments, an RNA guide described herein comprises a uracil (U). In some embodiments, an RNA guide described herein comprises a thymine (T). In some embodiments, a direct repeat sequence of an RNA guide described herein comprises a uracil (U). In some embodiments, a direct repeat sequence of an RNA guide described herein comprises a thymine (T). In some embodiments, a direct repeat sequence described herein comprises a uracil in one or more places indicated as thymine herein. In some embodiments, a direct repeat sequence described herein comprises a thymine in one or more places indicated as uracil herein.

In some embodiments, the RNA guide has an architecture similar to, for example International Publication Nos. WO 2014/093622 and WO 2015/070083, the entire contents of each of which are incorporated herein by reference.

Modifications

The RNA guide or any of the nucleic acid sequences encoding the Cas12i2 polypeptide may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide or any of the nucleic acid sequences encoding components of the Cas12i2 polypeptides may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O(1-thiophosphate)-cytidine (α-thio-cytidine), 5′-O-(1-thiophosphate)-guano sine, 5′-O-(1-thiophosphate)-uridine, or 5′-O(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonylcarbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

Target Sequence

The methods disclosed herein are applicable for a variety of target sequences. In some embodiments, the target sequence is a DNA molecule, such as a DNA locus (referred to herein as a target sequence or an on-target sequence). In some embodiments, the target sequence is an RNA, such as an RNA locus or mRNA. In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, a single-stranded target sequence does not require a PAM sequence.

The target sequence may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. The target sequence may also comprise any sequence. In some embodiments, the target sequence is GC-rich, such as having at least about any one of 40%, 45%, 50%, 55%, 60%, 65%, or higher GC content. In some embodiments, the target sequence has a GC content of at least about 70%, 80%, or more. In some embodiments, the target sequence is a GC-rich fragment in a non-GC-rich target sequence. In some embodiments, the target sequence is not GC-rich. In some embodiments, the target sequence has one or more secondary structures or higher-order structures. In some embodiments, the target sequence is not in a condensed state, such as in a chromatin, to render the target sequence inaccessible by the binary complex so that the binary complex can form a ternary complex.

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc. In some embodiments, the target sequence is a non-coding gene, such as transposon, miRNA, tRNA, ribosomal RNA, ribozyme, or lincRNA. In some embodiments, the target sequence is a plasmid.

In some embodiments, the target sequence is exogenous to a cell. In some embodiments, the target sequence is a viral nucleic acid, such as viral DNA or viral RNA. In some embodiments, the target sequence is a horizontally transferred plasmid. In some embodiments, the target sequence is integrated in the genome of the cell. In some embodiments, the target sequence is not integrated in the genome of the cell. In some embodiments, the target sequence is a plasmid in the cell. In some embodiments, the target sequence is present in an extrachromosomal array.

In some embodiments, the target sequence is an isolated nucleic acid, such as an isolated DNA or an isolated RNA. In some embodiments, the target sequence is present in a cell-free environment. In some embodiments, the target sequence is an isolated vector, such as a plasmid. In some embodiments, the target sequence is an ultrapure plasmid.

The target sequence is a segment of the target sequence that hybridizes to the RNA guide. In some embodiments, the target sequence is cleaved by the binary complex. In some embodiments, the target sequence has only one copy of the target sequence. In some embodiments, the target sequence has more than one copy, such as at least about any one of 2, 3, 4, 5, 10, 100, or more copies of the target sequence. For example, a target sequence comprising a repeated sequence in a genome of a viral nucleic acid or a bacterium may be targeted by the Cas12i2 nucleoprotein.

The target sequence is adjacent to a PAM sequence of the disclosure as described herein. The PAM sequence may be immediately adjacent to the target sequence or, for example, within a small number (e.g., 1, 2, 3, 4, or 5) of nucleotides of the target sequence. In the case of a double-stranded target, the RNA guide binds to a first strand of the target and a PAM sequence as described herein is present in the second, complementary strand. In such a case, the PAM sequence is immediately adjacent to (or within a small number, e.g., 1, 2, 3, 4, or 5 nucleotides of) a sequence in the second strand that is complementary to the sequence in the first strand to which the binding moiety binds.

A PAM sequence of the disclosure can comprise four nucleotides and, in some embodiments, a PAM sequence of the disclosure comprises the sequence 5′-NTTN-3′ wherein N is any nucleotide (e.g., A, G, T, or C). In other embodiments, a PAM sequence of the disclosure comprises the sequence 5′-TTY-3′ or 5′-TTB-3′, wherein Y is C or T, and B is G, T, or C. In other embodiments, a PAM sequence of the disclosure consists of two nucleotides: 5′-NN-3′, wherein N is any nucleotide (e.g., A, T, C, or G), for example, 5′-TT-3′. A PAM sequence of the disclosure can optionally include one or more (e.g., 1, 2, 3, 4, 5, 6, 7, 8, 9, or 10) additional nucleotides 5′ of the sequence 5′-NTTN-3′, 5′-TTY-3′, or 5′-TTB-3′. These one or more additional nucleotides can be N's and thus can each be any nucleotide (e.g., A, G, T, or C) or a subset thereof (e.g., R (G or A), Y (C or T), K (G or T), M (A or C), S (G or C), W (A or T), B (G, T, or C), D (G, A, or T), H (A, C, or T), or V (G, C, or A)). A PAM sequence of the disclosure can also consist of 2 nucleotides. Examples of PAM sequences of the disclosure are set forth in Table 3.

TABLE 3
Exemplary PAM sequences or PAM core sequences

| | | | |
|---|---|---|---|
| 5′-NNNN-3′ | 5′-NTTY-3′ | 5′-CTTY-3′ | 5′-CTTT-3′ |
| 5′-NTTN-3′ | 5′-NTTC-3′ | 5′-CTTR-3′ | 5′-CTTC-3′ |
| 5′-NN-3′ | 5′-NTTT-3′ | 5′-DTTT-3′ | 5′-GTTT-3′ |
| 5′-TT-3′ | 5′-NTTB-3′ | 5′-DTTR-3′ | 5′-GTTC-3′ |
| 5′-TTY-3′ | 5′-NTTG-3′ | 5′-ATTN-3′ | 5′-TTTC-3′ |
| 5′-TTB-3′ | 5′-NTTA-3′ | 5′-GTTN-3′ | 5′-GTTA-3′ |
| | | | 5′-GTTG-3′ |

In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene. In some embodiments, wherein the target sequence is exogenous to a cell, the target sequence comprises a sequence that is not found in the genome of the cell.

Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target sequence that is complementary to and hybridizes with the RNA guide is referred to as the “complementary strand” and the strand of the target sequence that is complementary to the “complementary strand” (and is therefore not complementary to the RNA guide) is referred to as the “noncomplementary strand” or “non-complementary strand”.

Complex

Described herein are compositions and methods relating to a complex, e.g., a binary complex and/or a ternary complex. Generally, the Cas12i2 polypeptide and the RNA guide bind to each other in a molar ratio of about 1:1 to form a binary complex. As used herein, binding of the Cas12i2 polypeptide and the RNA guide to form the binary complex is referred to herein as loading the RNA guide to the polypeptide. Generally, a Cas12i2 polypeptide, an RNA guide, and a target sequence associate with each other in a molar ratio of about 1:1:1 to form the ternary complex. Generally, the binary complex, e.g., the Cas12i2 polypeptide and the RNA guide, binds to a target sequence in a molar ratio of about 1:1 to form the ternary complex. As used herein, binding of the binary complex to the target sequence to form the ternary complex is referred to herein as loading the binary complex to the target sequence. In some embodiments, the ternary complex follows a one-binary complex rule, i.e., the binary complex does not dissociate from the bound target sequence (e.g., target DNA substrate) or switch the target sequence with a free, unbound nucleic acid (e.g., non-target sequence).

In some embodiments, the binary complex exhibits ternary complex formation at a target sequence adjacent to a PAM sequence of the disclosure at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the binary complex exhibits ternary complex formation at a target sequence adjacent to a PAM sequence of the disclosure over a range of temperatures, from about 20° C. to about 65° C.

In some embodiments, the binary complex exhibits ternary complex formation at a target sequence adjacent to a PAM sequence of the disclosure at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, the binary complex exhibits ternary complex formation at a target sequence adjacent to a PAM sequence of the disclosure over a range of incubation times.

In some embodiments, the binary complex exhibits ternary complex formation at a target sequence adjacent to a PAM sequence of the disclosure in a buffer having a pH in a range of about 7.3 to 8.6. In one embodiment, the binary complex exhibits ternary complex formation at a target sequence adjacent to a PAM sequence of the disclosure in a pH of about 7.3, 7.4, 7.5, 7.6, 7.7, 7.8, 7.9, 8.0, 8.1, 8.2, 8.3, 8.4, 8.5, or 8.6.

In some aspects, the binary complex exhibits increased ternary complex formation with a target sequence adjacent to a PAM sequence of the disclosure, as compared to a target sequence that is not adjacent to a PAM sequence of the disclosure. In some embodiments, the binary complex exhibits increased ternary complex formation with a target sequence adjacent to a PAM sequence of the disclosure over a range of temperatures, such as from about 20° C. to about 65° C. In some embodiments, the binary complex exhibits increased ternary complex formation with a target sequence adjacent to a PAM sequence of the disclosure over a range of incubation times. In some embodiments, the binary complex exhibits increased ternary complex formation with a target sequence adjacent to a PAM sequence of the disclosure over a range of pH values, such as from about 7.3 to about 8.6. Ternary complex formation with a target sequence can be increased by at least about 4%-200%.

In some aspects, the binary complex demonstrates increased on-target binding to a target sequence adjacent to a PAM sequence of the disclosure, as compared to a target sequence that is not adjacent to a PAM sequence of the disclosure. In some embodiments, the binary complex exhibits increased on-target binding to a target sequence adjacent to a PAM sequence of the disclosure over a range of temperatures, such as from about 20° C. to about 65° C. In some embodiments, the binary complex exhibits increased on-target binding to a target sequence adjacent to a PAM sequence of the disclosure over a range of incubation times. In some embodiments, the binary complex exhibits increased on-target binding to a target sequence adjacent to a PAM sequence of the disclosure over a range of pH values, such as from about 7.3 to about 8.6. On-target binding can be increased by at least about 4%-200%.

In some aspects, the binary complex demonstrates increased binding affinity to a target sequence adjacent to a PAM sequence of the disclosure, as compared to a target sequence that is not adjacent to a PAM sequence of the disclosure. In some embodiments, the binary complex exhibits increased binding affinity to a target sequence adjacent to a PAM sequence of the disclosure over a range of temperatures, such as from about 20° C. to about 65° C. In some embodiments, the binary complex exhibits increased binding affinity to a target sequence adjacent to a PAM sequence ofthe disclosure over a range of incubation times. In some embodiments, the binary complex exhibits increased binding affinity to a target sequence adjacent to a PAM sequence of the disclosure over a range of pH values, such as from about 7.3 to about 8.6. Binding affinity to a target sequence can be increased by at least about 4%-200%.

In some aspects, the binary complex demonstrates increased RNA-DNA interactions with a target sequence adjacent to a PAM sequence of the disclosure, as compared to a target sequence that is not adjacent to a PAM sequence of the disclosure. In some embodiments, the binary complex exhibits increased RNA-DNA interactions with a target sequence adjacent to a PAM sequence of the disclosure over a range of temperatures, such as from about 20° C. to about 65° C. In some embodiments, the binary complex exhibits increased RNA-DNA interactions with a target sequence adjacent to a PAM sequence of the disclosure over a range of incubation times. In some embodiments, the binary complex exhibits increased RNA-DNA interactions with a target sequence adjacent to a PAM sequence of the disclosure over a range of pH values, such as from about 7.3 to about 8.6. RNA-DNA interactions can be increased by at least about 4%-200%.

In some aspects, the binary complex demonstrates decreased dissociation from a target sequence adjacent to a PAM sequence of the disclosure, as compared to a target sequence that is not adjacent to a PAM sequence of the disclosure. In some embodiments, the binary complex exhibits decreased dissociation from a target sequence adjacent to a PAM sequence of the disclosure over a range of temperatures, such as from about 20° C. to about 65° C. In some embodiments, the binary complex exhibits decreased dissociation from a target sequence adjacent to a PAM sequence of the disclosure over a range of incubation times. In some embodiments, the binary complex exhibits decreased dissociation from a target sequence adjacent to a PAM sequence of the disclosure over a range of pH values, such as from about 7.3 to about 8.6. Dissociation from a target sequence can be decreased from at least about 4%-100%.

In some aspects, the binary complex demonstrates increased activity when bound to a target sequence adjacent to a PAM sequence of the disclosure, as compared to a sequence that is not adjacent to a PAM sequence of the disclosure. In some embodiments, the binary complex exhibits increased activity when bound to a target sequence adjacent to a PAM sequence of the disclosure over a range of temperatures, such as from about 20° C. to about 65° C. In some embodiments, the binary complex exhibits increased activity when bound to a target sequence adjacent to a PAM sequence of the disclosure over a range of incubation times. In some embodiments, the binary complex exhibits increased activity when bound to a target sequence adjacent to a PAM sequence of the disclosure over a range of pH values, such as from about 7.3 to about 8.6.

In some aspects wherein the binary complex exhibits increased ternary complex formation with a target sequence adjacent to a PAM sequence of the disclosure, the binary complex exhibits decreased ternary complex formation with a non-target sequence. The non-target sequence may be adjacent to a PAM sequence of the disclosure, adjacent to a different PAM sequence, or not adjacent to a PAM. Ternary complex formation with a non-target sequence can be decreased by at least about 4%-100%.

In some aspects wherein the binary complex demonstrates increased on-target binding to a target sequence adjacent to a PAM sequence of the disclosure, the binary complex exhibits decreased off-target binding to a non-target sequence. The non-target sequence may be adjacent to a PAM sequence of the disclosure, adjacent to a different PAM sequence, or not adjacent to a PAM sequence. Off-target binding can be decreased by at least about 4%-100%.

In some aspects wherein the binary complex demonstrates increased binding affinity to a target sequence adjacent to a PAM sequence of the disclosure, the binary complex exhibits decreased binding affinity to a non-target sequence (e.g., off-target sequence). The non-target sequence may be adjacent to a PAM sequence of the disclosure, adjacent to a different PAM sequence, or not adjacent to a PAM sequence. Binding affinity to a non-target sequence can be decreased by at least about 4%-100%.

In some aspects wherein the binary complex demonstrates increased RNA-DNA interactions with a target sequence adjacent to a PAM sequence of the disclosure, the binary complex exhibits decreased RNA-DNA interactions with a non-target sequence (e.g., off-target sequence). The non-target sequence may be adjacent to a PAM sequence of the disclosure, adjacent to a different PAM sequence, or not adjacent to a PAM sequence. RNA-DNA interactions can be decreased by at least about 4%-100%.

In some embodiments, increased binding affinity or binding of a binary complex to a target sequence adjacent to a PAM sequence of the disclosure is associated with increased activity of the binary complex at the target sequence (e.g., on-target activity) adjacent to a PAM sequence of the disclosure. In some embodiments, increased binding affinity or binding of a binary complex to a target sequence adjacent to a PAM sequence of the disclosure results in increased activity of the binary complex at the target sequence (e.g., on-target activity) adjacent to a PAM sequence of the disclosure.

In some embodiments, decreased binding affinity or binding of a binary complex to a non-target sequence is associated with decreased activity of the binary complex at a non-target sequence (e.g., off-target activity). In some embodiments, decreased binding affinity or binding of a binary complex to a non-target sequence results in decreased activity of the binary complex at the non-target sequence (e.g., off-target activity). The non-target sequence may be adjacent to a PAM sequence of the disclosure, adjacent to a different PAM sequence, or not adjacent to a PAM sequence.

In some embodiments, decreased binding affinity or binding of a binary complex to a non-target sequence is associated with increased activity of the binary complex at a target sequence (e.g., on-target activity) adjacent to a PAM sequence of the disclosure. In some embodiments, decreased binding affinity or binding of a binary complex to a non-target sequence results in increased activity of the binary complex at a target sequence (e.g., on-target activity) adjacent to a PAM sequence of the disclosure. The non-target sequence may be adjacent to a PAM sequence of the disclosure, adjacent to a different PAM sequence, or not adjacent to a PAM sequence.

In some embodiments of these aspects, the target is a double-stranded target. The RNA guide in these embodiments binds to a first strand of the target (i.e., the target strand or the spacer-complementary strand), and a PAM sequence as described herein is present in the second, complementary strand (i.e., the non-target strand or the non-spacer-complementary strand). In such a case, the PAM sequence is immediately adjacent to (or within a small number, e.g., 1, 2, 3, 4, or 5 nucleotides of) a sequence in the second strand that is complementary to the sequence in the first strand to which the binding moiety binds.

### Preparation

In some embodiments, the Cas12i2 polypeptide of the present invention can be prepared by (a) culturing bacteria which produce the Cas12i2 polypeptide of the present invention, isolating the Cas12i2 polypeptide, optionally, purifying the Cas12i2 polypeptide, and complexing the Cas12i2 polypeptide with RNA guide. The Cas12i2 polypeptide can be also prepared by (b) a known genetic engineering technique, specifically, by isolating a gene encoding the Cas12i2 polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the Cas12i2 polypeptide can be prepared by (c) an in vitro coupled transcription-translation system and then complexes with RNA guide. Bacteria that can be used for preparation of the Cas12i2 polypeptide of the present invention are not particularly limited as long as they can produce the Cas12i2 polypeptide of the present invention. Some nonlimiting examples of the bacteria include E. coli cells described herein.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

Vectors

The present invention provides a vector for expressing the Cas12i2 polypeptide described herein or nucleic acids encoding the described herein may be incorporated into a vector. In some embodiments, a vector of the invention includes a nucleotide sequence encoding Cas12i2 polypeptide. In some embodiments, a vector of the invention includes a nucleotide sequence encoding the Cas12i2 polypeptide.

The present invention also provides a vector that may be used for preparation of the Cas12i2 polypeptide or compositions comprising the Cas12i2 polypeptide as described herein. In some embodiments, the invention includes the composition or vector described herein in a cell. In some embodiments, the invention includes a method of expressing the composition comprising the Cas12i2 polypeptide, or vector or nucleic acid encoding the Cas12i2 polypeptide, in a cell. The method may comprise the steps of providing the composition, e.g., vector or nucleic acid, and delivering the composition to the cell.

Expression of natural or synthetic polynucleotides is typically achieved by operably linking a polynucleotide encoding the gene of interest, e.g., nucleotide sequence encoding the Cas12i2 polypeptide, to a promoter and incorporating the construct into an expression vector. The expression vector is not particularly limited as long as it includes a polynucleotide encoding the Cas12i2 polypeptide of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the Cas12i2 polypeptide from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the Cas12i2 polypeptide of the present invention has been transferred into the host cells and then expressed without fail.

The preparation method for recombinant expression vectors is not particularly limited, and examples thereof include methods using a plasmid, a phage or a cosmid.

Methods of Expression

The present invention includes a method for protein expression, comprising translating the Cas12i2 polypeptide described herein.

In some embodiments, a host cell described herein is used to express the Cas12i2 polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i2 polypeptide. After expression of the Cas12i2 polypeptide, the host cells can be collected and Cas12i2 polypeptide purified from the cultures etc. according to conventional methods (for example, filtration, centrifugation, cell disruption, gel filtration chromatography, ion exchange chromatography, etc.).

In some embodiments, the methods for Cas12i2 polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i2 polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i2 polypeptide.

A variety of methods can be used to determine the level of production of a mature Cas12i2 polypeptide in a host cell. Such methods include, but are not limited to, for example, methods that utilize either polyclonal or monoclonal antibodies specific for the Cas12i2 polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the Cas12i2 polypeptide in a cell, comprising providing a polyribonucleotide encoding the Cas12i2 polypeptide to a host cell wherein the polyribonucleotide encodes the Cas12i2 polypeptide, expressing the Cas12i2 polypeptide in the cell, and obtaining the Cas12i2 polypeptide from the cell.

Introduction of Alteration or Mutation

Nucleic acid sequences encoding Cas12i2 polypeptides may be generated by synthetic methods known in the art. Using the nucleic acid sequence encoding the parent polypeptide itself as a framework, alternations or mutations can be inserted one or more at a time to alter the nucleic acid sequence encoding the parent polypeptide. Along the same lines, the parent polypeptide may be altered or mutated by introducing the changes into the polypeptide sequence as it is synthetically synthesized. This may be accomplished by methods well known in the art.

The production and introduction of alteration or mutation into a parent polypeptide sequence can be accomplished using any methods known by those of skill in the art. In particular, in some embodiments, oligonucleotide primers for PCR may be used for the rapid synthesis of a DNA template including the one or more alterations or mutations in the nucleic acid sequence encoding for the variant polypeptide. Site-specific mutagenesis may also be used as a technique useful in the preparation of individual peptides, or biologically functional equivalent proteins or peptides, through specific mutagenesis of the underlying DNA. The technique further provides a ready ability to prepare and test variants, incorporating one or more of the foregoing considerations, by introducing one or more nucleotide sequence changes into the DNA. Site-specific mutagenesis allows the production of variants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

Introduction of structural variations, such as fusion of polypeptides as amino- and/or carboxyl-terminal extensions can be accomplished in a similar fashion as introduction of alterations or mutations into the parent polypeptide. The additional peptides may be added to the parent polypeptide or variant polypeptide by including the appropriate nucleic acid sequence encoding the additional peptides to the nucleic acid sequence encoding the parent polypeptide or variant polypeptide. Optionally, the additional peptides may be appended directly to the variant polypeptide through synthetic polypeptide production.

Binary Complexing

In some embodiments, the Cas12i2 polypeptide can be overexpressed in a host cell and purified as described herein, then complexed with an RNA guide (e.g., in a test tube) to form, e.g., a Cas12i2 ribonucleoprotein (RNP) (e.g., binary complex).

In some embodiments, complexation of a binary complex occurs at a temperature lower than about any one of 20° C., 21° C., 22° C., 23° C., 24° C., 25° C., 26° C., 27° C., 28° C., 29° C., 30° C., 31° C., 32° C., 33° C., 34° C. 35° C., 36° C., 37° C., 38° C., 39° C., 40° C., 41° C., 42° C., 43° C., 44° C., 45° C., 50° C., or 55° C. In some embodiments, the Cas12i2 polypeptide does not dissociate from the RNA guide or bind to a free RNA at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, after binary complex formation, a Cas12i2 ribonucleoprotein complex does not exchange the RNA guide with a different RNA.

In some embodiments, the Cas12i2 polypeptide and RNA guide are complexed in a binary complexation buffer. In some embodiments, the Cas12i2 polypeptide is stored in a buffer that is replaced with a binary complexation buffer to form a complex with the RNA guide. In some embodiments, the Cas12i2 polypeptide is stored in a binary complexation buffer.

In some embodiments, the binary complexation buffer has a pH in a range of about 7.3 to 8.6. In one embodiment, the pH of the binary complexation buffer is about 7.3. In one embodiment, the pH of the binary complexation buffer is about 7.4. In one embodiment, the pH of the binary complexation buffer is about 7.5. In one embodiment, the pH of the binary complexation buffer is about 7.6. In one embodiment, the pH of the binary complexation buffer is about 7.7. In one embodiment, the pH of the binary complexation buffer is about 7.8. In one embodiment, the pH of the binary complexation buffer is about 7.9. In one embodiment, the pH of the binary complexation buffer is about 8.0. In one embodiment, the pH of the binary complexation buffer is about 8.1. In one embodiment, the pH of the binary complexation buffer is about 8.2. In one embodiment, the pH of the binary complexation buffer is about 8.3. In one embodiment, the pH of the binary complexation buffer is about 8.4. In one embodiment, the pH of the binary complexation buffer is about 8.5. In one embodiment, the pH of the binary complexation buffer is about 8.6.

In some embodiments, the Cas12i2 polypeptide can be overexpressed and complexed with the RNA guide in a host cell prior to purification as described herein. In some embodiments, mRNA or DNA encoding the Cas12i2 polypeptide is introduced into a cell so that the Cas12i2 polypeptide is expressed in the cell. The RNA guide, which guides the Cas12i2 polypeptide to the desired target sequence is also introduced into the cell, whether simultaneously, separately or sequentially from a single mRNA or DNA construct, such that the necessary ribonucleoprotein complex is formed in the cell.

Ternary Complexing

In some embodiments, the Cas12i2 polypeptide, RNA guide, and target sequence, as described herein, form a ternary complex (e.g., in a test tube or cell).

In some embodiments, the binary complex (e.g., complex of Cas12i2 polypeptide and RNA guide) as described herein, is further complexed with the target sequence (e.g., in a test tube or cell) to form a ternary complex.

The invention provides methods for targeting a sequence adjacent to a PAM of the disclosure. These methods can include contacting a target sequence (e.g., a target sequence in a cell) with a binary complex as described herein, wherein the target sequence is adjacent to a PAM of the disclosure, as described herein, and binding of the binary complex to the target sequence forms a ternary complex as described herein. In the case of target sequences in a cell, the binary complex can optionally be introduced into the cell in the form of polypeptide and RNA, or by use of a vector encoding one or more of the components of the binary complex.

The invention also provides methods for designing RNA guides for use in targeting a target sequence, wherein the target sequence is adjacent to a PAM of the disclosure, as described herein. According to these methods, once a target sequence is selected for targeting, the sequence of the target sequence is analyzed for the presence of a PAM of the disclosure, as described herein. Once one or more of such PAMs are identified in the target sequence, then one or more RNA guides are designed to bind to a target sequence adjacent to the one or more PAM, as described herein. In the case of an RNA guide, for example, a DNA-targeting sequence having substantial complementarity to the target sequence, adjacent to the PAM of the disclosure, is designed as described above, e.g., in the “RNA Guide” section.

In the ternary complexes, the binding moiety (e.g., RNA guide) is bound to a target sequence adjacent to (e.g., immediately adjacent to or within 1, 2, 3, 4, or 5 nucleotides of) a PAM sequence of the disclosure. The PAM may be immediately adjacent to the target sequence or, for example, within a small number (e.g., 1, 2, 3, 4, or 5) of nucleotides of the target sequence. In the case of a double-stranded target, the RNA guide binds to a first strand of the target and a PAM sequence as described herein is present in the second, complementary strand. In such a case, the PAM sequence is immediately adjacent to (or within a small number, e.g., 1, 2, 3, 4, or 5 nucleotides of) a sequence in the second strand that is complementary to the sequence in the first strand to which the binding moiety binds.

As described herein, a PAM sequence of the disclosure comprises the sequence 5′-NTTN-3′, wherein N is any nucleotide. In some embodiments, a PAM sequence of the disclosure comprises the sequence 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein N is any nucleotide, Y is C or T, B is any nucleotide except for A, D is any nucleotide except for C, and R is A or G. In some embodiments, a PAM sequence of the disclosure comprises the sequence 5′-CTTT-3′, 5′-CTTC-3′, 5′-GTTT-3′, 5′-GTTC-3′, 5′-TTTC-3′, 5′-GTTA-3′, or 5′-GTTG-3′.

In some embodiments, a PAM sequence of the disclosure comprises the sequence 5′-TTY-3′ or 5′-TTB-3′, wherein Y is C or T, and B is G, T, or C. In other embodiments, a PAM sequence of the disclosure consists of two nucleotides: 5′-NN-3′, wherein N is any nucleotide (e.g., A, T, C, or G), for example, 5′-TT-3′.

In some embodiments, a PAM sequence of the disclosure comprises the sequence 5′-NTTN-3′, 5′-TTY-3′, or 5′-TTB-3′ (as noted above) and, optionally, one or more (e.g., 1-10) additional N's on the 5′ end. Accordingly, a PAM sequence of the disclosure may comprise the sequence 5′-NTTN-3′, 5′-TTY-3′, or 5′-TTB-3′ with 1, 2, 3, 4, 5, 6, 7, 8, 9, or 10 additional N's on the 5′ end. In some embodiments, each N is independently selected from A, T, C, or G. In some embodiments, each N is independently selected from Y (C or T), B (any nucleotide except for A), and D (any nucleotide except for C). In some embodiments, a PAM sequence of the disclosure does not comprise the sequence, 5′-NXXN-3′, wherein X is any nucleotide except for T, e.g., 5′-NVVN-3′, wherein V is A, G, or C.

In some embodiments, complexation of the ternary complex occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the binary complex does not dissociate from the target sequence or bind to a free nucleic acid (e.g., free DNA) at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, after ternary complex formation, a binary complex does not exchange the target sequence with a different nucleic acid.

In some embodiments, the Cas12i2 polypeptide, RNA guide, and target sequence are complexed in a ternary complexation buffer. In some embodiments, the Cas12i2 polypeptide is stored in a buffer that is replaced with a ternary complexation buffer to form a complex with the RNA guide and target sequence. In some embodiments, the Cas12i2 polypeptide is stored in a ternary complexation buffer.

In some embodiments, the binary complex and target sequence are complexed in a ternary complexation buffer. In some embodiments, the binary complex is stored in a buffer that is replaced with a ternary complexation buffer to form a complex with the target sequence. In some embodiments, the binary complex is stored in a ternary complexation buffer.

In some embodiments, the ternary complexation buffer has a pH in a range of about 7.3 to 8.6. In one embodiment, the pH of the ternary complexation buffer is about 7.3. In one embodiment, the pH of the ternary complexation buffer is about 7.4. In one embodiment, the pH of the ternary complexation buffer is about 7.5. In one embodiment, the pH of the ternary complexation buffer is about 7.6. In one embodiment, the pH of the ternary complexation buffer is about 7.7. In one embodiment, the pH of the ternary complexation buffer is about 7.8. In one embodiment, the pH of the ternary complexation buffer is about 7.9. In one embodiment, the pH of the ternary complexation buffer is about 8.0. In one embodiment, the pH of the ternary complexation buffer is about 8.1. In one embodiment, the pH of the ternary complexation buffer is about 8.2. In one embodiment, the pH of the ternary complexation buffer is about 8.3. In one embodiment, the pH of the ternary complexation buffer is about 8.4. In one embodiment, the pH of the ternary complexation buffer is about 8.5. In one embodiment, the pH of the ternary complexation buffer is about 8.6.

### Delivery

Compositions or complexes described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i2 polypeptide, RNA guide, donor DNA, etc.), one or more transcripts thereof, and/or a pre-formed Cas12i2 polypeptide/RNA guide complex (i.e., binary complex) to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

Cells

Compositions or complexes described herein may be delivered to a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the bacterial cell is not related to the bacterial species from which the parent polypeptide is derived. In some embodiments, the cell is an archaeal cell or derived from an archaeal cell. In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as Ago-coding vector and gDNA) or Ago-gDNA complex described herein is used to establish a new cell line comprising one or more vector-derived sequences to establish a new cell line comprising modification to the target nucleic acid. In some embodiments, cells transiently or non-transiently transfected with one or more nucleic acids (such as Cas12i2 polypeptide-encoding vector and RNA guide) or Cas12i2 polypeptide/RNA guide complex (i.e., binary complex) described herein, or cell lines derived from such cells are used in assessing one or more test compounds.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In some embodiments, the Cas12i2 polypeptide has nuclease activity that induces double-stranded breaks or single-stranded breaks in a target nucleic acid, (e.g. genomic DNA). The double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides into the target nucleic acid. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleaved target nucleic acid. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, the cell culture is synchronized to enhance the efficiency of the methods. In some embodiments, cells in S and G2 phases are used for HDR-mediated gene editing. In some embodiments, the cell can be subjected to the method at any cell cycle. In some embodiments, cell over-plating significantly reduces the efficacy of the method. In some embodiments, the method is applied to a cell culture at no more than about any one of 40%, 45%, 50%, 55%, 60%, 65%, or 70% confluency.

In some embodiments, binding of the Cas12i2 polypeptide/RNA guide complex (i.e., binary complex) to the target nucleic acid in the cell recruits one or more endogenous cellular molecules or pathways other than DNA repair pathways to modify the target nucleic acid. In some embodiments, binding of the binary complex blocks access of one or more endogenous cellular molecules or pathways to the target nucleic acid, thereby modifying the target nucleic acid. For example, binding of the binary complex may block endogenous transcription or translation machinery to decrease the expression of the target nucleic acid.

In some embodiments, delivery of a Cas12i2 polypeptide does not substantially affect viability of the cell. In some embodiments, a cell remains viable following delivery of a Cas12i2 polypeptide. In some embodiments, a cell remains viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 polypeptide. In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a Cas12i2 polypeptide. In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 polypeptide. In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a Cas12i2 polypeptide. In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 polypeptide. In some embodiments, at least 90% (e.g., 90%, 91%, 92%, 93%, 94%, 95%, 96%, 97%, 98%, 99%, or 100%) of a plurality of cells remain viable following delivery of a Cas12i2 polypeptide. In some embodiments, at least 90% (e.g., 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 polypeptide.

In some embodiments, delivery of a Cas12i2 binary complex (e.g., RNP) does not substantially affect viability of the cell. In some embodiments, a cell remains viable following delivery of a Cas12i2 binary complex (e.g., RNP). In some embodiments, a cell remains viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 binary complex (e.g., RNP). In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a Cas12i2 binary complex (e.g., RNP). In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 binary complex (e.g., RNP). In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a Cas12i2 binary complex (e.g., RNP). In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 binary complex (e.g., RNP). In some embodiments, at least 90% (e.g., 90%, 91%, 92%, 93%, 94%, 95%, 96%, 97%, 98%, 99%, or 100%) of a plurality of cells remain viable following delivery of a Cas12i2 binary complex (e.g., RNP). In some embodiments, at least 90% (e.g., 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a Cas12i2 binary complex (e.g., RNP).

### Kits

The invention also provides kits or systems that can be used, for example, to carry out a method described herein. In some embodiments, the kits or systems include a Cas12i2 polypeptide, for example, a variant Cas12i2 polypeptide. In some embodiments, the kits or systems include a polynucleotide that encodes such a Cas12i2 polypeptide, and optionally the polynucleotide is comprised within a vector, e.g., as described herein. The kits or systems also can include an RNA guide, such as an RNA guide, e.g., as described herein. The RNA guide of the kits or systems of the invention can be designed to target a sequence of interest, as is known in the art. The Cas12i2 polypeptide and the RNA guide can be packaged within the same vial or other vessel within a kit or system or can be packaged in separate vials or other vessels, the contents of which can be mixed prior to use. The kits or systems can additionally include, optionally, a buffer and/or instructions for use of the Cas12i2 and/or RNA guide.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Determination of PAM Sequences for Cas12i2 System in E. coli

In this Example, Cas12i2 PAM sequences were determined in an E. coli system.

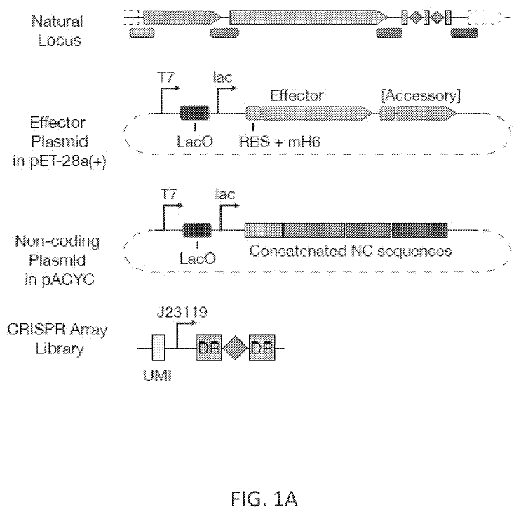

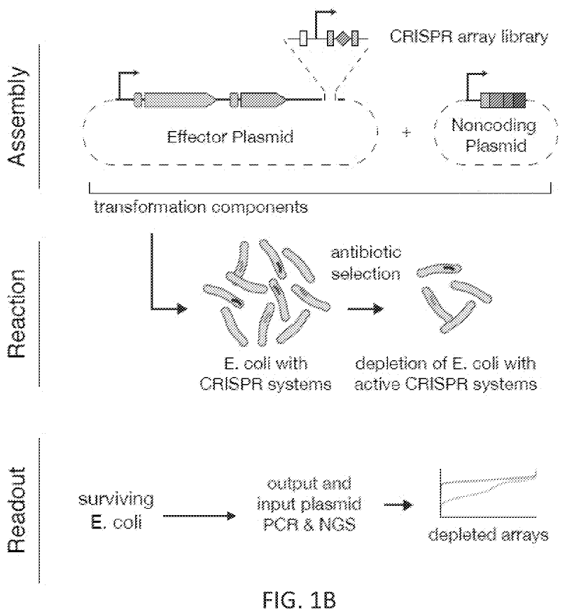

Cas12i2 (SEQ ID NO: 3) was E. coli codon-optimized, synthesized (Genscript) and cloned into a custom expression system derived from pET-28a(+) (EMD-Millipore) to create the Cas12i2 plasmid. The Cas12i2 plasmid included a nucleic acid encoding Cas12i2 under the control of a lac promoter, an E. coli ribosome binding sequence, and an acceptor site for a CRISPR array library driven by a J23119 promoter following the open reading frame for Cas12i2. Noncoding sequences flanking Cas genes (including 150 nucleotides of terminal CDS coding sequence) or the CRISPR array were synthesized (Genscript) and cloned into pACYC184 (New England Biolabs) to create the non-coding plasmid. See FIG. 1A.

An oligonucleotide library synthesis (OLS) pool containing direct repeat-spacer-direct repeat sequences was computationally designed, where the direct repeat represents a consensus direct repeat sequence found in the CRISPR array associated with the natural Cas locus, and the spacer represents a sequence tiling the pACYC184 plasmid (the non-coding plasmid) comprising chloramphenicol and tetracycline resistance genes, E. coli essential genes, or a negative control sequence (GFP). The direct repeat sequence in each library for Cas12i2 was the sequence: GTTGCAAAACCCAAGAAATCCGTCTTTCATTGACGG (SEQ ID NO: 20). The spacer length was determined by the mode of the spacer lengths found in the endogenous CRISPR array. Redundant direct repeat sequences were represented in the library that tile the pACYC184 plasmid (the non-coding plasmid), E. coli essential genes, or negative control sequence to provide internal controls. An individual direct repeat-spacer-direct repeat sequence is also described as a CRISPR array in these Examples.

The library of targeting CRISPR array sequences was next cloned into the Cas12i2 plasmid to create a Cas12i2/CRISPR array library. Flanking restriction sites, a unique molecular identifier (barcode), unique PCR priming sites for specific amplification of the targeting library from the larger pool, and a J23119 promoter were appended to the targeting library using PCR (NEBNext High-Fidelity 2×PCR Master Mix), and then an optimized restriction enzyme and ligase (New England Biolabs) was added to generate the Cas12i2/CRISPR array library. This represented the input library for the screen.

Next, E. coli were co-transformed with the Cas12i2/CRISPR array library and the non-coding plasmid. The cells were electroporated with the input library according to the manufacturer's protocols using an electroporation system (Bio-rad) with a 1.0 mm cuvette. The cells were plated onto bioassay plates with both chloramphenicol (Fisher) and kanamycin (Alfa Aesar) and grown for 11 hours. Subsequently, the approximate colony count was estimated to ensure sufficient library representation, and the cells were harvested. See FIG. 1B.

Cells transformed with Cas12i2/CRISPR array library were grown, harvested, and analyzed. Plasmid DNA fractions were extracted from the harvested cells to create the output library using a DNA prep kit (Qiagen), while total RNA was harvested by processing the harvested cells with an RNA purification kit (Zymo Research), followed by extraction using an RNA prep kit (Zymo Research).

Activity of the engineered Cas12i2/CRISPR array library in E. coli was assessed, wherein bacterial cell death was used as the proxy for Cas12i2 activity. An active Cas12i2 enzyme associated with a CRISPR array sequence could selectively bind and disrupt expression of a spacer sequence target, e.g., pACYC184 plasmid or E. coli essential gene, resulting in cell death, thereby depleting representation of this specific CRISPR array in the output library, as opposed to the input library.

A next generation sequencing (NGS) library for detecting those CRISPR arrays depleted from the output library, as compared to the input library, was prepared by performing PCR on both the input and output libraries, using the unique primers that flank the targeting library of the CRISPR array to identify each CRISPR array sequence by the barcodes. The library was then normalized, pooled, and loaded onto a high-throughput sequence system (Illumina) to evaluate the presence (and absence) of barcodes.

NGS data for screening input and output libraries were demultiplexed using software to convert base call files into FASTQ files. Reads for each sample included information about the targeting library in the screening For each sample, the total number of reads for each CRISPR array sequence (rₐ) in a given output library was counted and normalized as follows: (rₐ+1)/total reads for all CRISPR array library elements.

Fold depletion for each CRISPR array was defined as the normalized input read count divided by the normalized output read count (with 1 added to avoid division by zero). A CRISPR array was considered to be strongly depleted if the fold depletion was greater than 3. When calculating the CRISPR array fold-depletion for Cas12i2 across biological replicates, the minimum fold depletion value for a given CRISPR array across all experiments (i.e., a strongly depleted CRISPR array must be strongly depleted in all biological replicates) was taken.

The Protospacer Adjacent Motif (PAM) was then computationally predicted using PAM depletion ratio analysis. A protospacer adjacent sequence was defined as the 4-base pair sequence directly flanking the target (on pACYC184 or E. coli essential genes) on either the 5′ or 3′ end. The following analysis was performed separately for 5′ or 3′ flanking sequences of strongly depleted target sequences (fold depletion greater than 3) to determine the orientation of the PAM. The library count of a protospacer adjacent sequence was defined as the number of instances of the protospacer adjacent sequence in the full CRISPR array library. The depletion count of the protospacer adjacent sequence was defined as the number of instances of the protospacer adjacent sequence from the CRISPR arrays that were strongly depleted. The PAM depletion ratio was defined as the ratio of the depletion count to the library count of a given protospacer adjacent sequence, as follows: PAM Depletion Ratio=(instances of depletion/prevalence in target space). The PAM depletion ratio was calculated for all protospacer adjacent sequences and ranked in descending order, and the highest PAM depletion ratio(s) indicated the PAM specificity.

Table 4 shows potential four nucleotide PAM sequences having a PAM depletion ratio of about 0.5 or higher. These PAM depletion ratios indicate that about 50-70% of the target sequences flanking the sequences in Table 4 were found to be strongly depleted in the E. coli screen.

TABLE 4
PAM sequences and corresponding PAM depletion ratios.

| PAM | PAM Depletion Ratio |
|---|---|
| CTTT | 0.707 |
| CTTC | 0.688 |
| GTTT | 0.612 |
| GTTC | 0.588 |
| TTTC | 0.533 |
| GTTA | 0.513 |
| GTTG | 0.467 |

This Example indicates that several four nucleotide PAMs were identified in E. coli, which can be used in other gene editing applications with Cas12i2.

### Example 2—Determination of PAM Sequences for Cas12i2 System in a Lentiviral System

In this Example, Cas12i2 PAM sequences were determined in a lentiviral screen targeting mammalian genes in 293T cells.

### Native Library Construction

Libraries were first designed for Cas12i2 to target three genes (6 target loci per gene for a total of 18 loci) in order to assess editing efficiency using a self-targeting lentiviral plasmid design. The target sequences are shown in Table 5, with the PAM sequences shown in bold. Libraries were generated by PCR amplification off a U6 promoter geneblock. Each forward primer encoded an RNA guide (a direct repeat and a spacer). The direct repeat sequence used was: AGAAAUCCGUCUUUCAUUGACGG (SEQ ID NO: 17). Each reverse primer encoded the corresponding target sequence flanked by native sequences from the genome (shown in Table 5), a barcode, and an Illumina handle. PCR products encoding the native libraries for the 18 target loci (RNA guide/target pairs) were pooled together. The pooled PCR products were then cloned by Golden Gate cloning into a lentiviral plasmid encoding EF-1 alpha short (EFS)-driven Cas12i2 and IRES-driven puromycin resistance.

TABLE 5
Native Library Target and Flanking Sequences.

| Target | Native 5′ Sequence | Target Sequence | Native 3′ Sequence |
|---|---|---|---|
| AAVS1_1 | GGAAAACTCCCTTTG | TGAGAATGGTGCGTCCTAGG | TGTTCACCAGGTCGT |
| | (SEQ ID NO: 21) | (SEQ ID NO: 22) | (SEQ ID NO: 23) |
| AAVS1_2 | CCTCTTCCGATGTTG | AGCCCCTCCAGCCGGTCCTG | GACTTTGTCTCCTTC |
| | (SEQ ID NO: 24) | (SEQ ID NO: 25) | (SEQ ID NO: 26) |
| AAVS1_3 | TCTGTCTGCAGCTTG | TGGCCTGGGTCACCTCTACG | GCTGGCCCAGATCCT |
| | (SEQ ID NO: 27) | (SEQ ID NO: 28) | (SEQ ID NO: 29) |
| AAVS1_4 | ATACCGTCGGCGTTG | GTGGAGTCCAGCACGGCGCG | GGCGGGCGGCGGCGC |
| | (SEQ ID NO: 30) | (SEQ ID NO: 31) | (SEQ ID NO: 32) |
| AAVS1_5 | GACGGCATGGGGTTG | GGTGAGGGAGGAGAGATGCC | CGGAGAGGACCCAGA |
| | (SEQ ID NO: 33) | (SEQ ID NO: 34) | (SEQ ID NO: 35) |
| AAVS1_6 | TAGGCAGATTCCTTA | TCTGGTGACACACCCCCATT | TCCTGGAGCCATCTC |
| | (SEQ ID NO: 36) | (SEQ ID NO: 37) | (SEQ ID NO: 38) |
| EMX1_1 | GCCGAGGCGGCCTTC | GTGAGTGGCTTCCCTGCCGC | GGCCGCCGCGGGCGC |
| | (SEQ ID NO: 39) | (SEQ ID NO: 40) | (SEQ ID NO: 41) |
| EMX1_2 | GCATATACCAGTTTG | TGGATAAAACTTCTCGGAGG | GTTACTCAGATCAGT |
| | (SEQ ID NO: 42) | (SEQ ID NO: 43) | (SEQ ID NO: 44) |
| EMX1_3 | CCTGACTGTTCCTTG | TGTGACCTGTTCCCACATCT | GGATGGGCTGCAGGA |
| | (SEQ ID NO: 45) | (SEQ ID NO: 46) | (SEQ ID NO: 47) |
| EMX1_4 | CCAAGCCAGAGATTG | TGTGAGGGCCTAGTGGGGTG | TTCATTGAGACAGGC |
| | (SEQ ID NO: 48) | (SEQ ID NO: 49) | (SEQ ID NO: 50) |
| EMX1_5 | TTTCTTAACGTATTG | AGAGGTGGGAATCAGGCCCA | GGTAGTTCAATGGGA |
| | (SEQ ID NO: 51) | (SEQ ID NO: 52) | (SEQ ID NO: 53) |
| EMX1_6 | ACGGCTGGCGTGTTC | TCTTGAGATGGGCTCGGGCT | ACTTGGCCAGCTTCA |
| | (SEQ ID NO: 54) | (SEQ ID NO: 55) | (SEQ ID NO: 56) |
| VEGFA_1 | GGGGGAGGGTGGTTG | GTGCCCTTCGGTCCTCGGCA | CCCCCCTCCGTCTCC |
| | (SEQ ID NO: 57) | (SEQ ID NO: 58) | (SEQ ID NO: 59) |
| VEGFA_2 | GTGGTGGGCATATTC | TGTGCCCGTGGGGACCCCCG | GTTGTGTCCTGTTCG |
| | (SEQ ID NO: 60) | (SEQ ID NO: 61) | (SEQ ID NO: 62) |
| VEGFA_3 | GGAGCGTGTACGTTG | GTGCCCGCTGCTGTCTAATG | CCCTGGAGCCTCCCT |
| | (SEQ ID NO: 63) | (SEQ ID NO: 64) | (SEQ ID NO: 65) |
| VEGFA_4 | AAAGTTCATGGTTTC | GGAGGCCCGACCGGGGCCGG | CGCGGCTCGCGCTCC |
| | (SEQ ID NO: 66) | (SEQ ID NO: 67) | (SEQ ID NO: 68) |
| VEGFA_5 | ATTTTCAGAGGCTTG | TGAGTGCTCCGTGTTAAGGG | GCAGGTAGGATGGGG |
| | (SEQ ID NO: 69) | (SEQ ID NO: 70) | (SEQ ID NO: 71) |
| VEGFA_6 | CAAACAAATGCTTTC | TCCGCTCTGAGCAAGGCCCA | CAGGGACTGCAAAAA |
| | (SEQ ID NO: 72) | (SEQ ID NO: 73) | (SEQ ID NO: 74) |

### PAM Library Construction

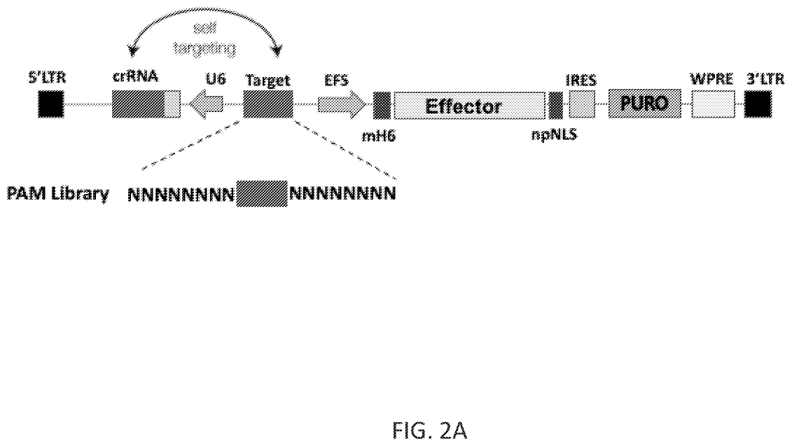

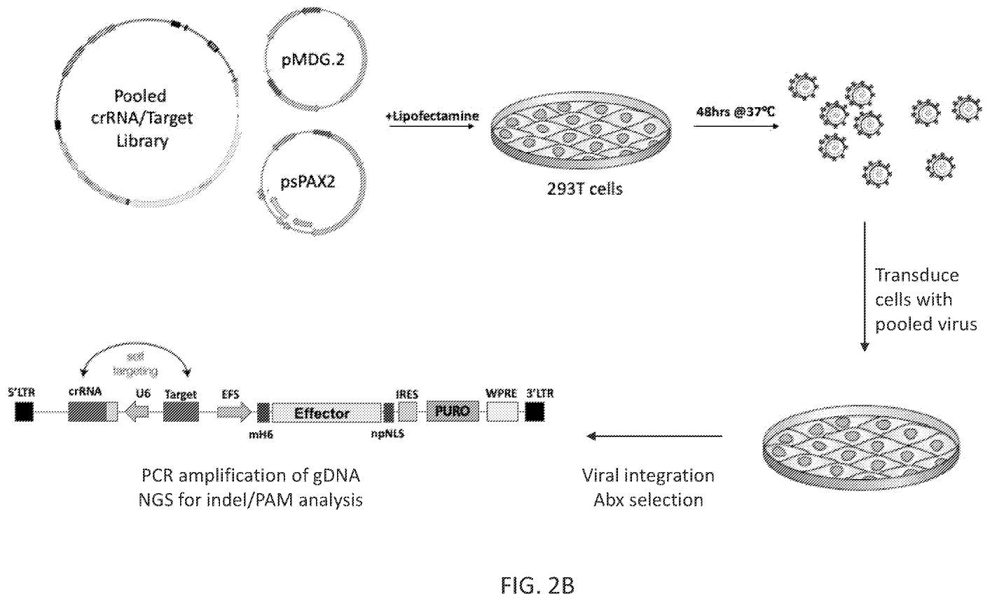

Libraries were next designed to determine optimal PAM sequences for Cas12i2. Libraries were generated by PCR amplification using the forward and reverse primer designs described above for the native libraries. Target sequences from the native library were flanked on the 5′ and 3′ sides by 8 random nucleotides, as shown in Table 6, which were introduced during primer synthesis in order to generate a non-biased library for PAM determination. PCR products corresponding to each target locus of a particular gene target were pooled together, resulting in 3 separate PAM libraries. Pooled PCR products were then cloned into the Cas 1212 plasmid described above. The self-targeting design of the PAM library is shown in FIG. 2A.

TABLE 6
PAM Library Target Sequences.

| Target | 5′ Random Sequence | Target Sequence | 3′ Random Sequence |
|---|---|---|---|
| AAVS1_1 | NNNNNNNN | TGAGAATGGTGCGTCCTAGG | NNNNNNNN |
| | (SEQ ID NO: 75) | (SEQ ID NO: 76) | (SEQ ID NO: 75) |
| AAVS1_2 | NNNNNNNN | AGCCCCTCCAGCCGGTCCTG | NNNNNNNN |
| | (SEQ ID NO: 75) | (SEQ ID NO: 77) | (SEQ ID NO: 75) |
| AAVS1_3 | NNNNNNNN | TGGCCTGGGTCACCTCTACG | NNNNNNNN |
| | (SEQ ID NO: 75) | (SEQ ID NO: 78) | (SEQ ID NO: 75) |
| AAVS1_4 | NNNNNNNN | GTGGAGTCCAGCACGGCGCG | NNNNNNNN |
| | (SEQ ID NO: 75) | (SEQ ID NO: 79) | (SEQ ID NO: 75) |
| AAVS1_5 | NNNNNNNN | GGTGAGGGAGGAGAGATGCC | NNNNNNNN |
| | (SEQ ID NO: 75) | (SEQ ID NO: 80) | (SEQ ID NO: 75) |
| AAVS1_6 | NNNNNNNN | TCTGGTGACACACCCCCATT | NNNNNNNN |
| | (SEQ ID NO: 75) | (SEQ ID NO: 81) | (SEQ ID NO: 75) |
| EMX1_1 | NNNNNNNN | GTGAGTGGCTTCCCTGCCGC | NNNNNNNN |
| | (SEQ ID NO: 75) | (SEQ ID NO: 82) | (SEQ ID NO: 75) |
| EMX1_2 | NNNNNNNN | TGGATAAAACTTCTCGGAGG | NNNNNNNN |
| | (SEQ ID NO: 75) | (SEQ ID NO: 83) | (SEQ ID NO: 75) |
| EMX1_3 | NNNNNNNN | TGTGACCTGTTCCCACATCT | NNNNNNNN |
| | (SEQ ID NO: 75) | (SEQ ID NO: 84) | (SEQ ID NO: 75) |
| EMX1_4 | NNNNNNNN | TGTGAGGGCCTAGTGGGGTG | NNNNNNNN |
| | (SEQ ID NO: 75) | (SEQ ID NO: 85) | (SEQ ID NO: 75) |
| EMX1_5 | NNNNNNNN | AGAGGTGGGAATCAGGCCCA | NNNNNNNN |
| | (SEQ ID NO: 75) | (SEQ ID NO: 86) | (SEQ ID NO: 75) |
| EMX1_6 | NNNNNNNN | TCTTGAGATGGGCTCGGGCT | NNNNNNNN |
| | (SEQ ID NO: 75) | (SEQ ID NO: 87) | (SEQ ID NO: 75) |
| VEGFA_1 | NNNNNNNN | GTGCCCTTCGGTCCTCGGCA | NNNNNNNN |
| | (SEQ ID NO: 75) | (SEQ ID NO: 88) | (SEQ ID NO: 75) |
| VEGFA_2 | NNNNNNNN | TGTGCCCGTGGGGACCCCCG | NNNNNNNN |
| | (SEQ ID NO: 75) | (SEQ ID NO: 89) | (SEQ ID NO: 75) |
| VEGFA_3 | NNNNNNNN | GTGCCCGCTGCTGTCTAATG | NNNNNNNN |
| | (SEQ ID NO: 75) | (SEQ ID NO: 90) | (SEQ ID NO: 75) |
| VEGFA_4 | NNNNNNNN | GGAGGCCCGACCGGGGCCGG | NNNNNNNN |
| | (SEQ ID NO: 75) | (SEQ ID NO: 91) | (SEQ ID NO: 75) |
| VEGFA_5 | NNNNNNNN | TGAGTGCTCCGTGTTAAGGG | NNNNNNNN |
| | (SEQ ID NO: 75) | (SEQ ID NO: 92) | (SEQ ID NO: 75) |
| VEGFA_6 | NNNNNNNN | TCCGCTCTGAGCAAGGCCCA | NNNNNNNN |
| | (SEQ ID NO: 75) | (SEQ ID NO: 93) | (SEQ ID NO: 75) |

### Library Amplification and Lentiviral Packaging

Pooled library plasmids were transformed into electrocompetent cells (Lucigen), plating out approximately 1.5×106 colonies on LB-carbenicillin bioassay plates, which were then scraped and collected in L Broth. DNA was then extracted (Promega Pureyield Midiprep) from pelleted bacteria for each pool. The pooled plasmids were packaged into virus by transfecting 4×106 293T cells with pooled plasmids (6.7 1.1 g) along with the packaging plasmids psPAX2 (3 μg) and pMDG.2 (0.3 μg) using 30 μL Lipofectamine 2000 (Invitrogen) and incubating in DMEM culture medium in 10 cm dishes. After 48 hours of transfection, supernatants were then collected, syringe filtered through a 0.45 PVDF micron filter and stored at −80° C. Viral titer was determined by spinfecting 2.5×105 293T cells with dilutions of virus in the presence of 10 μg/mL of polybrene. After 24 hours of lentivirus treatment, cells were then trypsinized and replated in duplicate at a lower density with or without the addition of 0.5 μg/mL puromycin to the culture media. After selection, cells were counted, and the percentage of transduction was calculated as the cell number from the Puro selected divided by unselected cells. See FIG. 2B.

### Lentiviral Screen

Approximately 1.8×107 293T cells were transduced by spinfection in the presence of 10 μg/mL of polybrene with the viral packaged library pools at MOI=0.15. Transduced cells were then enriched by selection with 0.5 μg/mL of puromycin over several passages. Cells were collected at 5 and 8 days post-transduction. Plasmid DNA pools were used as a negative control. gDNA was harvested from cell pellets using the Zymo Quick-DNA Midiprep Plus kit. NGS primers flanking the designed libraries were used to PCR amplify the gRNA and target sites from gDNA. To ensure >1000X coverage over the library, 20 of gDNA per sample divided up over 8 separate 100 μL PCR reactions were used. Plasmid libraries were also PCR amplified for NGS to serve as background controls. Samples were pooled together, gel extracted, and paired-end sequenced with a 300-cycle NextSeq 500/550 High Output Kit v2 (Illumina). Sequences were then demultiplexed, and indels at each locus were determined. See FIG. 2B.

To calculate editing efficiency in the native libraries, percent indels were calculated by the number of reads with indels divided by the total number of reads for each respective target. Data was background corrected by subtracting out the percent indels calculated for the plasmid controls for each target. Up to 9% editing was observed at various targets from the average of two bioreps, indicating that the system was active and able to be used to identify PAM sequences using the PAM libraries.

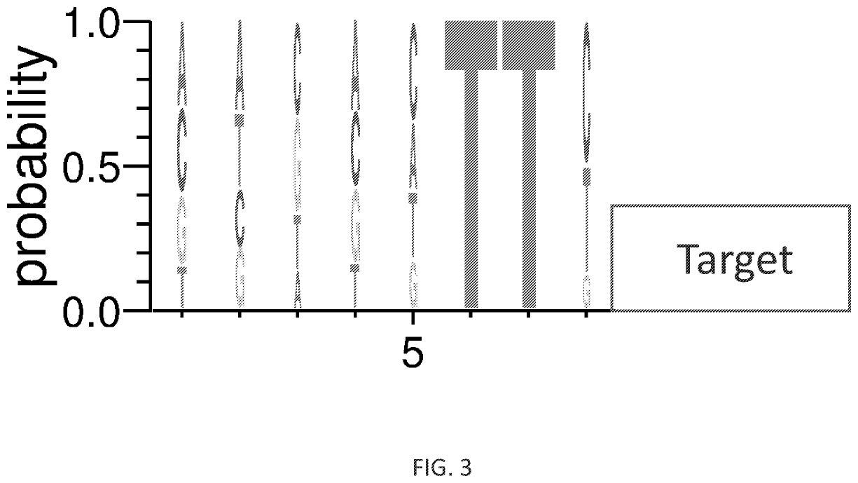

Editing efficiency in PAM library experiments was analyzed as described for the native libraries. For PAM analysis, probability scores were determined for each nucleotide combination 5′ to the target sequence resulting in editing of a target sequence. For example, a nucleotide combination resulting in higher percent indels in one or more targets was ranked higher than a nucleotide combination resulting in lower percent indels in one or more targets. From this analysis, the nucleotide combinations associated with the most efficiently edited target sequences were aligned. In particular, the nucleotide combinations associated with the 10% most edited targets were aligned (e.g., the most edited targets were characterized by higher indel percentages than 90% of the analyzed targets). A Weblogo was generated by uploading the top 10% PAM nucleotide sequence combinations from all combined targets to Weblogo 3.1 (http://weblogo.threeplusone.com/) and is shown in FIG. 3.

In this analysis, enrichment of a base was indicated by font size (e.g., an enriched base was visualized larger than a non-enriched base). Analysis from two bioreplicate experiments revealed an enrichment for an NTTY (e.g., NTTC, NTTT) PAM or an NTTB (e.g., NTTC, NTTG, NTTT) PAM. This includes a CTTY (e.g., CTTC, CTTT) PAM.

This Example indicates that the PAM sequences identified in the mammalian lentiviral screen can be used in additional gene editing applications, such as editing of mammalian targets by transient transfection.

### Example 3—Targeting of Mammalian Genes by Cas12i2 using 5′-NTTN-3′ and 5′-TTN-3′ PAMs

This Example describes targeting of mammalian genes by transient transfection of Cas12i2 using PAM sequences identified in Examples 1 and 2.

To design RNA guides, PAM sequences that were included or were within 20 base pairs of an exon of the coding sequence of AAVS1, EMX1, or VEGFA were identified. Specifically, CTTT and DTTR were used as PAM sequences, all of which were 5′-upstream of the target sequences. TTN was used as a control PAM sequence. The crRNA sequences included the constant region direct repeat (AGAAAUCCGUCUUUCAUUGACGG; SEQ ID NO: 17) upstream of the 20-nucleotide variable region, which consisted of the 20 nucleotides immediately downstream of the PAM.

Wild type Cas12i2 and a Cas12i2 variant of SEQ ID NO: 3 were individually cloned into a pcda3.1 backbone (Invitrogen). The plasmids were then maxi-prepped and diluted to 1 μg/μL. For RNA guide preparation, a dsDNA fragment encoding a crRNA was derived by ultramers containing the target sequence scaffold, and the U6 promoter. Ultramers were resuspended in 10 mM TrisHC1 at a pH of 7.5 to a final stock concentration of 100 μM. Working stocks were subsequently diluted to 10 μM, again using 10 mM TrisHC1 to serve as the template for the PCR reaction. The amplification of the crRNA was done in 50 μL reactions with the following components: 0.02 μl of aforementioned template, 2.5 μl forward primer, 2.5 μl reverse primer, 25 μL NEB HiFi Polymerase, and 20 μl water. Cycling conditions were: 1×(30 s at 98° C.), 30×(10 s at 98° C., 15 s at 67° C.), 1×(2 min at 72° C.). PCR products were cleaned up with a 1.8X SPRI treatment and normalized to 25 ng/μL with nuclease-free dH₂O.

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine 2000 and 9.5 μL of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of crRNA and water up to 10 μL (Solution 2). The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells.

72 hours post transfection, cells were trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes. Samples were kept frozen at −20° C. or used immediately for PCR.

Samples for Next Generation Sequencing were prepared by two rounds of PCR. The first round (PCR1) was used to amplify specific genomic regions depending on the target. PCR products were run on 2% E-Gel (EtBr) to check for proper size (˜250 bp). Round 2 PCR (PCR2) was done to add Illumina adapters and indexes. Reactions were then pooled (2 μL each), loaded onto a 2% E-Gel EX to check for size, and then column purified. Sequencing runs (paired end) were performed with a 150 cycle NextSeq v2.5 mid or high output kit.

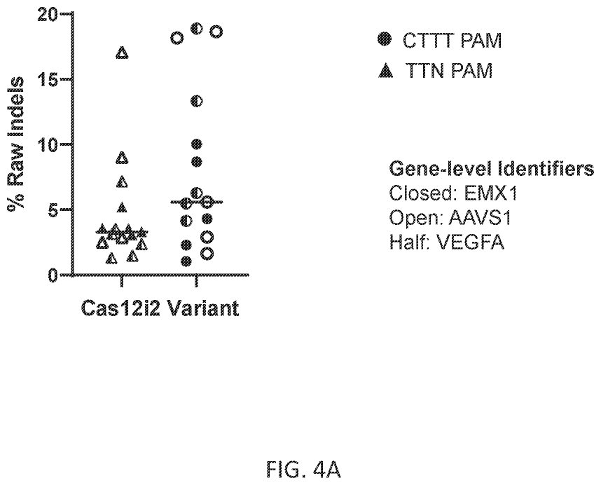

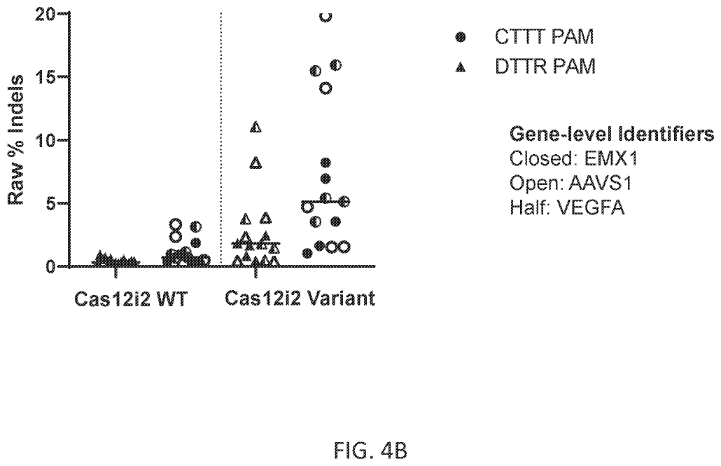

Indel rates at each target were determined and compared between PAM conditions. Edited targets were defined as targets that showed indel levels above background (>0.5% in this assay). FIG. 4A shows percent indels measured for a variant Cas12i2 (SEQ ID NO: 3) using the CTTT PAM (circles) and TTN PAM (triangles). FIG. 4B shows percent indels measured for wild-type Cas12i2 and a variant Cas12i2 (SEQ ID NO: 3) using the CTTT PAM (circles) and DTTR PAM (triangles). In FIG. 4A and FIG. 4B, open circles/triangles represent AAVS1 targets, closed circles/triangles represent EMX1 targets, and half shaded circles/triangles represent VEGFA targets. The bars represented median indels across all targets tested. As shown in FIG. 4A and FIG. 4B, use of a CTTT PAM results in the highest median indel levels in each of the three genes (AAVS1, EMX1, and VEGFA).

Additional PAM sequences identified in Examples 1 and 2, such as CTTC, GTTT, and GTTC, can be assessed using this described method. Therefore, this Example shows that indel activity can be measured at multiple mammalian target loci having various PAM sequences.

### Example 4—Targeting of Mammalian Genes with Cas12i2 and Modified RNA Guide Sequences

This Example describes targeting of mammalian genes by the variant Cas12i2 of SEQ ID NO: 4 using RNA guides having spacers that are substantially complementary to the target sequences. The spacers of the RNA guide had either one mismatch between the spacer sequence and target sequence or two consecutive mismatches between the spacer sequence and the target sequence. Cas12i2 plasmids and RNA guides were prepared as described in Example 3. The prepared crRNA sequences and their corresponding target sequences are shown in Table 7. The PAM sequence was 5′-CTTT-3′ for each target. Cells were transfected and harvested, and samples were prepared for Next Generation sequencing as described in Example 3.

TABLE 7
Target and crRNA sequences.

| Target | RNA Guide Sequence | Mismatch |
|---|---|---|
| GTAGCCTCTCCCGCTC | AGAAATCCGTCTTTCATTGACGGGTAGC | No mismatch |
| TGGT | CTCTCCCGCTCTGGT (SEQ ID NO: 96) | |
| (AAVS1; SEQ ID NO: 94) | | |
| AAVS1 (SEQ ID NO: 94) | AGAAAUCCGUCUUUCAUUGACGGCUAG | Single (Position 1) |
| | CCUCUCCCGCUCUGGU (SEQ ID NO: 97) | |
| AAVS1 (SEQ ID NO: 94) | AGAAAUCCGUCUUUCAUUGACGGGAAG | Single (Position 2) |
| | CCUCUCCCGCUCUGGU (SEQ ID NO: 98) | |
| AAVS1 (SEQ ID NO: 94) | AGAAAUCCGUCUUUCAUUGACGGGUUG | Single (Position 3) |
| | CCUCUCCCGCUCUGGU (SEQ ID NO: 99) | |
| AAVS1 (SEQ ID NO: 94) | AGAAAUCCGUCUUUCAUUGACGGGUAC | Single (Position 4) |
| | CCUCUCCCGCUCUGGU (SEQ ID NO: 100) | |
| AAVS1 (SEQ ID NO: 94) | AGAAAUCCGUCUUUCAUUGACGGCUAG | Single (Position 5) |
| | CCUCUCCCGCUCUGGU (SEQ ID NO: 101) | |
| AAVS1 (SEQ ID NO: 94) | AGAAAUCCGUCUUUCAUUGACGGCUAG | Single (Position 6) |
| | CCUCUCCCGCUCUGGU (SEQ ID NO: 102) | |
| AAVS1 (SEQ ID NO: 94) | AGAAAUCCGUCUUUCAUUGACGGCUAG | Single (Position 7) |
| | CCACUCCCGCUCUGGU (SEQ ID NO: 103) | |
| AAVS1 (SEQ ID NO: 94) | AGAAAUCCGUCUUUCAUUGACGGGUAG | Single (Position 8) |
| | CCUGUCCCGCUCUGGU (SEQ ID NO: 104) | |
| AAVS1 (SEQ ID NO: 94) | AGAAAUCCGUCUUUCAUUGACGGGUAG | Single (Position 9) |
| | CCUCACCCGCUCUGGU (SEQ ID NO: 105) | |
| AAVS1 (SEQ ID NO: 94) | AGAAAUCCGUCUUUCAUUGACGGGUAG | Single (Position 10) |
| | CCUCUGCCGCUCUGGU (SEQ ID NO: 106) | |
| AAVS1 (SEQ ID NO: 94) | AGAAAUCCGUCUUUCAUUGACGGGUAG | Single (Position 11) |
| | CCUCUCGCGCUCUGGU (SEQ ID NO: 107) | |
| AAVS1 (SEQ ID NO: 94) | AGAAAUCCGUCUUUCAUUGACGGGUAG | Single (Position 12) |
| | CCUCUCCGGCUCUGGU (SEQ ID NO: 108) | |
| AAVS1 (SEQ ID NO: 94) | AGAAAUCCGUCUUUCAUUGACGGGUAG | Single (Position 13) |
| | CCUCUCCCCCUCUGGU (SEQ ID NO: 109) | |
| AAVS1 (SEQ ID NO: 94) | AGAAAUCCGUCUUUCAUUGACGGGUAG | Single (Position 14) |
| | CCUCUCCCGCUCUGGU (SEQ ID NO: 110) | |
| AAVS1 (SEQ ID NO: 94) | AGAAAUCCGUCUUUCAUUGACGGGUAG | Single (Position 15) |
| | CCUCUCCCGCACUGGU (SEQ ID NO: 111) | |
| AAVS1 (SEQ ID NO: 94) | AGAAAUCCGUCUUUCAUUGACGGGUAG | Single (Position 16) |
| | CCUCUCCCGCUGUGGU (SEQ ID NO: 112) | |
| AAVS1 (SEQ ID NO: 94) | AGAAAUCCGUCUUUCAUUGACGGGUAG | Single (Position 17) |
| | CCUCUCCCGCUCAGGU (SEQ ID NO: 113) | |
| AAVS1 (SEQ ID NO: 94) | AGAAAUCCGUCUUUCAUUGACGGGUAG | Single (Position 18) |
| | CCUCUCCCGCUCUCGU (SEQ ID NO: 114) | |
| AAVS1 (SEQ ID NO: 94) | AGAAAUCCGUCUUUCAUUGACGGGUAG | Single (Position 19) |
| | CCUCUCCCGCUCUGGU (SEQ ID NO: 115) | |
| AAVS1 (SEQ ID NO: 94) | AGAAAUCCGUCUUUCAUUGACGGGUAG | Single (Position 20) |
| | CCUCUCCCGCUCUGGA (SEQ ID NO: 116) | |
| AAVS1 (SEQ ID NO: 94) | AGAAAUCCGUCUUUCAUUGACGGCAAG | Double (Positions 1 and |
| | CCUCUCCCGCUCUGGU (SEQ ID NO: 117) | 2) |
| AAVS1 (SEQ ID NO: 94) | AGAAAUCCGUCUUUCAUUGACGGGAUG | Double (Positions 2 and |
| | CCUCUCCCGCUCUGGU (SEQ ID NO: 118) | 3) |
| AAVS1 (SEQ ID NO: 94) | AGAAAUCCGUCUUUCAUUGACGGGUUC | Double (Positions 3 and |
| | CCUCUCCCGCUCUGGU (SEQ ID NO: 119) | 4) |
| AAVS1 (SEQ ID NO: 94) | AGAAAUCCGUCUUUCAUUGACGGGUAG | Double (Positions 4 and |
| | CCUCUCCCGCUCUGGU (SEQ ID NO: 120) | 5) |
| AAVS1 (SEQ ID NO: 94) | AGAAAUCCGUCUUUCAUUGACGGGUAG | Double (Positions 5 and |
| | GGUCUCCCGCUCUGGU (SEQ ID NO: 121) | 6) |
| AAVS1 (SEQ ID NO: 94) | AGAAAUCCGUCUUUCAUUGACGGGUAG | Double (Positions 6 and |
| | CGACUCCCGCUCUGGU (SEQ ID NO: 122) | 7) |
| AAVS1 (SEQ ID NO: 94) | AGAAAUCCGUCUUUCAUUGACGGGUAG | Double (Positions 7 and |
| | CCAGUCCCGCUCUGGU (SEQ ID NO: 123) | 8) |
| AAVS1 (SEQ ID NO: 94) | AGAAAUCCGUCUUUCAUUGACGGGUAG | Double (Positions 8 and |
| | CCUGACCCGCUCUGGU (SEQ ID NO: 124) | 9) |
| AAVS1 (SEQ ID NO: 94) | AGAAAUCCGUCUUUCAUUGACGGGUAG | Double (Positions 9 and |
| | CCUCAGCCGCUCUGGU (SEQ ID NO: 125) | 10) |
| AAVS1 (SEQ ID NO: 94) | AGAAAUCCGUCUUUCAUUGACGGGUAG | Double (Positions 10 |
| | CCUCUGGCGCUCUGGU (SEQ ID NO: 126) | and 11) |
| AAVS1 (SEQ ID NO: 94) | AGAAAUCCGUCUUUCAUUGACGGGUAG | Double (Positions 11 |
| | CCUCUCGGGCUCUGGU (SEQ ID NO: 127) | and 12) |
| AAVS1 (SEQ ID NO: 94) | AGAAAUCCGUCUUUCAUUGACGGGUAG | Double (Positions 12 |
| | CCUCUCCGCCUCUGGU (SEQ ID NO: 128) | and 13) |
| AAVS1 (SEQ ID NO: 94) | AGAAAUCCGUCUUUCAUUGACGGGUAG | Double (Positions 13 |
| | CCUCUCCCCGUCUGGU (SEQ ID NO: 129) | and 14) |
| AAVS1 (SEQ ID NO: 94) | AGAAAUCCGUCUUUCAUUGACGGGUAG | Double (Positions 14 |
| | CCUCUCCCGGACUGGU (SEQ ID NO: 130) | and 15) |
| AAVS1 (SEQ ID NO: 94) | AGAAAUCCGUCUUUCAUUGACGGGUAG | Double (Positions 15 |
| | CCUCUCCCGCAGUGGU (SEO ID NO: 131) | and 16) |
| AAVS1 (SEQ ID NO: 94) | AGAAAUCCGUCUUUCAUUGACGGGUAG | Double (Positions 16 |
| | CCUCUCCCGCUGAGGU (SEQ ID NO: 132) | and 17) |
| AAVS1 (SEQ ID NO: 94) | AGAAAUCCGUCUUUCAUUGACGGGUAG | Double (Positions 17 |
| | CCUCUCCCGCUCACGU (SEQ ID NO: 133) | and 18) |
| AAVS1 (SEQ ID NO: 94) | AGAAAUCCGUCUUUCAUUGACGGGUAG | Double (Positions 18 |
| | CCUCUCCCGCUCUCCU (SEQ ID NO: 134) | and 19) |
| AAVS1 (SEQ ID NO: 94) | AGAAAUCCGUCUUUCAUUGACGGGUAG | Double (Positions 19 |
| | CCUCUCCCGCUCUGCA (SEQ ID NO: 135) | and 20) |
| GGGGAGGCCTGGAGT | AGAAAUCCGUCUUUCAUUGACGGGGGG | No mismatch |
| CATGG | AGGCCUGGAGUCAUGG (SEQ ID NO: 136) | |
| (EMX1; SEQ ID NO: 95) | | |
| EMX1 (SEQ ID NO: 95) | AGAAAUCCGUCUUUCAUUGACGGGGGG | Single (Position 1) |
| | AGGCCUGGAGUCAUGG (SEQ ID NO: 137) | |
| EMX1 (SEQ ID NO: 95) | AGAAAUCCGUCUUUCAUUGACGGGGGG | Single (Position 2) |
| | AGGCCUGGAGUCAUGG (SEQ ID NO: 138) | |
| EMX1 (SEQ ID NO: 95) | AGAAAUCCGUCUUUCAUUGACGGGGGG | Single (Position 3) |
| | AGGCCUGGAGUCAUGG (SEQ ID NO: 139) | |
| EMX1 (SEQ ID NO: 95) | AGAAAUCCGUCUUUCAUUGACGGGGGG | Single (Position 4) |
| | AGGCCUGGAGUCAUGG (SEQ ID NO: 140) | |
| EMX1 (SEQ ID NO: 95) | AGAAAUCCGUCUUUCAUUGACGGGGGG | Single (Position 5) |
| | UGGCCUGGAGUCAUGG (SEQ ID NO: 141) | |
| EMX1 (SEQ ID NO: 95) | AGAAAUCCGUCUUUCAUUGACGGGGGG | Single (Position 6) |
| | AGGCCUGGAGUCAUGG (SEQ ID NO: 142) | |
| EMX1 (SEQ ID NO: 95) | AGAAAUCCGUCUUUCAUUGACGGGGGG | Single (Position 7) |
| | AGGCCUGGAGUCAUGG (SEQ ID NO: 143) | |
| EMX1 (SEQ ID NO: 95) | AGAAAUCCGUCUUUCAUUGACGGGGGG | Single (Position 8) |
| | AGGCCUGGAGUCAUGG (SEQ ID NO: 144) | |
| EMX1 (SEQ ID NO: 95) | AGAAAUCCGUCUUUCAUUGACGGGGGG | Single (Position 9) |
| | AGGCCUGGAGUCAUGG (SEQ ID NO: 145) | |
| EMX1 (SEQ ID NO: 95) | AGAAAUCCGUCUUUCAUUGACGGGGGG | Single (Position 10) |
| | AGGCCAGGAGUCAUGG (SEQ ID NO: 146) | |
| EMX1 (SEQ ID NO: 95) | AGAAAUCCGUCUUUCAUUGACGGGGGG | Single (Position 11) |
| | AGGCCUGGAGUCAUGG (SEQ ID NO: 147) | |
| EMX1 (SEQ ID NO: 95) | AGAAAUCCGUCUUUCAUUGACGGGGGG | Single (Position 12) |
| | AGGCCUGGAGUCAUGG (SEQ ID NO: 148) | |
| EMX1 (SEQ ID NO: 95) | AGAAAUCCGUCUUUCAUUGACGGGGGG | Single (Position 13) |
| | AGGCCUGGUGUCAUGG (SEQ ID NO: 149) | |
| EMX1 (SEQ ID NO: 95) | AGAAAUCCGUCUUUCAUUGACGGGGGG | Single (Position 14) |
| | AGGCCUGGAGUCAUGG (SEQ ID NO: 150) | |
| EMX1 (SEQ ID NO: 95) | AGAAAUCCGUCUUUCAUUGACGGGGGG | Single (Position 15) |
| | AGGCCUGGAGACAUGG (SEQ ID NO: 151) | |
| EMX1 (SEQ ID NO: 95) | AGAAAUCCGUCUUUCAUUGACGGGGGG | Single (Position 16) |
| | AGGCCUGGAGUCAUGG (SEQ ID NO: 152) | |
| EMX1 (SEQ ID NO: 95) | AGAAAUCCGUCUUUCAUUGACGGGGGG | Single (Position 17) |
| | AGGCCUGGAGUCUUGG (SEQ ID NO: 153) | |
| EMX1 (SEQ ID NO: 95) | AGAAAUCCGUCUUUCAUUGACGGGGGG | Single (Position 18) |
| | AGGCCUGGAGUCAAGG (SEQ ID NO: 154) | |
| EMX1 (SEQ ID NO: 95) | AGAAAUCCGUCUUUCAUUGACGGGGGG | Single (Position 19) |
| | AGGCCUGGAGUCAUGG (SEQ ID NO: 155) | |
| EMX1 (SEQ ID NO: 95) | AGAAAUCCGUCUUUCAUUGACGGGGGG | Single (Position 20) |
| | AGGCCUGGAGUCAUGG (SEQ ID NO: 156) | |
| EMX1 (SEQ ID NO: 95) | AGAAAUCCGUCUUUCAUUGACGGCCGG | Double (Positions 1 and |
| | AGGCCUGGAGUCAUGG (SEQ ID NO: 157) | 2) |
| EMX1 (SEQ ID NO: 95) | AGAAAUCCGUCUUUCAUUGACGGGCCG | Double (Positions 2 and |
| | AGGCCUGGAGUCAUGG (SEQ ID NO: 158) | 3) |
| EMX1 (SEQ ID NO: 95) | AGAAAUCCGUCUUUCAUUGACGGGGCC | Double (Positions 3 and |
| | AGGCCUGGAGUCAUGG (SEQ ID NO: 159) | 4) |
| EMX1 (SEQ ID NO: 95) | AGAAAUCCGUCUUUCAUUGACGGGGGC | Double (Positions 4 and |
| | UGGCCUGGAGUCAUGG (SEQ ID NO: 160) | 5) |
| EMX1 (SEQ ID NO: 95) | AGAAAUCCGUCUUUCAUUGACGGGGGG | Double (Positions 5 and |
| | UGGCCUGGAGUCAUGG (SEQ ID NO: 161) | 6) |
| EMX1 (SEQ ID NO: 95) | AGAAAUCCGUCUUUCAUUGACGGGGGG | Double (Positions 6 and |
| | ACCCCUGGAGUCAUGG (SEQ ID NO: 162) | 7) |
| EMX1 (SEQ ID NO: 95) | AGAAAUCCGUCUUUCAUUGACGGGGGG | Double (Positions 7 and |
| | AGCGCUGGAGUCAUGG (SEQ ID NO: 163) | 8) |
| EMX1 (SEQ ID NO: 95) | AGAAAUCCGUCUUUCAUUGACGGGGGG | Double (Positions 8 and |
| | AGGGGUGGAGUCAUGG (SEQ ID NO: 164) | 9) |
| EMX1 (SEQ ID NO: 95) | AGAAAUCCGUCUUUCAUUGACGGGGGG | Double (Positions 9 and |
| | AGGCGAGGAGUCAUGG (SEQ ID NO: 165) | 10) |
| EMX1 (SEQ ID NO: 95) | AGAAAUCCGUCUUUCAUUGACGGGGGG | Double (Positions 10 |
| | AGGCCACGAGUCAUGG (SEQ ID NO: 166) | and 11) |
| EMX1 (SEQ ID NO: 95) | AGAAAUCCGUCUUUCAUUGACGGGGGG | Double (Positions 11 |
| | AGGCCUCCAGUCAUGG (SEQ ID NO: 167) | and 12) |
| EMX1 (SEQ ID NO: 95) | AGAAAUCCGUCUUUCAUUGACGGGGGG | Double (Positions 12 |
| | AGGCCUGCUGUCAUGG (SEQ ID NO: 168) | and 13) |
| EMX1 (SEQ ID NO: 95) | AGAAAUCCGUCUUUCAUUGACGGGGGG | Double (Positions 13 |
| | AGGCCUGGUCUCAUGG (SEQ ID NO: 169) | and 14) |
| EMX1 (SEQ ID NO: 95) | AGAAAUCCGUCUUUCAUUGACGGGGGG | Double (Positions 14 |
| | AGGCCUGGACACAUGG (SEQ ID NO: 170) | and 15) |
| EMX1 (SEQ ID NO: 95) | AGAAAUCCGUCUUUCAUUGACGGGGGG | Double (Positions 15 |
| | AGGCCUGGAGAGAUGG (SEQ ID NO: 171) | and 16) |
| EMX1 (SEQ ID NO: 95) | AGAAAUCCGUCUUUCAUUGACGGGGGG | Double (Positions 16 |
| | AGGCCUGGAGUGUUGG (SEQ ID NO: 172) | and 17) |
| EMX1 (SEQ ID NO: 95) | AGAAAUCCGUCUUUCAUUGACGGGGGG | Double (Positions 17 |
| | AGGCCUGGAGUCUAGG (SEQ ID NO: 173) | and 18) |
| EMX1 (SEQ ID NO: 95) | AGAAAUCCGUCUUUCAUUGACGGGGGG | Double (Positions 18 |
| | AGGCCUGGAGUCAACG (SEQ ID NO: 174) | and 19) |
| EMX1 (SEQ ID NO: 95) | AGAAAUCCGUCUUUCAUUGACGGGGGG | Double (Positions 19 |
| | AGGCCUGGAGUCAUCC (SEQ ID NO: 175) | and 20) |

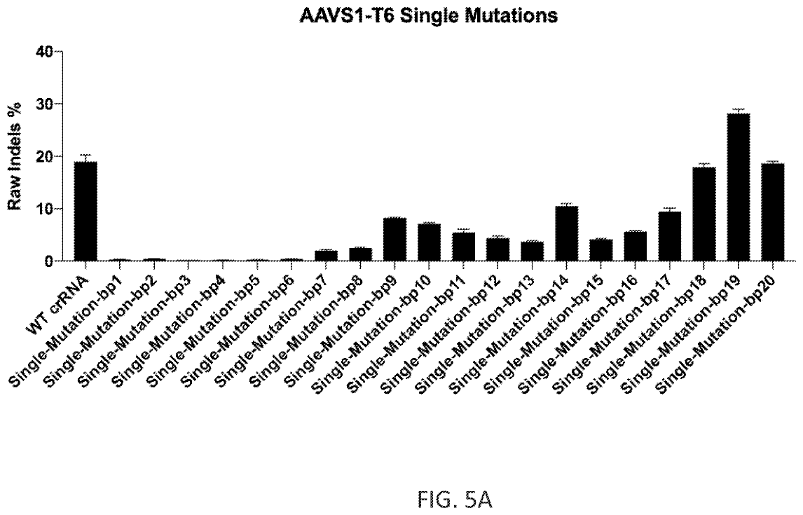

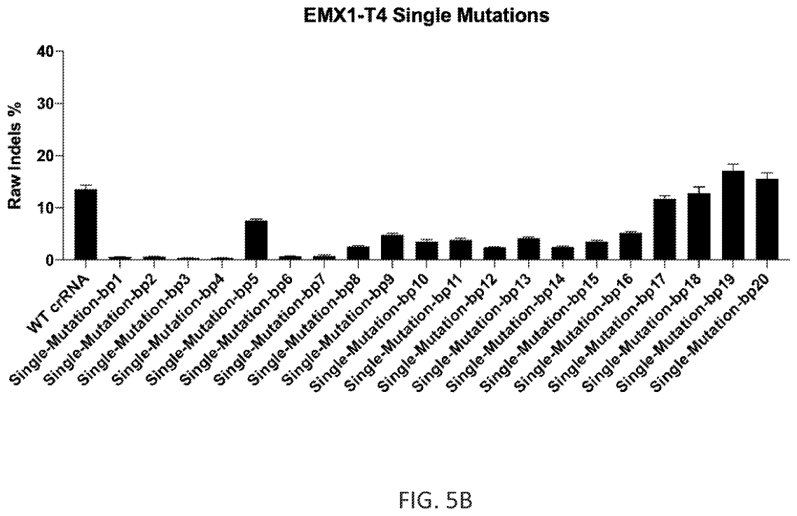

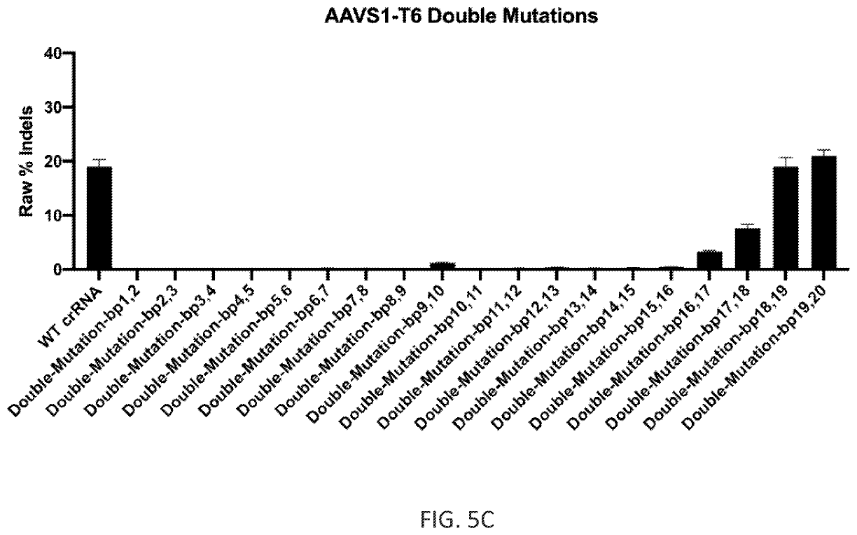

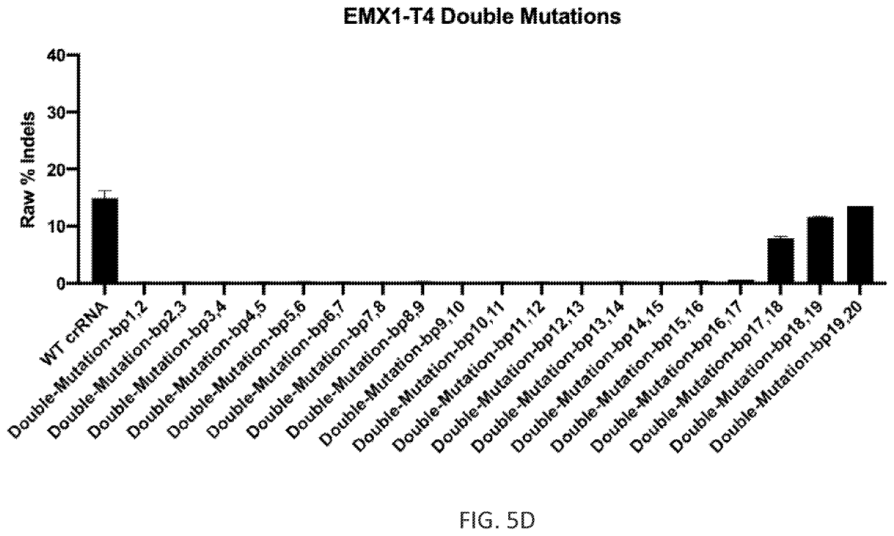

Indel activity using RNA guides having single mismatches between the spacer sequence and either the AAVS1 target sequence or EMX1 target sequence is shown in FIG. 5A and FIG. 5B, respectively. Indel activity using RNA guides having double mismatches between the spacer sequence and either the AAVS1 target sequence or EMX1 target sequence is shown in FIG. 5C and FIG. 5D, respectively. Cas12i2 was tolerant of mismatches, with single mismatches being better tolerated than double mismatches. Both single and double mismatches at the PAM-distal region of the spacer (e.g., at nucleotide positions 17-20) were tolerated by Cas12i2.

Therefore, Cas12i2 exhibits indel activity when the spacer sequence of a Cas12i2 RNA guide is substantially complementary to the target sequence.

What is claimed is:

1. A composition comprising:
(a) an RNA guide or a nucleic acid encoding the RNA guide, wherein the RNA guide comprises a direct repeat sequence and a spacer sequence; and
(b) a Cas12i2 polypeptide or a nucleic acid encoding the Cas12i2 polypeptide, wherein the RNA guide forms a complex with the Cas12i2 polypeptide, and wherein the spacer sequence binds to a target sequence adjacent to a protospacer adjacent motif (PAM) sequence comprising 4 nucleotides.

2. The composition of claim 1, wherein the PAM sequence comprises the sequence 5′-NTTN-3′, wherein N is any nucleotide.

3. The composition of claim 1 or claim 2, wherein the PAM sequence comprises the sequence 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein N is any nucleotide, Y is C or T, B is any nucleotide except for A, D is any nucleotide except for C, and R is A or G.

4. The composition of claim 1 or claim 2, wherein the PAM sequence comprises the sequence 5′-CTTT-3′, 5′-CTTC-3′, 5′-GTTT-3′, 5′-GTTC-3′, 5′-TTTC-3′, 5′-GTTA-3′, or 5′-GTTG-3′.

5. The composition of claim 1 or claim 2, wherein the spacer sequence comprises between 10 and 50 nucleotides in length.

6. The composition of claim 1 or claim 2, wherein the spacer sequence comprises between 15 and 35 nucleotides in length.

7. The composition of claim 1 or claim 2, wherein:
a. nucleotide 1 through nucleotide 5 of the spacer sequence comprise have 100% complementarity to the target nucleic acid;
b. nucleotide 6 through nucleotide 10 of the spacer sequence comprise at least 80% complementarity to the target nucleic acid;
c. nucleotide 11 through nucleotide 15 of the spacer sequence comprise at least 80% complementarity to the target nucleic acid;
d. nucleotide 16 through nucleotide 20 of the spacer sequence comprise at least 60% complementarity to the target nucleic acid;
e. nucleotide 1 through nucleotide 10 of the spacer sequence comprise at least 80% complementarity to the target nucleic acid;
f. nucleotide 1 through nucleotide 15 of the spacer sequence comprise at least 80% complementarity to the target nucleic acid;
g. nucleotide 1 through nucleotide 20 of the spacer sequence comprise at least 80% complementarity to the target nucleic acid;
h. nucleotide 5 through nucleotide 15 of the spacer sequence comprise at least 80% complementarity to the target nucleic acid;
i. nucleotide 5 through nucleotide 20 of the spacer sequence comprise at least 80% complementarity to the target nucleic acid; and/or
j. nucleotide 10 through nucleotide 20 of the spacer sequence comprise at least 60% complementarity to the target nucleic acid.

8. The composition of claim 1 or claim 2, wherein the direct repeat sequence comprises at least 90% identity to SEQ ID NO: 18 or SEQ ID NO: 19.

9. The composition of claim 1 or claim 2, wherein the direct repeat sequence comprises at least 95% identity to SEQ ID NO: 18 or SEQ ID NO: 19.

10. The composition of claim 1 or claim 2, wherein the direct repeat sequence comprises SEQ ID NO:
18 or SEQ NO: 19.

11. The composition of claim 1 or claim 2, wherein the direct repeat sequence comprises:
a. nucleotide 1 through nucleotide 36 of a sequence that is at least 90% identical to a sequence of any one of SEQ ID NOs: 8-15;
b. nucleotide 2 through nucleotide 36 of a sequence that is at least 90% identical to a sequence of any one of SEQ ID NOs: 8-15;
c. nucleotide 3 through nucleotide 36 of a sequence that is at least 90% identical to a sequence of any one of SEQ ID NOs: 8-15;
d. nucleotide 4 through nucleotide 36 of a sequence that is at least 90% identical to a sequence of any one of SEQ ID NOs: 8-15;
e. nucleotide 5 through nucleotide 36 of a sequence that is at least 90% identical to a sequence of any one of SEQ ID NOs: 8-15;
f. nucleotide 6 through nucleotide 36 of a sequence that is at least 90% identical to a sequence of any one of SEQ ID NOs: 8-15;
g. nucleotide 7 through nucleotide 36 of a sequence that is at least 90% identical to a sequence of any one of SEQ ID NOs: 8-15;
h. nucleotide 8 through nucleotide 36 of a sequence that is at least 90% identical to a sequence of any one of SEQ ID NOs: 8-15;
i. nucleotide 9 through nucleotide 36 of a sequence that is at least 90% identical to a sequence of any one of SEQ ID NOs: 8-15;
j. nucleotide 10 through nucleotide 36 of a sequence that is at least 90% identical to a sequence of any one of SEQ ID NOs: 8-15;
k. nucleotide 11 through nucleotide 36 of a sequence that is at least 90% identical to a sequence of any one of SEQ ID NOs: 8-15;
l. nucleotide 12 through nucleotide 36 of a sequence that is at least 90% identical to a sequence of any one of SEQ ID NOs: 8-15;
m. nucleotide 13 through nucleotide 36 of a sequence that is at least 90% identical to a sequence of any one of SEQ ID NOs: 8-15;
n. nucleotide 14 through nucleotide 36 of a sequence that is at least 90% identical to a sequence of any one of SEQ ID NOs: 8-15;
o. nucleotide 1 through nucleotide 34 of a sequence that is at least 90% identical to a sequence of SEQ ID NO: 16;
p. nucleotide 2 through nucleotide 34 of a sequence that is at least 90% identical to a sequence of SEQ ID NO: 16;
q. nucleotide 3 through nucleotide 34 of a sequence that is at least 90% identical to a sequence of SEQ ID NO: 16;
r. nucleotide 4 through nucleotide 34 of a sequence that is at least 90% identical to a sequence of SEQ ID NO: 16;
s. nucleotide 5 through nucleotide 34 of a sequence that is at least 90% identical to a sequence of SEQ ID NO: 16;
t. nucleotide 6 through nucleotide 34 of a sequence that is at least 90% identical to a sequence of SEQ ID NO: 16;
u. nucleotide 7 through nucleotide 34 of a sequence that is at least 90% identical to a sequence of SEQ ID NO: 16;
v. nucleotide 8 through nucleotide 34 of a sequence that is at least 90% identical to a sequence of SEQ ID NO: 16;
w. nucleotide 9 through nucleotide 34 of a sequence that is at least 90% identical to a sequence of SEQ ID NO: 16;
x. nucleotide 10 through nucleotide 34 of a sequence that is at least 90% identical to a sequence of SEQ ID NO: 16;
y. nucleotide 11 through nucleotide 34 of a sequence that is at least 90% identical to a sequence of SEQ ID NO: 16;
z. nucleotide 12 through nucleotide 34 of a sequence that is at least 90% identical to a sequence of SEQ ID NO: 16; or
aa. a sequence that is at least 90% identical to a sequence of SEQ ID NO: 17 or a portion thereof.

12. The composition of claim 1 or claim 2, wherein the direct repeat sequence comprises:
a. nucleotide 1 through nucleotide 36 of any one of SEQ ID NOs: 8-15;
b. nucleotide 2 through nucleotide 36 of any one of SEQ ID NOs: 8-15;
c. nucleotide 3 through nucleotide 36 of any one of SEQ ID NOs: 8-15;
d. nucleotide 4 through nucleotide 36 of any one of SEQ ID NOs: 8-15;
e. nucleotide 5 through nucleotide 36 of any one of SEQ ID NOs: 8-15;
f. nucleotide 6 through nucleotide 36 of any one of SEQ ID NOs: 8-15;
g. nucleotide 7 through nucleotide 36 of any one of SEQ ID NOs: 8-15;
h. nucleotide 8 through nucleotide 36 of any one of SEQ ID NOs: 8-15;
i. nucleotide 9 through nucleotide 36 of any one of SEQ ID NOs: 8-15;
j. nucleotide 10 through nucleotide 36 of any one of SEQ ID NOs: 8-15;
k. nucleotide 11 through nucleotide 36 of any one of SEQ ID NOs: 8-15;
l. nucleotide 12 through nucleotide 36 of any one of SEQ ID NOs: 8-15;
m. nucleotide 13 through nucleotide 36 of any one of SEQ ID NOs: 8-15;
n. nucleotide 14 through nucleotide 36 of any one of SEQ ID NOs: 8-15;
o. nucleotide 1 through nucleotide 34 of SEQ ID NO: 16;
p. nucleotide 2 through nucleotide 34 of SEQ ID NO: 16;
q. nucleotide 3 through nucleotide 34 of SEQ ID NO: 16;
r. nucleotide 4 through nucleotide 34 of SEQ ID NO: 16;
s. nucleotide 5 through nucleotide 34 of SEQ ID NO: 16;
t. nucleotide 6 through nucleotide 34 of SEQ ID NO: 16;
u. nucleotide 7 through nucleotide 34 of SEQ ID NO: 16;
v. nucleotide 8 through nucleotide 34 of SEQ ID NO: 16;
w. nucleotide 9 through nucleotide 34 of SEQ ID NO: 16;
x. nucleotide 10 through nucleotide 34 of SEQ ID NO: 16;
y. nucleotide 11 through nucleotide 34 of SEQ ID NO: 16;
z. nucleotide 12 through nucleotide 34 of SEQ ID NO: 16; or
aa. SEQ ID NO: 17 or a portion thereof.

13. The composition of claim 1 or claim 2, wherein the direct repeat sequence comprises:
a. a first portion having at least 90% identity to nucleotides 1-13 of SEQ ID NO: 17 and a second portion having at least 90% identity to nucleotides 14-23;
b. a first portion having at least 90% identity to nucleotides 1-14 of SEQ ID NO: 17 and a second portion having at least 90% identity to nucleotides 15-23;
c. a first portion having at least 90% identity to nucleotides 1-15 of SEQ ID NO: 17 and a second portion having at least 90% identity to nucleotides 16-23; or
d. a first portion having at least 90% identity to nucleotides 1-16 of SEQ ID NO: 17 and a second portion having at least 90% identity to nucleotides 17-23; and
a heterologous sequence between the first portion and the second portion.

14. The composition of claim 1 or claim 2, wherein the direct repeat sequence comprises:
a. a first portion comprising nucleotides 1-13 of SEQ ID NO: 17 and a second portion comprising nucleotides 14-23;
b. a first portion comprising nucleotides 1-14 of SEQ ID NO: 17 and a second portion comprising nucleotides 15-23;
c. a first portion comprising nucleotides 1-15 of SEQ ID NO: 17 and a second portion comprising nucleotides 16-23; or
d. a first portion comprising nucleotides 1-16 of SEQ ID NO: 17 and a second portion comprising nucleotides 17-23; and
a heterologous sequence between the first portion and the second portion.

15. The composition of claim 13, wherein the heterologous sequence comprises a DNA sequence, an RNA sequence, or a DNA/RNA hybrid sequence.

16. The composition of claim 13, wherein the heterologous sequence is an aptamer.

17. The composition of claim 1 or claim 2, wherein the direct repeat sequence comprises a stem-loop structure proximal to a 3′ end of the direct repeat sequence, wherein the stem-loop structure comprises a first stem nucleotide strand, a second stem nucleotide strand, and a loop nucleotide strand between the first stem nucleotide strand and the second stem nucleotide strand.

18. The composition of claim 17, wherein the first stem nucleotide strand comprises 3 to 5 nucleotides, the second stem nucleotide strand comprises 3 to 5 nucleotides, and the loop nucleotide strand comprises 7 to 11 nucleotides.

19. The composition of claim 17, wherein at least 3 nucleotides of the first stem nucleotide strand are complementary to at least 3 nucleotides of the second nucleotide stem strand.

20. The composition of claim 17, wherein at least 4 nucleotides of the first stem nucleotide strand are complementary to at least 4 nucleotides of the second nucleotide stem strand.

21. The composition of claim 17, wherein the first stem nucleotide strand is substantially complementary to the second nucleotide stem strand.

22. The composition of claim 1 or claim 2, wherein the Cas12i2 polypeptide comprises an amino acid sequence with at least 90% identity to SEQ ID NO: 2.

23. The composition of claim 1 or claim 2, wherein the Cas12i2 polypeptide comprises an amino acid sequence with at least 95% identity to SEQ ID NO: 2.

24. The composition of claim 1 or claim 2, wherein the Cas12i2 polypeptide comprises an amino acid sequence set forth in any one of SEQ ID NOs: 3-7.

25. The composition of claim 1 or claim 2, wherein the Cas12i2 polypeptide comprises at least one of an epitope peptide, a nuclear localization signal, and a nuclear export signal.

26. The composition of claim 1 or claim 2, wherein the target sequence is present in a cell.

27. A composition of claim 1 or claim 2, wherein the composition is formulated for delivery to a cell.

28. The composition of claim 1 or claim 2, wherein the Cas12i2 polypeptide and the RNA guide are encoded in a vector, e.g., one or more expression vectors.

29. The composition of claim 1 or claim 2, wherein the composition demonstrates increased binding to the target sequence adjacent to the PAM sequence comprising 4 nucleotides, as compared to a sequence that is not adjacent to a PAM sequence of the disclosure.

30. The composition of claim 1 or claim 2, wherein the composition demonstrates increased binding affinity to the target sequence, as compared to a sequence that is not adjacent to a PAM sequence of the disclosure.

31. The composition of claim 1 or claim 2, wherein the composition demonstrates increased RNA-DNA interactions with the target sequence, as compared to a sequence that is not adjacent to a PAM sequence of the disclosure.

32. The composition of claim 1 or claim 2, wherein the composition demonstrates decreased dissociation from the target sequence, as compared to a sequence that is not adjacent to a PAM sequence of the disclosure.

33. The composition of claim 1 or claim 2, wherein the composition demonstrates increased enzymatic activity at the target sequence, as compared to a sequence that is not adjacent to a PAM sequence of the disclosure.

34. The composition of claim 1 or claim 2, wherein the composition demonstrates decreased binding or binding affinity to a non-target sequence, as compared to a composition that binds a sequence that is not adjacent to a PAM sequence of the disclosure.

35. The composition of claim 29, wherein the PAM sequence of the disclosure does not comprise the sequence 5′-NVVN-3′, wherein V is any nucleotide except for T.

36. A vector comprising a sequence encoding the Cas12i2 polypeptide and RNA guide of the composition of claim 1 or claim 2.

37. A cell comprising the composition of claim 1 or claim 2.

38. A method of expressing the vector of claim 28.

39. A method of producing composition of claim 1 or claim 2.

40. A method of delivering the composition of claim 1 or claim 2.

41. A method of binding the composition of claim 1 or claim 2 with the target sequence.

42. A method of targeting a sequence adjacent to a PAM sequence comprising four nucleotides, the method comprising contacting the sequence with a composition of claim 1 or claim 2.

43. The method of claim 42, wherein the PAM sequence comprises the sequence 5′-NTTN-3′, wherein N is any nucleotide.

44. A method of designing an RNA guide for targeting a target sequence, the method comprising identifying a PAM sequence comprising the sequence 5′-NTTN-3′ adjacent to the target sequence, wherein N is any nucleotide, and designing or preparing a spacer sequence to be substantially complementary to the target sequence.

45. The method of claim 42, wherein the PAM sequence comprises the sequence 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′, wherein N is any nucleotide, Y is C or T, B is any nucleotide except for A, D is any nucleotide except for C, and R is A or G.

46. The method of claim 45, wherein the PAM sequence comprises the sequence 5′-CTTT-3′, 5′-CTTC-3′, 5′-GTTT-3′, 5′-GTTC-3′, 5′-TTTC-3′, 5′-GTTA-3′, or 5′-GTTG-3′.

47. A kit or system comprising a composition of claim 1, a cell comprising the composition, or a vector encoding the Cas12i2 polypeptide and RNA guide of the composition.


SEQUENCE LISTING (175 sequences)

SEQ ID NO: 1 - DNA, 3162 bp, Artificial Sequence
  Synthetic Construct
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 2 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 3 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 4 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 5 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 6 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 7 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 8 - RNA, 36 nt, Artificial Sequence
  Synthetic Construct
guugcaaaacccaagaaauccgucuuucauugacgg

SEQ ID NO: 9 - RNA, 36 nt, Artificial Sequence
  Synthetic Construct
aauagcggcccuaagaaauccgucuuucauugacgg

SEQ ID NO: 10 - RNA, 36 nt, Artificial Sequence
  Synthetic Construct
auuggaacuggcgagaaauccgucuuucauugacgg

SEQ ID NO: 11 - RNA, 36 nt, Artificial Sequence
  Synthetic Construct
ccagcaacaccuaagaaauccgucuuucauugacgg

SEQ ID NO: 12 - RNA, 36 nt, Artificial Sequence
  Synthetic Construct
cggcgcucgaauaggaaauccgucuuucauugacgg

SEQ ID NO: 13 - RNA, 36 nt, Artificial Sequence
  Synthetic Construct
guggcaacaccuaagaaauccgucuuucauugacgg

SEQ ID NO: 14 - RNA, 36 nt, Artificial Sequence
  Synthetic Construct
guugcaacaccuaagaaauccgucuuucauugacgg

SEQ ID NO: 15 - RNA, 36 nt, Artificial Sequence
  Synthetic Construct
guugcaaugccuaagaaauccgucuuucauugacgg

SEQ ID NO: 16 - RNA, 34 nt, Artificial Sequence
  Synthetic Construct
gcaacaccuaagaaauccgucuuucauugacggg

SEQ ID NO: 17 - RNA, 23 nt, Artificial Sequence
  Synthetic Construct
agaaauccgucuuucauugacgg

SEQ ID NO: 18 - RNA, 23 nt, Artificial Sequence
  Synthetic Construct
  misc_feature (1)..(1): n is a or g
ngaaauccgucuuucauugacgg

SEQ ID NO: 19 - RNA, 17 nt, Artificial Sequence
  Synthetic Construct
ccgucuuucauugacgg

SEQ ID NO: 20 - DNA, 36 bp, Artificial Sequence
  Synthetic Construct
gttgcaaaacccaagaaatccgtctttcattgacgg

SEQ ID NO: 21 - DNA, 15 bp, Artificial Sequence
  Synthetic Construct
ggaaaactccctttg

SEQ ID NO: 22 - DNA, 20 bp, Artificial Sequence
  Synthetic Construct
tgagaatggtgcgtcctagg

SEQ ID NO: 23 - DNA, 15 bp, Artificial Sequence
  Synthetic Construct
tgttcaccaggtcgt

SEQ ID NO: 24 - DNA, 15 bp, Artificial Sequence
  Synthetic Construct
cctcttccgatgttg

SEQ ID NO: 25 - DNA, 20 bp, Artificial Sequence
  Synthetic Construct
agcccctccagccggtcctg

SEQ ID NO: 26 - DNA, 15 bp, Artificial Sequence
  Synthetic Construct
gactttgtctccttc

SEQ ID NO: 27 - DNA, 15 bp, Artificial Sequence
  Synthetic Construct
tctgtctgcagcttg

SEQ ID NO: 28 - DNA, 20 bp, Artificial Sequence
  Synthetic Construct
tggcctgggtcacctctacg

SEQ ID NO: 29 - DNA, 15 bp, Artificial Sequence
  Synthetic Construct
gctggcccagatcct

SEQ ID NO: 30 - DNA, 15 bp, Artificial Sequence
  Synthetic Construct
ataccgtcggcgttg

SEQ ID NO: 31 - DNA, 20 bp, Artificial Sequence
  Synthetic Construct
gtggagtccagcacggcgcg

SEQ ID NO: 32 - DNA, 15 bp, Artificial Sequence
  Synthetic Construct
ggcgggcggcggcgc

SEQ ID NO: 33 - DNA, 15 bp, Artificial Sequence
  Synthetic Construct
gacggcatggggttg

SEQ ID NO: 34 - DNA, 20 bp, Artificial Sequence
  Synthetic Construct
ggtgagggaggagagatgcc

SEQ ID NO: 35 - DNA, 15 bp, Artificial Sequence
  Synthetic Construct
cggagaggacccaga

SEQ ID NO: 36 - DNA, 15 bp, Artificial Sequence
  Synthetic Construct
taggcagattcctta

SEQ ID NO: 37 - DNA, 20 bp, Artificial Sequence
  Synthetic Construct
tctggtgacacacccccatt

SEQ ID NO: 38 - DNA, 15 bp, Artificial Sequence
  Synthetic Construct
tcctggagccatctc

SEQ ID NO: 39 - DNA, 15 bp, Artificial Sequence
  Synthetic Construct
gccgaggcggccttc

SEQ ID NO: 40 - DNA, 20 bp, Artificial Sequence
  Synthetic Construct
gtgagtggcttccctgccgc

SEQ ID NO: 41 - DNA, 15 bp, Artificial Sequence
  Synthetic Construct
ggccgccgcgggcgc

SEQ ID NO: 42 - DNA, 15 bp, Artificial Sequence
  Synthetic Construct
gcatataccagtttg

SEQ ID NO: 43 - DNA, 20 bp, Artificial Sequence
  Synthetic Construct
tggataaaacttctcggagg

SEQ ID NO: 44 - DNA, 15 bp, Artificial Sequence
  Synthetic Construct
gttactcagatcagt

SEQ ID NO: 45 - DNA, 15 bp, Artificial Sequence
  Synthetic Construct
cctgactgttccttg

SEQ ID NO: 46 - DNA, 20 bp, Artificial Sequence
  Synthetic Construct
tgtgacctgttcccacatct

SEQ ID NO: 47 - DNA, 15 bp, Artificial Sequence
  Synthetic Construct
ggatgggctgcagga

SEQ ID NO: 48 - DNA, 15 bp, Artificial Sequence
  Synthetic Construct
ccaagccagagattg

SEQ ID NO: 49 - DNA, 20 bp, Artificial Sequence
  Synthetic Construct
tgtgagggcctagtggggtg

SEQ ID NO: 50 - DNA, 15 bp, Artificial Sequence
  Synthetic Construct
ttcattgagacaggc

SEQ ID NO: 51 - DNA, 15 bp, Artificial Sequence
  Synthetic Construct
tttcttaacgtattg

SEQ ID NO: 52 - DNA, 20 bp, Artificial Sequence
  Synthetic Construct
agaggtgggaatcaggccca

SEQ ID NO: 53 - DNA, 15 bp, Artificial Sequence
  Synthetic Construct
ggtagttcaatggga

SEQ ID NO: 54 - DNA, 15 bp, Artificial Sequence
  Synthetic Construct
acggctggcgtgttc

SEQ ID NO: 55 - DNA, 20 bp, Artificial Sequence
  Synthetic Construct
tcttgagatgggctcgggct

SEQ ID NO: 56 - DNA, 15 bp, Artificial Sequence
  Synthetic Construct
acttggccagcttca

SEQ ID NO: 57 - DNA, 15 bp, Artificial Sequence
  Synthetic Construct
gggggagggtggttg

SEQ ID NO: 58 - DNA, 20 bp, Artificial Sequence
  Synthetic Construct
gtgcccttcggtcctcggca

SEQ ID NO: 59 - DNA, 15 bp, Artificial Sequence
  Synthetic Construct
cccccctccgtctcc

SEQ ID NO: 60 - DNA, 15 bp, Artificial Sequence
  Synthetic Construct
gtggtgggcatattc

SEQ ID NO: 61 - DNA, 20 bp, Artificial Sequence
  Synthetic Construct
tgtgcccgtggggacccccg

SEQ ID NO: 62 - DNA, 15 bp, Artificial Sequence
  Synthetic Construct
gttgtgtcctgttcg

SEQ ID NO: 63 - DNA, 15 bp, Artificial Sequence
  Synthetic Construct
ggagcgtgtacgttg

SEQ ID NO: 64 - DNA, 20 bp, Artificial Sequence
  Synthetic Construct
gtgcccgctgctgtctaatg

SEQ ID NO: 65 - DNA, 15 bp, Artificial Sequence
  Synthetic Construct
ccctggagcctccct

SEQ ID NO: 66 - DNA, 15 bp, Artificial Sequence
  Synthetic Construct
aaagttcatggtttc

SEQ ID NO: 67 - DNA, 20 bp, Artificial Sequence
  Synthetic Construct
ggaggcccgaccggggccgg

SEQ ID NO: 68 - DNA, 15 bp, Artificial Sequence
  Synthetic Construct
cgcggctcgcgctcc

SEQ ID NO: 69 - DNA, 15 bp, Artificial Sequence
  Synthetic Construct
attttcagaggcttg

SEQ ID NO: 70 - DNA, 20 bp, Artificial Sequence
  Synthetic Construct
tgagtgctccgtgttaaggg

SEQ ID NO: 71 - DNA, 15 bp, Artificial Sequence
  Synthetic Construct
gcaggtaggatgggg

SEQ ID NO: 72 - DNA, 15 bp, Artificial Sequence
  Synthetic Construct
caaacaaatgctttc

SEQ ID NO: 73 - DNA, 20 bp, Artificial Sequence
  Synthetic Construct
tccgctctgagcaaggccca

SEQ ID NO: 74 - DNA, 15 bp, Artificial Sequence
  Synthetic Construct
cagggactgcaaaaa

SEQ ID NO: 75 - DNA, 8 bp, Artificial Sequence
  Synthetic Construct
  misc_feature (1)..(8): n is any nucleotide (A, T, C, or G)
nnnnnnnn

SEQ ID NO: 76 - DNA, 20 bp, Artificial Sequence
  Synthetic Construct
tgagaatggtgcgtcctagg

SEQ ID NO: 77 - DNA, 20 bp, Artificial Sequence
  Synthetic Construct
agcccctccagccggtcctg

SEQ ID NO: 78 - DNA, 20 bp, Artificial Sequence
  Synthetic Construct
tggcctgggtcacctctacg

SEQ ID NO: 79 - DNA, 20 bp, Artificial Sequence
  Synthetic Construct
gtggagtccagcacggcgcg

SEQ ID NO: 80 - DNA, 20 bp, Artificial Sequence
  Synthetic Construct
ggtgagggaggagagatgcc

SEQ ID NO: 81 - DNA, 20 bp, Artificial Sequence
  Synthetic Construct
tctggtgacacacccccatt

SEQ ID NO: 82 - DNA, 20 bp, Artificial Sequence
  Synthetic Construct
gtgagtggcttccctgccgc

SEQ ID NO: 83 - DNA, 20 bp, Artificial Sequence
  Synthetic Construct
tggataaaacttctcggagg

SEQ ID NO: 84 - DNA, 20 bp, Artificial Sequence
  Synthetic Construct
tgtgacctgttcccacatct

SEQ ID NO: 85 - DNA, 20 bp, Artificial Sequence
  Synthetic Construct
tgtgagggcctagtggggtg

SEQ ID NO: 86 - DNA, 20 bp, Artificial Sequence
  Synthetic Construct
agaggtgggaatcaggccca

SEQ ID NO: 87 - DNA, 20 bp, Artificial Sequence
  Synthetic Construct
tcttgagatgggctcgggct

SEQ ID NO: 88 - DNA, 20 bp, Artificial Sequence
  Synthetic Construct
gtgcccttcggtcctcggca

SEQ ID NO: 89 - DNA, 20 bp, Artificial Sequence
  Synthetic Construct
tgtgcccgtggggacccccg

SEQ ID NO: 90 - DNA, 20 bp, Artificial Sequence
  Synthetic Construct
gtgcccgctgctgtctaatg

SEQ ID NO: 91 - DNA, 20 bp, Artificial Sequence
  Synthetic Construct
ggaggcccgaccggggccgg

SEQ ID NO: 92 - DNA, 20 bp, Artificial Sequence
  Synthetic Construct
tgagtgctccgtgttaaggg

SEQ ID NO: 93 - DNA, 20 bp, Artificial Sequence
  Synthetic Construct
tccgctctgagcaaggccca

SEQ ID NO: 94 - DNA, 20 bp, Artificial Sequence
  Synthetic Construct
gtagcctctcccgctctggt

SEQ ID NO: 95 - DNA, 20 bp, Artificial Sequence
  Synthetic Construct
ggggaggcctggagtcatgg

SEQ ID NO: 96 - DNA, 43 bp, Artificial Sequence
  Synthetic Construct
agaaatccgtctttcattgacgggtagcctctcccgctctggt

SEQ ID NO: 97 - RNA, 43 nt, Artificial Sequence
  Synthetic Construct
agaaauccgucuuucauugacggcuagccucucccgcucuggu

SEQ ID NO: 98 - RNA, 43 nt, Artificial Sequence
  Synthetic Construct
agaaauccgucuuucauugacgggaagccucucccgcucuggu

SEQ ID NO: 99 - RNA, 43 nt, Artificial Sequence
  Synthetic Construct
agaaauccgucuuucauugacggguugccucucccgcucuggu

SEQ ID NO: 100 - RNA, 43 nt, Artificial Sequence
  Synthetic Construct
agaaauccgucuuucauugacggguacccucucccgcucuggu

SEQ ID NO: 101 - RNA, 43 nt, Artificial Sequence
  Synthetic Construct
agaaauccgucuuucauugacggguaggcucucccgcucuggu

SEQ ID NO: 102 - RNA, 43 nt, Artificial Sequence
  Synthetic Construct
agaaauccgucuuucauugacggguagcgucucccgcucuggu

SEQ ID NO: 103 - RNA, 43 nt, Artificial Sequence
  Synthetic Construct
agaaauccgucuuucauugacggguagccacucccgcucuggu

SEQ ID NO: 104 - RNA, 43 nt, Artificial Sequence
  Synthetic Construct
agaaauccgucuuucauugacggguagccugucccgcucuggu

SEQ ID NO: 105 - RNA, 43 nt, Artificial Sequence
  Synthetic Construct
agaaauccgucuuucauugacggguagccucacccgcucuggu

SEQ ID NO: 106 - RNA, 43 nt, Artificial Sequence
  Synthetic Construct
agaaauccgucuuucauugacggguagccucugccgcucuggu

SEQ ID NO: 107 - RNA, 43 nt, Artificial Sequence
  Synthetic Construct
agaaauccgucuuucauugacggguagccucucgcgcucuggu

SEQ ID NO: 108 - RNA, 43 nt, Artificial Sequence
  Synthetic Construct
agaaauccgucuuucauugacggguagccucuccggcucuggu

SEQ ID NO: 109 - RNA, 43 nt, Artificial Sequence
  Synthetic Construct
agaaauccgucuuucauugacggguagccucucccccucuggu

SEQ ID NO: 110 - RNA, 43 nt, Artificial Sequence
  Synthetic Construct
agaaauccgucuuucauugacggguagccucucccggucuggu

SEQ ID NO: 111 - RNA, 43 nt, Artificial Sequence
  Synthetic Construct
agaaauccgucuuucauugacggguagccucucccgcacuggu

SEQ ID NO: 112 - RNA, 43 nt, Artificial Sequence
  Synthetic Construct
agaaauccgucuuucauugacggguagccucucccgcuguggu

SEQ ID NO: 113 - RNA, 43 nt, Artificial Sequence
  Synthetic Construct
agaaauccgucuuucauugacggguagccucucccgcucaggu

SEQ ID NO: 114 - RNA, 43 nt, Artificial Sequence
  Synthetic Construct
agaaauccgucuuucauugacggguagccucucccgcucucgu

SEQ ID NO: 115 - RNA, 43 nt, Artificial Sequence
  Synthetic Construct
agaaauccgucuuucauugacggguagccucucccgcucugcu

SEQ ID NO: 116 - RNA, 43 nt, Artificial Sequence
  Synthetic Construct
agaaauccgucuuucauugacggguagccucucccgcucugga

SEQ ID NO: 117 - RNA, 43 nt, Artificial Sequence
  Synthetic Construct
agaaauccgucuuucauugacggcaagccucucccgcucuggu

SEQ ID NO: 118 - RNA, 43 nt, Artificial Sequence
  Synthetic Construct
agaaauccgucuuucauugacgggaugccucucccgcucuggu

SEQ ID NO: 119 - RNA, 43 nt, Artificial Sequence
  Synthetic Construct
agaaauccgucuuucauugacggguucccucucccgcucuggu

SEQ ID NO: 120 - RNA, 43 nt, Artificial Sequence
  Synthetic Construct
agaaauccgucuuucauugacggguacgcucucccgcucuggu

SEQ ID NO: 121 - RNA, 43 nt, Artificial Sequence
  Synthetic Construct
agaaauccgucuuucauugacggguagggucucccgcucuggu

SEQ ID NO: 122 - RNA, 43 nt, Artificial Sequence
  Synthetic Construct
agaaauccgucuuucauugacggguagcgacucccgcucuggu

SEQ ID NO: 123 - RNA, 43 nt, Artificial Sequence
  Synthetic Construct
agaaauccgucuuucauugacggguagccagucccgcucuggu

SEQ ID NO: 124 - RNA, 43 nt, Artificial Sequence
  Synthetic Construct
agaaauccgucuuucauugacggguagccugacccgcucuggu

SEQ ID NO: 125 - RNA, 43 nt, Artificial Sequence
  Synthetic Construct
agaaauccgucuuucauugacggguagccucagccgcucuggu

SEQ ID NO: 126 - RNA, 43 nt, Artificial Sequence
  Synthetic Construct
agaaauccgucuuucauugacggguagccucuggcgcucuggu

SEQ ID NO: 127 - RNA, 43 nt, Artificial Sequence
  Synthetic Construct
agaaauccgucuuucauugacggguagccucucgggcucuggu

SEQ ID NO: 128 - RNA, 43 nt, Artificial Sequence
  Synthetic Construct
agaaauccgucuuucauugacggguagccucuccgccucuggu

SEQ ID NO: 129 - RNA, 43 nt, Artificial Sequence
  Synthetic Construct
agaaauccgucuuucauugacggguagccucuccccgucuggu

SEQ ID NO: 130 - RNA, 43 nt, Artificial Sequence
  Synthetic Construct
agaaauccgucuuucauugacggguagccucucccggacuggu

SEQ ID NO: 131 - RNA, 43 nt, Artificial Sequence
  Synthetic Construct
agaaauccgucuuucauugacggguagccucucccgcaguggu

SEQ ID NO: 132 - RNA, 43 nt, Artificial Sequence
  Synthetic Construct
agaaauccgucuuucauugacggguagccucucccgcugaggu

SEQ ID NO: 133 - RNA, 43 nt, Artificial Sequence
  Synthetic Construct
agaaauccgucuuucauugacggguagccucucccgcucacgu

SEQ ID NO: 134 - RNA, 43 nt, Artificial Sequence
  Synthetic Construct
agaaauccgucuuucauugacggguagccucucccgcucuccu

SEQ ID NO: 135 - RNA, 43 nt, Artificial Sequence
  Synthetic Construct
agaaauccgucuuucauugacggguagccucucccgcucugca

SEQ ID NO: 136 - RNA, 43 nt, Artificial Sequence
  Synthetic Construct
agaaauccgucuuucauugacggggggaggccuggagucaugg

SEQ ID NO: 137 - RNA, 43 nt, Artificial Sequence
  Synthetic Construct
agaaauccgucuuucauugacggcgggaggccuggagucaugg

SEQ ID NO: 138 - RNA, 43 nt, Artificial Sequence
  Synthetic Construct
agaaauccgucuuucauugacgggcggaggccuggagucaugg

SEQ ID NO: 139 - RNA, 43 nt, Artificial Sequence
  Synthetic Construct
agaaauccgucuuucauugacggggcgaggccuggagucaugg

SEQ ID NO: 140 - RNA, 43 nt, Artificial Sequence
  Synthetic Construct
agaaauccgucuuucauugacgggggcaggccuggagucaugg

SEQ ID NO: 141 - RNA, 43 nt, Artificial Sequence
  Synthetic Construct
agaaauccgucuuucauugacgggggguggccuggagucaugg

SEQ ID NO: 142 - RNA, 43 nt, Artificial Sequence
  Synthetic Construct
agaaauccgucuuucauugacggggggacgccuggagucaugg

SEQ ID NO: 143 - RNA, 43 nt, Artificial Sequence
  Synthetic Construct
agaaauccgucuuucauugacggggggagcccuggagucaugg

SEQ ID NO: 144 - RNA, 43 nt, Artificial Sequence
  Synthetic Construct
agaaauccgucuuucauugacggggggagggcuggagucaugg

SEQ ID NO: 145 - RNA, 43 nt, Artificial Sequence
  Synthetic Construct
agaaauccgucuuucauugacggggggaggcguggagucaugg

SEQ ID NO: 146 - RNA, 43 nt, Artificial Sequence
  Synthetic Construct
agaaauccgucuuucauugacggggggaggccaggagucaugg

SEQ ID NO: 147 - RNA, 43 nt, Artificial Sequence
  Synthetic Construct
agaaauccgucuuucauugacggggggaggccucgagucaugg

SEQ ID NO: 148 - RNA, 43 nt, Artificial Sequence
  Synthetic Construct
agaaauccgucuuucauugacggggggaggccugcagucaugg

SEQ ID NO: 149 - RNA, 43 nt, Artificial Sequence
  Synthetic Construct
agaaauccgucuuucauugacggggggaggccuggugucaugg

SEQ ID NO: 150 - RNA, 43 nt, Artificial Sequence
  Synthetic Construct
agaaauccgucuuucauugacggggggaggccuggacucaugg

SEQ ID NO: 151 - RNA, 43 nt, Artificial Sequence
  Synthetic Construct
agaaauccgucuuucauugacggggggaggccuggagacaugg

SEQ ID NO: 152 - RNA, 43 nt, Artificial Sequence
  Synthetic Construct
agaaauccgucuuucauugacggggggaggccuggagugaugg

SEQ ID NO: 153 - RNA, 43 nt, Artificial Sequence
  Synthetic Construct
agaaauccgucuuucauugacggggggaggccuggagucuugg

SEQ ID NO: 154 - RNA, 43 nt, Artificial Sequence
  Synthetic Construct
agaaauccgucuuucauugacggggggaggccuggagucaagg

SEQ ID NO: 155 - RNA, 43 nt, Artificial Sequence
  Synthetic Construct
agaaauccgucuuucauugacggggggaggccuggagucaucg

SEQ ID NO: 156 - RNA, 43 nt, Artificial Sequence
  Synthetic Construct
agaaauccgucuuucauugacggggggaggccuggagucaugc

SEQ ID NO: 157 - RNA, 43 nt, Artificial Sequence
  Synthetic Construct
agaaauccgucuuucauugacggccggaggccuggagucaugg

SEQ ID NO: 158 - RNA, 43 nt, Artificial Sequence
  Synthetic Construct
agaaauccgucuuucauugacgggccgaggccuggagucaugg

SEQ ID NO: 159 - RNA, 43 nt, Artificial Sequence
  Synthetic Construct
agaaauccgucuuucauugacggggccaggccuggagucaugg

SEQ ID NO: 160 - RNA, 43 nt, Artificial Sequence
  Synthetic Construct
agaaauccgucuuucauugacgggggcuggccuggagucaugg

SEQ ID NO: 161 - RNA, 43 nt, Artificial Sequence
  Synthetic Construct
agaaauccgucuuucauugacggggggucgccuggagucaugg

SEQ ID NO: 162 - RNA, 43 nt, Artificial Sequence
  Synthetic Construct
agaaauccgucuuucauugacggggggaccccuggagucaugg

SEQ ID NO: 163 - RNA, 43 nt, Artificial Sequence
  Synthetic Construct
agaaauccgucuuucauugacggggggagcgcuggagucaugg

SEQ ID NO: 164 - RNA, 43 nt, Artificial Sequence
  Synthetic Construct
agaaauccgucuuucauugacggggggagggguggagucaugg

SEQ ID NO: 165 - RNA, 43 nt, Artificial Sequence
  Synthetic Construct
agaaauccgucuuucauugacggggggaggcgaggagucaugg

SEQ ID NO: 166 - RNA, 43 nt, Artificial Sequence
  Synthetic Construct
agaaauccgucuuucauugacggggggaggccacgagucaugg

SEQ ID NO: 167 - RNA, 43 nt, Artificial Sequence
  Synthetic Construct
agaaauccgucuuucauugacggggggaggccuccagucaugg

SEQ ID NO: 168 - RNA, 43 nt, Artificial Sequence
  Synthetic Construct
agaaauccgucuuucauugacggggggaggccugcugucaugg

SEQ ID NO: 169 - RNA, 43 nt, Artificial Sequence
  Synthetic Construct
agaaauccgucuuucauugacggggggaggccuggucucaugg

SEQ ID NO: 170 - RNA, 43 nt, Artificial Sequence
  Synthetic Construct
agaaauccgucuuucauugacggggggaggccuggacacaugg

SEQ ID NO: 171 - RNA, 43 nt, Artificial Sequence
  Synthetic Construct
agaaauccgucuuucauugacggggggaggccuggagagaugg

SEQ ID NO: 172 - RNA, 43 nt, Artificial Sequence
  Synthetic Construct
agaaauccgucuuucauugacggggggaggccuggaguguugg

SEQ ID NO: 173 - RNA, 43 nt, Artificial Sequence
  Synthetic Construct
agaaauccgucuuucauugacggggggaggccuggagucuagg

SEQ ID NO: 174 - RNA, 43 nt, Artificial Sequence
  Synthetic Construct
agaaauccgucuuucauugacggggggaggccuggagucaacg

SEQ ID NO: 175 - RNA, 43 nt, Artificial Sequence
  Synthetic Construct
agaaauccgucuuucauugacggggggaggccuggagucaucc